(19) United States
(12) Patent Application Publication
(10) Pub. No.: US 2022/0401475 A1
(43) Pub. Date: Dec. 22, 2022

(54) COMBINATIONS OF MULTIPLE CHIMERIC ANTIGEN RECEPTORS FOR IMMUNOTHERAPY

(71) Applicant: MEMORIAL SLOAN-KETTERING CANCER CENTER, New York, NY (US)

(72) Inventors: Michel Sadelain, New York, NY (US); Mohamad Hamieh, New York, NY (US); Judith Feucht, Tuebingen (DE)

(73) Assignee: MEMORIAL SLOAN-KETTERING CANCER CENTER, New York, NY (US)

(21) Appl. No.: 17/405,613

(22) Filed: Aug. 18, 2021

Related U.S. Application Data
(63) Continuation of application No. PCT/US2020/018662, filed on Feb. 18, 2020.
(60) Provisional application No. 62/807,181, filed on Feb. 18, 2019.

(51) Int. Cl. A61K 35/17; C07K 14/725; C07K 14/705; C07K 16/28

(52) U.S. Cl. CPC A61K 35/17; C07K 14/7051; A61K 38/00; C07K 14/70578; C07K 16/2803; C07K 14/70521

(57) ABSTRACT

The presently disclosed subject matter provides methods and compositions for enhancing the immune response toward tumor and pathogen antigens. It relates to immunoresponsive cells comprising two or more chimeric antigen receptors (CARs), wherein the CARs comprise different intracellular signaling domains, in particular, the intracellular signaling domains of the CARs comprise different co-stimulatory molecules.

### 1. CROSS-REFERENCE TO RELATED APPLICATIONS

This application is a Continuation of International Patent Application No. PCT/US20/18662, filed on Feb. 18, 2020, which claims priority to U.S. Provisional Patent Application Ser. No. 62/807,181, filed on Feb. 18, 2019, the contents of each of which are incorporated herein in their entireties, and to each of which priority is claimed.

### 2. SEQUENCE LISTING

The specification further incorporates by reference the Sequence Listing submitted herewith via EFS on Aug. 18, 2021. Pursuant to 37 C.F.R. § 1.52(e)(5), the Sequence Listing text file, identified as 0727341270SL.txt, is 172,861 bytes and was created on Aug. 18, 2021. The Sequence Listing, electronically filed herewith, does not extend beyond the scope of the specification and thus does not contain new matter

### 3. INTRODUCTION

The presently disclosed subject matter provides methods and compositions for enhancing immune responses toward tumor and pathogen antigens. It relates to immunoresponsive cells comprising two or more chimeric antigen receptors (CARs), wherein the CARs comprise different intracellular signaling domains, in particular, the intracellular signaling domains of the CARs comprise different co-stimulatory molecules or portions thereof. The immunoresponsive cells have improved therapeutic efficacy.

### 4. BACKGROUND OF THE INVENTION

Chimeric antigen receptors (CARs) are synthetic receptors for antigen that reprogram T cell specificity, function and persistence¹. Patient-derived CAR T cells have demonstrated remarkable efficacy against a range of B cell malignancies^{1,2,3}, and early trial results suggest activity in multiple myeloma⁴. Despite high complete response rates, relapses occur in a large fraction of patients, some of which are antigen-negative and others antigen-low^{1,2,3,4,5,6,7,8}. Therefore, there remain needs of new strategies of CAR T cell therapy with improved efficacy and avoid tumor antigen escape.

### 5. SUMMARY OF THE INVENTION

The presently disclosed subject matter provides immunoresponsive cells comprising two or more CARs disclosed herein. In certain embodiments, the immunoresponsive cell comprises: a) a first CAR comprising a first extracellular antigen-binding domain that binds to a first antigen and a first intracellular signaling domain comprising a first co-stimulatory molecule or a portion thereof, wherein the first co-stimulatory molecule is CD28; and b) a second CAR comprising a second extracellular antigen-binding domain that binds to a second antigen and a second intracellular signaling domain comprising a second co-stimulatory molecule or a portion thereof, wherein the second co-stimulatory molecule is different from the first co-stimulatory molecule. In certain embodiments, the second co-stimulatory molecule is selected from the group consisting of 4-1BB, ICOS, OX40, DAP-10, CD27, CD40/My88, NKGD2, and combinations thereof. In certain embodiments, the second co-stimulatory molecule is 4-1BB. In certain embodiments, the first antigen is different from the second antigen.

The first antigen can have a density level of more than about 10,000 molecules per cell, between about 5,000 molecules per cell and about 10,000 molecules per cell, or less than about 5,000 molecules per cell on the surface of the target cell. In certain embodiments, the second antigen has a density level of less than about 5,000 molecules per cell on the surface of the target cell.

The second antigen can have a density level of more than about 10,000 molecules per cell, between about 5,000 molecules per cell and about 10,000 molecules per cell, or less than about 5,000 molecules per cell on the surface of the target cell. In certain embodiments, the second antigen has a density level of less than about 5,000 molecules per cell on the surface of the target cell.

The first intracellular signaling domain can comprise a native CD3ζ polypeptide or a modified CD3ζ polypeptide. The second intracellular signaling domain can comprise a native CD3ζ polypeptide or a modified CD3ζ polypeptide.

In certain embodiments, the modified CD3ζ polypeptide comprises one or more ITAM variants comprising one or more loss-of-function mutations, wherein each of the one or more ITAM variants is independently selected from the group consisting of an ITAM1 variant, an ITAM2 variant, and an ITAM3 variant. In certain embodiments, the modified CD3ζ polypeptide comprises or consists essentially of or consists of a native ITAM1, an ITAM2 variant and an ITAM3 variant. In certain embodiments, the native ITAM1 has the amino acid sequence set forth in SEQ ID NO: 23. In certain embodiments, the ITAM2 variant has the amino acid sequence set forth in SEQ ID NO: 29. In certain embodiments, the ITAM3 variant has the amino acid sequence set forth in SEQ ID NO: 33.

In certain embodiments, the modified CD3ζ polypeptide lacks all or part of immunoreceptor tyrosine-based activation motifs (ITAMs), wherein the ITAMs are ITAM1, ITAM2, and ITAM3. In certain embodiments, the modified CD3ζ polypeptide lacks ITAM2 and ITAM3.

In certain embodiments, the modified CD3ζ polypeptide comprises or consists essentially of or consists of a native ITAM1, an ITAM2 variant, and an ITAM3 variant. In certain embodiments, the native ITAM1 has the amino acid sequence set forth in SEQ ID NO: 23. In certain embodiments, the ITAM2 variant has the amino acid sequence set forth in SEQ ID NO: 29. In certain embodiments, the ITAM3 variant has the amino acid sequence set forth in SEQ ID NO: 33. In certain embodiments, the modified CD3ζ polypeptide has the amino acid sequence set forth in SEQ ID NO: 135.

In certain embodiments, the modified CD3ζ polypeptide comprises a native ITAM1, a deletion of ITAM2 or a portion thereof, and a deletion of ITAM3 or a portion thereof. In certain embodiments, the modified CD3ζ polypeptide has an amino acid sequence set forth in SEQ ID NO: 137.

In certain embodiments, the first antigen has a density of between about 5,000 molecules per cell and about 10,000 per cell molecules per cell, or more than about 10,000 molecules per cell on the surface of the target cell, and the first intracellular signaling domain comprises a modified CD3 polypeptide. In certain embodiments, the modified CD3ζ polypeptide comprises or consists essentially of or consists of a native ITAM1, an ITAM2 variant, and an ITAM3 variant. In certain embodiments, the native ITAM1 has the amino acid sequence set forth in SEQ ID NO: 23, the ITAM2 variant has the amino acid sequence set forth in SEQ ID NO: 29, and the ITAM3 variant has the amino acid sequence set forth in SEQ ID NO: 33. In certain embodiments, the modified CD3t polypeptide has the amino acid sequence set forth in SEQ ID NO: 135.

In certain embodiments, the first antigen has a density of less than about 5,000 molecules per cell on the surface of the target cell, and the first intracellular signaling domain comprises a native CD3ζ polypeptide.

In certain embodiments, the second antigen has a density level of more than about 10,000 molecules per cell on the surface of the target cell, and the second intracellular signaling domain comprises a modified CD3ζ polypeptide. In certain embodiments, the modified CD3ζ polypeptide comprises or consists essentially of or consists of a native ITAM1, an ITAM2 variant, and an ITAM3 variant. In certain embodiments, the native ITAM1 has the amino acid sequence set forth in SEQ ID NO: 23, the ITAM2 variant has the amino acid sequence set forth in SEQ ID NO: 29, and the ITAM3 variant has the amino acid sequence set forth in SEQ ID NO: 33. In certain embodiments, the modified CD3ζ polypeptide has the amino acid sequence set forth in SEQ ID NO: 135.

In certain embodiments, the second antigen has a density level of more than about 10,000 molecules per cell on the surface of the target cell, and the second intracellular signaling domain comprises two copies of a modified CD3ζ polypeptide or a native CD3ζ polypeptide.

In certain embodiments, the second antigen has a density level of between about 5,000 molecules per cell and about 10,000 per cell molecules per cell, or less than about 5,000 molecules per cell on the surface of the target cell, and the second intracellular signaling domain comprises a native CD3ζ polypeptide.

In certain embodiments, the immunoresponsive cell further comprises a third CAR comprising a third extracellular antigen-binding domain that binds to a third antigen and a third intracellular signaling domain. In certain embodiments, the third intracellular signaling domain does not comprise a costimulatory molecule. In certain embodiments, the third intracellular signaling domain comprises a costimulatory molecule or a portion thereof. In certain embodiments, the third costimulatory molecule comprises a native CD3ζ polypeptide or a modified CD3ζ polypeptide. The third antigen can have a density level of more than about 10,000 molecules per cell, between about 5,000 molecules per cell and about 10,000 molecules per cell, or less than about 5,000 molecules per cell on the surface of the target cell. The third costimulatory molecule can be selected from the group consisting of CD28, 4-1BB, ICOS, OX40, DAP-10, CD27, CD40/My88, NKGD2, and combinations thereof. In certain embodiments, the third costimulatory molecule comprises a CD28 polypeptide.

In certain embodiments, each of the first and second antigens is independently selected from tumor antigens, pathogen antigens, and combinations thereof In certain embodiments, each of the first and second antigens is a tumor antigen. In certain embodiments, the tumor antigen is selected from the group consisting of carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Eerb-B2, Erb-B3, Erb-B4, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), x-light chain, kinase insert domain receptor (KDR), Lewis Y (LeY), Ll cell adhesion molecule (L1CAM), melanoma antigen family A, 1 (MAGE-A1), Mucin 16 (MUC16), Mucin 1 (MUC1), Mesothelin (MSLN), MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, CCR4, CD5, CD3, TRBC1, TRBC2, TIM-3, Integrin B7, ICAM-1, and CLEC12A.

In certain embodiments, the first antigen is CD19 and the second antigen is CD22.

The presently disclosed subject matter provides compositions comprising an immunoresponsive cell disclosed herein. In certain embodiments, the composition is a pharmaceutical composition comprising a pharmaceutically acceptable excipient.

The presently disclosed immunoresponsive cells and pharmaceutical compositions comprising thereof can be used in a therapy, including, but not limited to, reducing tumor burden, treating and/or preventing a neoplasm, treating a subject having a relapse of a neoplasm, treating and/or preventing a pathogen infection, and/or treating and/or preventing an infectious disease.

The presently disclosed subject matter provides methods of reducing tumor burden in a subject. In certain embodiments, the method comprises administering to the subject an effective amount of the presently disclosed immunoresponsive cells or a pharmaceutical composition comprising thereof In certain embodiments, the method reduces the number of tumor cells, reduces tumor size, and/or eradicates the tumor in the subject.

The presently disclosed subject matter provides methods of treating and/or preventing a neoplasm. In certain embodiments, the method comprises administering to the subject an effective amount of the presently disclosed immunoresponsive cells or a pharmaceutical composition comprising thereof.

The presently disclosed subject matter also provides methods of treating a subject having a relapse of a neoplasm. In certain embodiments, the method comprises administering to the subject an effective amount of the presently disclosed immunoresponsive cells or the pharmaceutical composition comprising thereof. In certain embodiments, the subject received an immunotherapy prior to said administration. In certain embodiments, the immunotherapy comprises administration of immunoresponsive cells comprising a chimeric antigen receptor (CAR). In certain embodiments, the CAR comprises an intracellular signaling domain that comprises a co-stimulatory signaling region comprising a 4-1BB polypeptide.

In certain embodiments, the neoplasm or tumor is selected from the group consisting of blood cancer, B cell leukemia, multiple myeloma, Acute Myeloid Leukemia (AML), acute lymphoblastic leukemia (ALL), chronic lymphocytic leukemia, non-Hodgkin's lymphoma, and ovarian cancer. In certain embodiments, the neoplasm or tumor is B cell leukemia, multiple myeloma, Acute Myeloid Leukemia (AML), acute lymphoblastic leukemia (ALL), chronic lymphocytic leukemia, or non-Hodgkin's lymphoma, and the first antigen is CD19. In certain embodiments, the neoplasm or tumor is B cell leukemia, multiple myeloma, Acute Myeloid Leukemia (AML), acute lymphoblastic leukemia (ALL), chronic lymphocytic leukemia, or non-Hodgkin's lymphoma, and the first antigen is BCMA or ADGRE2. In certain embodiments, the neoplasm or tumor is a solid tumor. In certain embodiments, the first antigen is mesothelin (MSLN) or PSMA.

The presently disclosed subject matter provides nucleotide acid compositions. In certain embodiments, the nucleotide acid composition comprises a) a first nucleotide sequence encoding a first CAR comprising a first extracellular antigen-binding domain that binds to a first antigen and a first intracellular signaling domain comprising a first costimulatory molecule or a portion thereof, wherein the first co-stimulatory molecule is CD28; and b) a second nucleotide sequence encoding a second CAR comprising a second extracellular antigen-binding domain that binds to a second antigen and a second intracellular signaling domain comprising a second costimulatory molecule or a portion thereof, wherein the second co-stimulatory molecule is different from the first costimulatory molecule. In certain embodiments, the second co-stimulatory molecule is selected from the group consisting of 4-1BB, ICOS, OX40, DAP-10, CD27, CD40/My88, NKGD2, and combinations thereof. In certain embodiments, the second co-stimulatory molecule is 4-1BB. In certain embodiments, the first antigen is different from the second antigen. In certain embodiments, the nucleic acid composition is a vector. In certain embodiments, the nucleic acid composition is a DNA plasmid or a RNA fragment. In certain embodiments, the vector is selected from the group consisting of a retroviral vector, a lentiviral vector, an Adeno-associated viruses (AAV) vector, a non-viral vector, and combinations thereof. In certain embodiments, the vector is a retroviral vector. In certain embodiments, the non-viral vector is a transposon vector.

The presently disclosed subject matter provides methods of treating and/or preventing a pathogen infection in a subject. In certain embodiments, the method comprises administering to the subject an effective amount of the presently disclosed immunoresponsive cells or a pharmaceutical composition comprising thereof.

The presently disclosed subject matter provides methods of treating and/or preventing an infectious disease in a subject. In certain embodiments, the method comprises administering to the subject an effective amount of the presently disclosed immunoresponsive cells or a pharmaceutical composition comprising thereof.

In certain embodiments, the subject is an immunocompromised subject.

The presently disclosed subject matter provides methods for producing a presently disclosed immunoresponsive cell. In certain embodiments, the method comprises introducing into an immunoresponsive cell a presently disclosed nucleic acid composition.

The presently disclosed subject matter further provides kits comprising a presently disclosed immunoresponsive cell, a presently disclosed pharmaceutical composition, and/or a presently disclosed nucleic acid composition. In certain embodiments, the kit further comprises written instructions for treating and/or preventing a neoplasm, a pathogen infection, an infectious disease, an autoimmune disorder, and/or an allogeneic transplant.

### 6. BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A-1J: Trogocytic antigen extraction promotes tumor escape. FIG. 1A shows NALM6^{wt} leukaemia after CD19 CAR therapy (n=6-7 mice/group; two independent experiments are pooled for the 0.2×10⁶ CAR T cell dose). FIG. 1B shows CAR T cell counts, n=3-7 mice/group (left); EOMES/T-bet ratio (middle); PD-1, LAG-3 and TIM-3 expression in CAR T cells, n=3-5 mice/group days 32-70 (right). FIGS. 1C and 1D show CD19 surface quantification in NALM6′ c-left: day 14; n=3-7 mice/group; c-right: days 32-70; n=3-9 mice/group; d: ex-vivo cultured NALM6 retrieved from 19-BK treated mice treated (DO =time of retrieval; D6 =6 days after ex-vivo culture (n=9 independent samples); NALM6 cultured in vitro (n=4 independent samples). In b-d, cells harvested from bone marrow or extramedullary tumour sites of mice treated with 0.2×10⁶ CAR T cells. FIG. 1E shows CD19 MFI in NALM6-CD19-mCherry (middle) and CAR T cells (right; representative of 3 donors, n=3 independent samples). FIG. 1F shows heat map of protein expression in 19-28ζ-Trog^{Pos}, Trog^{Neg} and NALM6-CD19-mCherry cells. FIG. 1G shows CD19 MFI on blasts (left) and CAR+ T cells (right; n=4 patients samples). FIG. 1H shows percentage CAR T cells stained with cell tracer dye stained positive for IFNγ and Granzyme B (GzmB, n=4 donors). FIG. 1I shows ⁵¹Cr cytotoxic release assay (representative of 6 donors, n=3 independent samples). FIG. 1J shows percentage CAR T cells co-expressing PD-1, LAG-3 and TIM-3 (n=4 donors). NA=not available, i.e., no detectable cells. NT=non-treated mice. c, g, h and j, two-sided Mann-Whitney test; d, two-sided Wilcoxon matched-pairs signed rank test. All data represented as mean±SEM.

FIGS. 2A and 2B: Antigen density reduction differentially impacts CAR T cell activity via Kaplan-Meier survival analysis. FIG. 2A shows mice bearing NALM6^{wt} treated with 0.2×10⁶ 19-BBζ cells and infused 10 days later with either 0.5×10⁶ 19-28ζ or 19-BBζ cells (data representative of 3 independent experiments); FIG. 2B shows mice bearing NALM6-wt, -med or -low treated with 0.2×10⁶ CAR T cells (data pooled from two independent experiments). FIGS. 2A and 2B represent log-rank Mantel-Cox test.

FIGS. 3A-3I: CAR T cell cooperativity augments insufficient clonal tumour-lytic potential. FIG. 3A shows single-cell time-lapse imaging cytotoxicity assay. Time from CAR T-target cell conjugate formation (start point, T₀) to target cell death (determined by PI dye positivity, T_{PI}) recorded over 24 hours in wells containing 1 CAR T cell and 1 tumour cell (1:1 E:T ratio). FIG. 3B shows percentage wells with target killing (left). Time of conjugate formation to PI influx in target cells (lytic conjugates, right). FIG. 3C shows percentage non-lytic conjugate formation (Left); duration of non-lytic conjugates (Right). FIG. 3D shows cytotoxic time-lapse imaging with 2 CAR T cells and 1 tumour cell (2:1 E:T). Time from first conjugate formation (start point, T₀₋₁) to target cell death (T_{PI}) is indicated as ΔT1. Time from second conjugate formation (start point, T₀₋₂) to target cell death (T_{PI}) is indicated as ΔT2. FIG. 3E shows percentage wells with target killing in wells at 2:1 E:T ratio. FIG. 3F shows ratio of observed killing (P-obs_{2/1}) over estimated killing (P-est_{2/1}) in wells at 2:1 E:T ratio. FIG. 3G shows percentage target killing due to engagement of 2 CAR T cells with 1 tumour cell. FIG. 3H shows Time of lytic conjugate ΔT1 and ΔT2. In FIGS. 3B, 3C, 3E, 3F, 3G and 3H, n=5 donors. In FIGS. 3B, 3C and 3H, time is indicated in minutes. FIG. 31 shows percentage target killing in wells at 1:1 (left) and 2:1 E:T ratios (right), n=3 donors. In B-C, n≥197, e-h n≥242, (I) n≥70 (ratio 1:1) and n≥42 (ratio 2:1) individual wells were examined. In FIG. 3B-left, FIG. 3C-left, FIGS. 3 E, G and I, two-sided two-sample test of proportions. FIGS. 3B-right, 3C-right and 3H represents two-sided unpaired t-test. FIG. 3F represents two-sided Mann-Whitney test. All data represented as mean±SEM.

FIGS. 4A-4E: Rational combinatorial targeting overcomes antigen-low tumor escape. FIGS. 4A-4D depict Kaplan-Meier survival analysis. FIG. 4A shows mice bearing NALM6^{wt} treated with 0.2×10⁶ 19-BBζ cells followed by infusion of 0.5×10⁶ (left) or 1×10⁶ (right) CD22 CAR T cells 10 days later. FIGS. 4B-4D show mice bearing NALM6^{wt} (FIG. 4B) NALM6^{med} (FIG. 4C) and NALM6^{low} (FIG. 4D) treated with 0.2×10⁶ single or dual-targeted CAR T cells. FIG. 4E shows summary of combinatorial targeting strategies' outcomes based on relative antigens densities and CAR co-stimulatory design (based on median survival of treated mice in FIGS. 4B, 4C and 4D). FIGS. 4A-4D represent log-rank Mantel-Cox test.

FIGS. 5A-5H: In vitro and in vivo characterisation of 19-28ζ and 19-BBζ CAR T cells. FIG. 5A shows schematic representation of the CD19 CARs (scFv: single chain variable region specific for anti-CD19; EC: extracellular domain; TM: trans-membrane domain). FIGS. 5B and 5C Left: percentage of CAR+ T cells (left) and MFI (right) of T cell surface CAR expression. FIG. 5B shows CAR expression analysis performed 4 days after T cell transduction (n=9 donors). FIG. 5C shows CAR expression analysis performed 7 days after stimulation on NIH/3T3 cells expressing CD19 (n=10 donors). FIG. 5D shows 18 hour cytotoxic assay utilizing NALM6 target cells at multiple E:T ratios (n =3 donors). FIG. 5E shows Kaplan-Meier analysis of survival of NALM6-bearing mice treated with 1×10⁶, 0. 4×10⁶ or 0.2×10⁶ transduced CAR T cells. Corresponding BLI data are presented in FIG. 1A. FIG. 5F shows representative FACS profile of NALM6 and CAR T cells. NALM6 are GFP+. CAR T cells are LNGFR+. Absolute counts of NALM6 (left) and CAR T cells at day 14 post infusion (right; n=4 mice/group). FIG. 5G Left shows EOMES/T-bet ratio of CAR T cells. FIG. 5G Right shows fraction of CAR T cells expressing PD-1, LAG-3 and TIM-3 at day 14 post T cell infusion (n=4 mice/group). FIGS. 5F and 5G show that cells were isolated from mice bone marrow of mice treated with 0.2×10⁶ CAR T cells. FIG. 5H shows representative FACS profile of cells harvested by the time of relapse, 39 days after T cell infusion. (19-28ζ representative of 3 mice; 19-BBζ representative of 9 mice). NA=Not available (no detectable cells). FIG. 5E shows that 6 to 7 are mice/group pooled from two independent experiments for 0.2×10⁶ CAR T cell dose. FIGS. 5B, 5C, 5F and 5G represent two-sided Mann-Whitney test. FIG. 5E represents log-rank Mantel-Cox test. All data represented as mean±SEM.

FIGS. 6A-6G: Leukemic response and relapse patterns associated with SJ25C1 and FMC63 scFv-based CARs. FIG. 6A shows schematic representation of the CD19 CARs used using the scFv SJ25C1 or FMC63. (scFv: single chain variable fraction specific for CD19; EC: extracellular domain; TM: trans-membrane domain). FIGS. 6B and 6C show Kaplan-Meier analysis of survival of NALM6-bearing mice treated with 0.2×10⁶ transduced CART cells. FIG. 6D shows representative FACS profile of cells isolated by the time of relapse (data is representative of 3 mice/group). FIGS. 6E and 6F show representative FACS profile of surface CD19 expression on NALM6 cells isolated by the time of relapse (data is representative of 3 mice/group). FIG. 6E shows mice treated with SJ25C1-28ζ or FMC63-28ζ. FIG. 6F shows mce treated with SJ25C1-BBζ or FMC63-BBζ. FIG. 6G shows representative FACS profile of PD-1, LAG-3 and TIM-3 surface expression on BBζ CAR T cells isolated from relapsed mice (data is representative of 3 mice/group). For FIGS. 6B and 6C, 5 to 7 mice/group pooled from two independent experiments, and represent log-rank Mantel-Cox test. (One of the groups shown in FIG. 2B is one of 2 experiments merged in FIG. 1A.

FIGS. 7A-7I: CD19 trogocytosis in vitro and in vivo. FIG. 7A Left shows representative FACS profile of CD19 stained on CART cells isolated by the time of relapse from bone marrow or extramedullary tumor sites of mice treated with 19-BBζ CART cells. Right, MFI of CD19 staining gated on CAR T cells (19-BBζ, n=9 mice, control, n=4 mice). FIG. 7B shows CD19 mRNA expression in NALM6 of relapsed mice treated with 19-BBζ CART cells (Fold change DO vs NALM6 in vitro=0 .97; Padj=0.919; fold change D6 vs DO=0.625, Padj.=0.23; n=7 independent samples). FIG. 7C shows MFI of CD19 in the segregated NALM6 in a trans-well assay. NALM6 alone were plated at the top of the plate and NALM6 cells with CAR T cells at the bottom. Data are collected at days 1, 4, 7 and 14 (n=3 independent samples). FIG. 7D Left shows diagram illustrating co-culture of NALM6 cells and CAR T cell. Percentage and MFI of CD19 on NALM6 cell surface (middle) and on CAR T cell surface (right). Data were collected at 0, 1, 2 and 4 hours after co-culture gated on live singlet cells (n=4 donors). FIG. 7E shows MFI of CD19 on NALM6 cell surface after 2 hours of co-culture with 19-28ζ (left) or 19-BBζ (right) CAR T cells treated with F-Actin inhibitor “Latrunculin A” (n=5 donors). FIG. 7F shows intensity of heavy amino acid CD19-derived peptides detected in the lysate of FACS sorted cells after 1h of co-culture with NALM6-CD19-mcherry. NALM6-CD19-mCherry cells are used as a control (n=2 donors). FIG. 7G shows MFI of CD81 on NALM6 cell surface co-cultured with anti CD19 CART cells (n=3 donors). FIG. 7H shows MFI of CD22 on NALM6 cell surface co-cultured with anti-CD19 CAR T cells (data is representative of 3 donors, n=3 independent samples). FIG. 71 shows MFI of CD22 on cell surface of residual NALM6 retrieved from mice bone marrow 14 days after 19-BBζ CAR T cell treatment (0.2×10⁶ CAR T cell dose, n=3 mice). ns=non significant. FIGS. 7A, 7E and 7I represent two-sided Mann-Whitney test. FIG. 7B represents Binomial test, FDR-adjusted P values (Padj.). FIG. 7D represents 2-way ANOVA. Data are represented as mean±SEM.

FIGS. 8A-8E: Trogocytosis occurs with multiple targets and cell types. FIG. 8A Upper panel shows MFI of CD19 on cell surface of NALM6, SUB-P15, Raji and SK-OV-3-CD19⁺ cells co-cultured with 19-BBζ CART cells. FIG. 8A Lower panel shows MFI of CD19 on CAR T cell surface. (n=3 donors). FIG. 8B shows CD19 MFI on NALM6 co-cultured with ALL and CLL patients 19-28ζ CART cells. (n=4 patients samples). FIG. 8C shows MFI of CD22 on NALM6 cell surface (upper panel) and CD22-CAR T cells (lower panel). BCMA CAR T cells are used as control. FIG. 8D shows MFI of BCMA on KMS-12-BM cell surface (upper panel) and BCMA-CAR T cells (lower panel). CD22 CAR T cells are used as a control. FIG. 8E shows MFI of MSLN on A549 cell surface (upper panel) and MSLN-CAR T cells (lower panel). CD19-CAR T cells are used as a control. For FIGS. 8C, 8D and 8E, n=3 independent samples, data are representative of 3 donors. Data are represented as mean±SEM.

FIGS. 9A-9E: Therapeutic response and relapse patterns after CD22 CAR T cell treatment. FIG. 9A shows tumour burden was monitored using bioluminescence imaging (Average radiance [p/s/cm²/sr]) in mice bearing NALM6-GFP luciferase treated with 0.2×10⁶ CAR T cells 4 days later (n=7 mice/group). FIG. 9B shows absolute count of CAR T cells. FIG. 9C shows quantification of CD22 molecules on NALM6 cell surface. FIG. 9D shows expression of PD-1, LAG-3 and TIM-3 on CAR T cell surface. For FIGS. 9B, 9C, and 9D, cells were harvested from mice bone marrow at day 21 (relapse time, n=5 mice/group). FIG. 9E shows quantification of CD22 molecules on NALM6 surface 6 days after ex-vivo culture (CD22-28ζ and CD22-BBζ, n=5 independent samples/group, NALM6, n=3 independent samples). NA=Not available (no detectable cells). For FIGS. 9A, 9B, 9C and 9E, two-sided Mann-Whitney test. Data are represented as mean±SEM.

FIGS. 10A-10E: Functional effects of CD19 acquisition by CAR T cells. FIG. 10A shows diagram illustrating transduction of T cells with retroviral vector encoding CD19 under a PGK-100 promoter. Representative flow cytometry profile of CD19 expression on transduced T cells (n=6 donors). FIG. 10B shows percentage PD-1, LAG-3 and TIM-3 expression in Trog^{Pos} and Trog^{Neg} fractions (n=4 donors). FIG. 10C shows diagram illustrating co-expression of CD19 and CAR on T cells. T cells were transduced with SFG vector expressing CD19 followed by CAR transduction 24h later. After transduction, cells were left to in culture for 6 days. FIG. 10D shows absolute count of CAR T cells n=6 donors). FIG. 10E shows percentage of T cells co expression PD-1, LAG-3 and TIM-3 (n=6 donors). FIGS. 10B, 10D and 10E represent two-sided Mann-Whitney test. Data are represented as mean±SEM.

FIGS. 11A-11E: Internalized CD19/CAR complex after trogocytosis. FIG. 11A Left panel shows MFI of mCherry gated on CAR T cells after co-culture with NALM-6-CD19-mCherry. FIG. 11A Right panel shows MFI of CAR gated on CAR T cells (n=3 independent samples, data are representative of 3 donors). FIG. 11B Left shows representative FACS profile of CAR T cells stained with GAM and LNGFR in presence or absence of NALM6 cells (data is representative of 4 mice). FIG. 11B Right shows MFI of GAM on CAR T cells isolated from mice bone marrow or extramedullary tumour sites by the time of relapse (n=4 mice/group). FIG. 11C shows MFI of GAM on CAR T cells co-cultured with autologous blasts derived from refractory/relapsed ALL and CLL patients (n=4 patients samples). FIG. 11D shows representative confocal microscopy images of NALM6/CAR T cell conjugates. Data are representative of 3 donors (10 cells/experiments). FIG. 11E shows diagram illustrating CD19 trogocytosis by CAR T cells. FIGS. 11B and 11C represent two-sided Mann-Whitney test. Data are represented as mean±SEM.

FIGS. 12A-12D: Generation of NALM6 cell lines with graded CD19 expression. FIG. 12A shows genotype of edited CD19 locus in NALM6^{med} and NALM6^{low}. NALM6^{med} were obtained by monoallelic disruption of the CD19 gene using CRISPR/Cas9 system. Genotype of edited NALM6^{low}. NALM6-low were obtained by bi-allelic disruption of the CD19 gene. One of the allele encodes a stop codon (*), the second allele codes for a variant CD19 containing 3aa substitution (Mut-3aa). FIG. 12B shows prediction of the cleavage of CD19 protein sequence of the functional allele (Mut-3aa) in NALM6-low (left) and wt protein sequence (right) using signal 4.1 server. CD19gRNA were designed to target CD19 exon 1, next to the site of cleavage of signal peptide. Data were obtained by deep sequencing. FIG. 12C shows MFI of CD19 molecules on surface of NALM6-wt, -med and -low (n=3 independent experiment). FIG. 12D shows MFI of CD22 molecules on surface of NALM6-wt, -med and -low (n=3 independent experiment). For FIGS. 12C and 12D, data are represented as mean±SEM.

FIGS. 13A-13D: T cell cooperativity and antigen density determine CAR T cell killing capacity. Tumour lysis frequency in wells containing one CAR T cell:one tumour cell (R1:1) and one CAR T cell:two tumour cells (R2:1). FIG. 13A represents NALM6^{wt}, FIG. 13C represents NALM6^{med} and FIG. 13D represents NALM6^{low}. FIG. 13B shows estimated and observed killing percentage in wells containing one CAR T cell:two NALM6^{wt} (R2:1). Estimated percentage of killing is calculated as described in Materials and Methods. FIGS. 13A, 13C and 13D represent paired t test, and two-sided two-sample test of proportions. For FIG. 13A, n=5 donors. For FIGS. 13C and 13D, n=3 donors. FIGS. 13B represents one sided two-sample test of proportions (p<0.1 is used as significance level, n=5 donors). Data are represented as mean±SEM. Primary data in this figure are presented in FIG. 3.

FIGS. 14A-14H: Modelling of anticipated outcomes after single or combinatorial CAR T cell treatment schemas. Antigen-positive relapse (FIGS. 14A-14D) vs tumour control scenarios (FIGS. 14E-14H) upon single or combinatorial CAR T cell targeting. FIG. 14A shows antigen high relapse of residual tumour cells in the absence of residual CAR T cells. FIG. 14B shows antigen-low relapse in the presence of insensitive or exhausted CAR T cells. FIG. 14C shows tumor relapse after sequential combinatorial targeting failing against tumor cells with low antigen densities. Tumor rejection could overcome the A-C relapse scenarios by achieving a higher effector:target ratio (FIGS. 14E-14G), following higher T cell dose infusion or greater post-infusion expansion, operating through additive or cooperative tumor elimination. In FIGS. 14D and 14H, combinatorial targeting is mediated by dual-targeted CAR T cells, which may still fail in the face of low antigen densities (see FIG. 14D) depending on the costimulatory combinations (see FIG. 14H). In all cases, low antigen density may either be constitutively low or actively lowered following CAR T cell-mediated trogocytosis. Antigen-negative escapes are not represented.

FIG. 15 depicts non-limiting examples of combinations of the first CAR and the second CAR in accordance with certain embodiments of the presently disclosed subject matter.

### 7. DETAILED DESCRIPTION OF THE INVENTION

The presently disclosed subject matter provides methods and compositions for enhancing immune responses toward tumor and pathogen antigens. It relates to immunoresponsive cells comprising two or more CARs, wherein the CARs comprise different intracellular signaling domains, in particular, the intracellular signaling domains of the CARs comprise different costimulatory molecules or portions thereof. The presently disclosed immunoresponsive cells have improved therapeutic efficacy. The presently disclosed subject matter also provides methods of using such cells for inducing and/or enhancing an immune response to a target antigen (a tumor antigen or a pathogen antigen), and/or treating and/or preventing a neoplasm or other diseases/disorders where an increase in an antigen-specific immune response is desired. The presently disclosed subject matter is based, at least in part, on the discovery that immunoresponsive cells comprising two or more CARs, wherein the CARs comprise different intracellular signaling domains, in particular, the intracellular signaling domains of the CARs comprise different costimulatory molecules or portions thereof (e.g., one CAR's intracellular signaling domain comprises a CD28 polypeptide, and the other CAR's intracellular signaling domain comprises a 4-1BB polypeptide) exhibited unexpected synergistic effects (e.g., improved therapeutic potency (e.g., decreased cell exhaustion)) compared to cells comprising two or more CARs comprising the same intracellular signaling domains, e.g., optimized pairing of the co-stimulatory domains of the CARs.

Non-limiting embodiments of the presently disclosed subject matter are described by the present specification and Examples.

For purposes of clarity of disclosure and not by way of limitation, the detailed description is divided into the following subsections:
7.1. Definitions;
7.2. Chimeric Antigen Receptors;
7.3. Immunoresponsive Cells;
7.4. Nucleic Acid Compositions;
7.5. Genome Editing Methods
7.6 Polypeptides and Analogs;
7.7. Administration;
7.8. Formulations;
7.9. Methods of Treatment; and
7.10. Kits.

### 7.1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

By “immunoresponsive cell” is meant a cell that functions in an immune response or a progenitor, or progeny thereof

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when a receptor (e.g., a CAR) binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, 4-1BB (CD137), OX40, CD40, CD27, CD40/My88, NKGD2, ICOS, CD2, CD7, LIGHT, NKG2C, B7-H3, FcεRIγ, TNF receptor proteins, Immunoglobulin-like proteins, cytokine receptors, integrins, signaling lymphocytic activation molecules (SLAM proteins), activating NK cell receptors, BTLA, a Toll ligand receptor, CD30, CDS, ICAM-1, LFA-1 (CD11a/CD18), CDS, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11 a, LFA-1, ITGAM, CD11b, ITGAX, CD11 c, ITGB1, CD29, ITGB2, CD18, LFA-1, ITGB7, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immunoresponsive cell (e.g., a T-cell) in response to its binding to an antigen. Non-limiting examples of antigen-recognizing receptors include native or endogenous T cell receptors (“TCRs”), and chimeric antigen receptors (“CARs”).

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fc fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}:: V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imuno12009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chem 2003 25278(38):36740-7; Xie et al., Nat Biotech 1997 15(8):768-71; Ledbetter et al., Crit Rev Immunol1997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating and/or stimulating an immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “nucleic acid molecules” include any nucleic acid molecule that encodes a polypeptide of interest or a fragment thereof. Such nucleic acid molecules need not be 100% homologous or identical with an endogenous nucleic acid sequence, but may exhibit substantial identity. Polynucleotides having “substantial identity” or “substantial homology” to an endogenous sequence are typically capable of hybridizing with at least one strand of a double-stranded nucleic acid molecule. By “hybridize” is meant a pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, e.g., less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, e.g., at least about 50% formamide. Stringent temperature conditions will ordinarily include temperatures of at least about 30° C., at least about 37° C., or at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In certain embodiments, hybridization will occur at 30° C. in 750 mM NaCl, 75 mM trisodium citrate, and 1% SDS. In certain embodiments, hybridization will occur at 37° C. in 500 mM NaCl, 50 mM trisodium citrate, 1% SDS, 35% formamide, and 100 μg/ml denatured salmon sperm DNA (ssDNA). In certain embodiments, hybridization will occur at 42° C. in 250 mM NaCl, 25 mM trisodium citrate, 1% SDS, 50% formamide, and 200 pg/m1 ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps can be less than about 30 mM NaCl and 3 mM trisodium citrate, e.g., less than about 15 mM NaCl and 1.5 mM trisodium citrate. Stringent temperature conditions for the wash steps will ordinarily include a temperature of at least about 25° C., of at least about 42° C., or of at least about 68° C. In certain embodiments, wash steps will occur at 25° C. in 30 mM NaCl, 3 mM trisodium citrate, and 0.1% SDS. In certain embodiments, wash steps will occur at 42° C. in 15 mM NaCl, 1.5 mM trisodium citrate, and 0.1% SDS. In certain embodiments, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Rogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology or identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the)(BLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

By “analog” is meant a structurally related polypeptide or nucleic acid molecule having the function of a reference polypeptide or nucleic acid molecule.

The term “ligand” as used herein refers to a molecule that binds to a receptor. In certain embodiments, the ligand binds to a receptor on another cell, allowing for cell-to-cell recognition and/or interaction.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasm, and pathogen infection of a cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “enforcing tolerance” is meant preventing the activity of self-reactive cells or immunoresponsive cells that target transplanted organs or tissues.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By a “heterologous nucleic acid molecule or polypeptide” is meant a nucleic acid molecule (e.g., a cDNA, DNA or RNA molecule) or polypeptide that is not normally present in a cell or sample obtained from a cell. This nucleic acid may be from another organism, or it may be, for example, an mRNA molecule that is not normally expressed in a cell or sample.

By “modulate” is meant positively or negatively alter. Exemplary modulations include a about 1%, about 2%, about 5%, about 10%, about 25%, about 50%, about 75%, or about 100% change.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

“Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises the amino acid sequence set forth in GGGGSGGGGSGGGGS [SEQ ID NO: 66].

By “neoplasm” is meant a disease characterized by the pathological proliferation of a cell or tissue and its subsequent migration to or invasion of other tissues or organs. Neoplasia growth is typically uncontrolled and progressive, and occurs under conditions that would not elicit, or would cause cessation of, multiplication of normal cells. Neoplasm can affect a variety of cell types, tissues, or organs, including but not limited to an organ selected from the group consisting of bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasia include cancers, such as sarcomas, carcinomas, or plasmacytomas (malignant tumor of the plasma cells).

By “receptor” is meant a polypeptide, or portion thereof, present on a cell membrane that selectively binds one or more ligand.

By “recognize” is meant selectively binds to a target. A T cell that recognizes a tumor can expresses a receptor (e.g., a TCR or CAR) that binds to a tumor antigen.

By “reference” or “control” is meant a standard of comparison. For example, the level of scFv-antigen binding by a cell expressing a CAR and an scFv may be compared to the level of scFv-antigen binding in a corresponding cell expressing CAR alone.

By “secreted” is meant a polypeptide that is released from a cell via the secretory pathway through the endoplasmic reticulum, Golgi apparatus, and as a vesicle that transiently fuses at the cell plasma membrane, releasing the proteins outside of the cell.

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, a human IL-2 signal sequence, e.g., MYRMQLLSCIALSLALVTNS [SEQ ID NO: 67], a mouse IL-2 signal sequence, e.g., MYSMQLASCVTLTLVLLVNS [SEQ ID NO: 68]; a human kappa leader sequence, e.g., METPAQLLFLLLLWLPDTTG [SEQ ID NO: 69], a mouse kappa leader sequence, e.g., METDTLLLWVLLLWVPGSTG [SEQ ID NO: 70]; a human CD8 leader sequence, e.g., MALPVTALLLPLALLLHAARP [SEQ ID NO: 71]; a truncated human CD8 signal peptide, e.g., MALPVTALLLPLALLLHA [SEQ ID NO: 72]; a human albumin signal sequence, e.g., MKWVTFISLLFSSAYS [SEQ ID NO: 73]; and a hman prolactin signal sequence, e.g., MDSKGSSQKGSRLLLLLVVSNLLLCQGVVS [SEQ ID NO: 74]. In certain embodiments, a leader sequence can be an IgG signal peptide or a GM-CSF signal peptide.

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non- IS neoplastic cell. In certain embodiments, a tumor antigen includes any polypeptide expressed by a tumor that is capable of activating or inducing an immune response via a CAR (e.g., CD19, MUC-16) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 7.2. Chimeric Antigen Receptors

The present disclosure provides immunoresponsive cells comprising two or more chimeric antigen receptors (CARs), each of the CARs bind to an antigen, which can be a tumor antigen or a pathogen antigen.

### 7. 2. 1. Antigens

In certain embodiments, at least one of the two or more CARs binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases Erb-B-B2, Erb-B3, Erb-B4, folate-binding protein

(FBP), fetal acetylcholine receptor (AChR), folate receptor-a, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), K-light chain, kinase insert domain receptor (KDR), Lewis Y (LeY), Ll cell adhesion molecule (L1CAM), melanoma antigen family A, 1 (MAGE-A1), Mucin 16 (MUC16), Mucin 1 (MUC1), Mesothelin (MSLN), MAGEA3, p53, MART1,GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR 1 , tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAIVIE, CCR4, CDS, CD3, TRBC1, TRBC2, TIM-3, Integrin B7, ICAM-1, and CLEC12A.

In certain embodiments, one of the two or more CARs binds to a CD19 polypeptide. In certain embodiments, one of the two or more CARs binds to a human CD19 polypeptide. In certain embodiments, the human CD19 polypeptide comprises the amino acid sequence set forth in SEQ ID NO: 75 or a portion thereof.

[SEQ ID NO: 75]
PEEPLVVKVEEGDNAVLQCLKGTSDGPTQQLTWSRESPLKPFLKLSLGLPG
LGIHMRPLAIWLFIENVSQQMGGFYLCQPGPPSEKAWQPGWTVNVEGSGEL
FRWNVSDLGGLGCGLKNRSSEGPSSPSGKLMSPKLYVWAKDRPETWEGEPP
CLPPRDSLNQSLSQDLTMAPGSTLWLSCGVPPDSVSRGPLSWTHVHPKGPK
SLLSLELKDDRPARDMWVMETGLLLPRATAQDAGKYYCHRGNLTMSFHLEI
TARPVLWHWLLRTGGWK

In certain embodiments, one of the two or more CARs binds to the extracellular domain of a CD19 protein.

In certain embodiments, one of the two or more CARs binds to a CD22 polypeptide. In certain embodiments, one of the two or more CARs binds to a human CD22 polypeptide. In certain embodiments, the human CD22 polypeptide comprises the amino acid sequence set forth in SEQ ID NO: 134 or a portion thereof.

[SEQ ID NO: 134]
MHLLGPWLLLLVLEYLAFSDSSKWVFEHPETLYAWEGACVWIPCTYRALDG
DLESFILFHNPEYNKNTSKEDGTRLYESTKDGKVPSEQKRVQFLGDKNKNC
TLSIHPVHLNDSGQLGLRMESKTEKWMERIHLNVSERPFPPHIQLPPEIQE
SQEVTLTCLLNFSCYGYPIQLQWLLEGVPMRQAAVTSTSLTIKSVFTRSEL
KFSPQWSHHGKIVTCQLQDADGKFLSNDTVQLNVKHTPKLEIKVTPSDAIV
REGDSVTMTCEVSSSNPEYTTVSWLKDGTSLKKQNTFTLNLREVTKDQSGK
YCCQVSNDVGPGRSEEVFLQVQYAPEPSTVQILHSPAVEGSQVEFLCMSLA
NPLPTNYTWYHNGKEMQGRTEEKVHIPKILPWHAGTYSCVAENILGTGQRG
PGAELDVQYPPKKVTTVIQNPMPIREGDTVTLSCNYNSSNPSVTRYEWKPH
GAWEEPSLGVLKIQNVGWDNTTIACAACNSWCSWASPVALNVQYAPRDVRV
RKIKPLSEIHSGNSVSLQCDFSSSHPKEVQFFWEKNGRLLGKESQLNFDSI
SPEDAGSYSCWVNNSIGQTASKAWTLEVLYAPRRLRVSMSPGDQVMEGKSA
TLTCESDANPPVSHYTWEDWNNQSLPYHSQKLRLEPVKVQHSGAYWCQGTN
SVGKGRSPLSTLTVYYSPETIGRRVAVGLGSCLAILILAICGLKLQRRWKR
TQSQQGLQENSSGQSFEVRNKKVRRAPLSEGPHSLGCYNPMMEDGISYTTL
REPEMNIPRTGDAESSEMQRPPPDCDDTVTYSALHKRQVGDYENVIPDFPE
DEGIHYSELIQFGVGERPQAQENVDYVILKH

In certain embodiments, one of the two or more CARs binds to the extracellular domain of a CD22 protein.

In certain embodiments, at least one of the two or more CARs binds to a pathogen antigen, e.g., for use in treating and/or preventing a pathogen infection or other infectious disease, for example, in an immunocompromised subject. Non-limiting examples of pathogen includes a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M tuberculosis, M avium, M intracellulare, M kansaii, M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, corynebacterium diphtherias, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 7.2.2. Chimeric Antigen Receptor (CAR)

CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40, CD27, CD40/My88, NKGD2, CD2, CD7, LIGHT, NKG2C, B7-H3, FcεRIγ, TNF receptor proteins, Immunoglobulin-like proteins, cytokine receptors, integrins, signaling lymphocytic activation molecules (SLAM proteins), activating NK cell receptors, BTLA, a Toll ligand receptor, CD30, CDS, ICAM-1, LFA-1 (CD11a/CD18), CDS, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11 a, LFA-1, ITGAM, CD11b, ITGAX, CD11 c, ITGB1, CD29, ITGB2, CD18, LFA-1, ITGB7, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, at least one of the two or more CARs is a second-generation CAR.

In certain embodiments, each of the two or more CARs is a second-generation CAR. In certain embodiments, each of the two or more CARs comprises an extracellular antigen-binding domain that binds to an antigen, a transmembrane domain, and an intracellular signaling domain, wherein the intracellular signaling domain comprises a co-stimulatory signaling region that comprises at least one co-stimulatory molecule or a portion thereof. In certain embodiments, at least one of the two or more CARs further comprises a hinger/spacer region.

In certain embodiments, the extracellular antigen-binding domain of at least one of the two or more CARs (embodied, for example, an scFv or an analog thereof) binds to an antigen with a dissociation constant (K_{d}) of about 2×10⁻⁷ M or less. In certain embodiments, the Ka is about 2×10⁻⁷M or less, about 1×10⁻⁷M or less, about 9×10⁻⁸M or less, about 1×10⁻⁸M or less, about 9×10⁻⁹M or less, about 5×10⁻⁹M or less, about 4×10⁻⁹M or less, about 3×10⁻⁹ or less, about 2×10⁻⁹ M or less, or about 1×10⁻⁹M or less. In certain non-limiting embodiments, the Ka is about 3×10⁻⁹ M or less. In certain non-limiting embodiments, the K_{d}is from about 1×10⁻⁹ M to about 3×10⁻⁷M. In certain non-limiting embodiments, the K_{d}is from about 1.5×10⁻⁹M to about 3×10⁻⁷M. In certain non-limiting embodiments, the K_{d}is from about 1.5×10⁻⁹ M to about 2.7×10⁻⁷M. In certain non-limiting embodiments, the K_{d}is from about 1×10⁻⁴ M to about 1×10⁻⁷M. In certain non-limiting embodiments, the K_{d}is from about 1×10⁻¹³ M to about 1×10⁻¹⁵M.

Binding of the extracellular antigen-binding domain (for example, in an scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalamal), cyan fluorescent protein (e.g., ECFP, Cerulean, and CyPet), and yellow fluorescent protein (e.g., YFP, Citrine, Venus, and YPet).

### 7. 2. 2. 1. Extracellular Antigen Binding Domain of a CAR

In certain embodiments, the extracellular antigen-binding domain of at least one of the two or more CARs specifically binds to an antigen. In certain embodiments, the extracellular antigen-binding domain of at least one of the two or more CARs is an scFv. In certain embodiments, the scFv is a human scFv. In certain embodiments, the scFv is a humanized scFv. In certain embodiments, the scFv is a non-human (e.g., murine, rabit, rat, etc.) scFv. In certain embodiments, the scFv is a murine scFv. In certain embodiments, the extracellular antigen-binding domain of at least one of the two or more CARs is a Fab, which is optionally crosslinked. In certain embodiments, the extracellular antigen-binding domain of at least one of the two or more CARs is a F(ab)₂. In certain embodiments, any of the foregoing molecules may be comprised in a fusion protein with a heterologous sequence to form the extracellular antigen-binding domain. In certain embodiments, the scFv is identified by screening scFv phage library with an antigen-Fc fusion protein. The scFv can be derived from a mouse bearing human light chain variable region (“V_{L}”) and/or heavy chain variable region (“V_{H}”) genes. The scFv can also be substituted with a camelid Heavy chain (e.g., VHH, from camel, lama, etc.) or a partial natural ligand for a cell surface receptor.

In certain embodiments, the extracellular antigen-binding domain of one of the two or more CARs is a murine scFv. In certain embodiments, the extracellular antigen-binding domain of one of the two or more CARs is a murine scFv that binds to a human CD19 polypeptide. In certain embodiments, the extracellular antigen-binding domain of one of the two or more CARs is a murine scFv, which comprises the amino acid sequence set forth in SEQ ID NO: 84 and specifically binds to a human CD19 polypeptide (e.g., a human CD19 polypeptide comprising the amino acid sequence set forth in SEQ ID NO: 75 or a portion thereof). SEQ ID NO: 84 is provided in Table 1.

An exemplary nucleotide sequence encoding the amino acid sequence of SEQ ID NO: 84 is set forth in SEQ ID NO: 85. SEQ ID NO: 85 is provided in Table 1.

In certain embodiments, the extracellular antigen-binding domain of one of the two or more CARs comprises a V_{H} comprising an amino acid sequence that is at least about 80% (e.g., at least about 85%, at least about 90%, or at least about 95%) homologous or identical to SEQ ID NO: 82. For example, the extracellular antigen-binding domain of one of the two or more CARs comprises a V_{H} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 82. In certain embodiments, the extracellular antigen-binding domain of one of the two or more CARs comprises a V_{H} comprising the amino sequence set forth in SEQ ID NO: 82. In certain embodiments, the extracellular antigen-binding domain of one of the two or more CARs comprises a V_{L} comprising an amino acid sequence that is at least about 80% (e.g., at least about 85%, at least about 90%, or at least about 95%) homologous or identical to SEQ ID NO: 83. For example, the extracellular antigen-binding domain of one of the two or more CARs comprises a V_{L} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 83. In certain embodiments, the extracellular antigen-binding domain of one of the two or more CARs comprises a V_{L} comprising the amino acid sequence set forth in SEQ ID NO: 83. In certain embodiments, the extracellular antigen-binding domain of one of the two or more CARs comprises a V_{H} comprising an amino acid sequence that is at least about 80% (e.g., at least about 85%, at least about 90%, or at least about 95%) homologous or identical to SEQ ID NO: 82, and a V_{L} comprising an amino acid sequence that is at least about 80% (e.g., at least about 85%, at least about 90%, or at least about 95%) homologous or identical to SEQ ID NO: 83. In certain embodiments, the extracellular antigen-binding domain of one of the two or more CARs comprises a V_{H} comprising the amino acid sequence set forth in SEQ ID NO: 82 and a V_{L} comprising the amino acid sequence set forth in SEQ ID NO: 83. In certain embodiments, the extracellular antigen-binding domain of one of the two or more CARs comprises a V_{H} comprising the amino acid sequence set forth in SEQ ID NO: 82 and a V_{L} comprising the amino acid sequence set forth in SEQ ID NO: 83, and a linker positioned between the V_{H} and the V_{L}. In certain embodiments, the linker comprises the amino acid sequence set forth in SEQ ID NO: 66. SEQ ID NOs: 82 and 83 are provided in Table 1.

In certain embodiments, the extracellular antigen-binding domain of one of the two or more CARs comprises a V_{H} CDR1 comprising the amino acid sequence set forth in SEQ ID NO: 76, or a conservative modification thereof, a V_{H} CDR2 comprising the amino acid sequence set forth in SEQ ID NO: 77 or a conservative modification thereof, and a V_{H} CDR3 comprising the amino acid sequence set forth in SEQ ID NO: 78, a conservative modification thereof. In certain embodiments, the extracellular antigen-binding domain of one of the two or more CARs comprises a V_{H} CDR1 comprising the amino acid sequence set forth in SEQ ID NO: 76, a V_{H} CDR2 comprising the amino acid sequence set forth in SEQ ID NO: 77, and a V_{H} CDR3 comprising the amino acid sequence set forth in SEQ ID NO: 78.

In certain embodiments, the extracellular antigen-binding domain of one of the two or more CARs comprises a V_{L} CDR1 comprising the amino acid sequence set forth in SEQ ID NO: 79 or a conservative modification thereof, a V_{L} CDR2 comprising the amino acid sequence set forth in SEQ ID NO: 80 or a conservative modification thereof, and a V_{L} CDR3 comprising the amino acid sequence set forth in SEQ ID NO: 81 or a conservative modification thereof. In certain embodiments, the extracellular antigen-binding domain of one of the two or more CARs comprises a V_{L} CDR1 comprising the amino acid sequence set forth in SEQ ID NO: 79, a V_{L} CDR2 comprising the amino acid sequence set forth in SEQ ID NO: 80, and a V_{L} CDR3 comprising the amino acid sequence set forth in SEQ ID NO: 81.

In certain embodiments, the extracellular antigen-binding domain of one of the two or more CARs comprises a V_{H} CDR1 comprising the amino acid sequence set forth in SEQ ID NO: 76 or a conservative modification thereof, a V_{H} CDR2 comprising the amino acid sequence set forth in SEQ ID NO: 77 or a conservative modification thereof, a V_{H} CDR3 comprising the amino acid sequence set forth in SEQ ID NO: 78, a conservative modification thereof, a V_{L} CDR1 comprising the amino acid sequence set forth in SEQ ID NO: 79 or a conservative modification thereof, a V_{L} CDR2 comprising the amino acid sequence set forth in SEQ ID NO: 80 or a conservative modification thereof, and a V_{L} CDR3 comprising the amino acid sequence set forth in SEQ ID NO: 81 or a conservative modification thereof In certain embodiments, the extracellular antigen-binding domain of one of the two or more CARs comprises a V_{H} CDR1 comprising amino acids having the sequence set forth in SEQ ID NO: 76, a V_{H} CDR2 comprising the amino acid sequence set forth in SEQ ID NO: 77, a V_{H} CDR3 comprising the amino acid sequence set forth in SEQ ID NO: 78, a V_{L} CDR1 comprising the amino acid sequence set forth in SEQ ID NO: 79, a V_{L} CDR2 comprising the amino acid sequence set forth in SEQ ID NO: 80 and a V_{L} CDR3 comprising the amino acid sequence set forth in SEQ ID NO: 81. SEQ ID NOs: 76-81 are provided in Table 1.

TABLE 1

| | | anti-human CD19 scFv (SJ25C1) | | |
|---|---|---|---|---|
| CDRs | | 1 | 2 | 3 |
| V_{H} | a.a. | GYAFSS [SEQ ID NO: | YPGDGD [SEQ ID NO: 77] | KTISSVVDF [SEQ |
| | | 76] | ID NO: 78] | |
| V_{L} | a.a. | KASQNVGTNVA [SEQ ID | SATYRN [SEQ ID NO: 80] | QQYNRYPYT [SEQ |
| | | NO: 79] | | ID NO: 81] |

| | |
|---|---|
| Full V_{H} | EVKLQQSGAE LVRPGSSVKI SCKASGYAFS SYWMNWVKQR PGQGLEWIGQ |
| | IYPGDGDTNY NGKFKGQATL TADKSSSTAY MQLSGLTSED SAVYFCARKT |
| | ISSVVDFYFD YWGQGTTVTV SS [SEQ ID NO: 82] |
| Full V_{L} | DIELTQSPKF MSTSVGDRVS VTCKASQNVG TNVAWYQQKP GQSPKPLIYS |
| | ATYRNSGVPD RFTGSGSGTD FTLTITNVQS KDLADYFCQQ YNRYPYTSGG GTKLEIKR |
| | [SEQ ID NO: 83] |
| scFv | MALPVTALLL PLALLLHAEV KLQQSGAELV RPGSSVKISC KASGYAFSSY |
| | WMNWVKQRPG QGLEWIGQIY PGDGDTNYNG KFKGQATLTA DKSSSTAYMQ |
| | LSGLTSEDSA VYFCARKTIS SVVDFYFDYW GQGTTVTVSS GGGGSGGGGS |
| | GGGGSDIELT QSPKFMSTSV GDRVSVTCKA SQNVGTNVAW YQQKPGQSPK |
| | PLIYSATYRN SGVPDRFTGS GSGTDFTLTI TNVQSKDLAD YFCQQYNRYP |
| | YTSGGGTKLE IKR [SEQ ID NO: 84] |
| DNA | ATGGCTCTCCCAGTGACTGCCCTACTGCTTCCCCTAGCGCTTCTCCTGCATGCAGAGGTGAAG |
| | CTGCAGCAGTCTGGGGCTGAGCTGGTGAGGCCTGGGTCCTCAGTGAAGATTTCCTGCAAGGCT |
| | TCTGGCTATGCATTCAGTAGCTACTGGATGAACTGGGTGAAGCAGAGGCCTGGACAGGGTCTT |
| | GAGTGGATTGGACAGATTTATCCTGGAGATGGTGATACTAACTACAATGGAAAGTTCAAGGGT |
| | CAAGCCACACTGACTGCAGACAAATCCTCCAGCACAGCCTACATGCAGCTCAGCGGCCTAACA |
| | TCTGAGGACTCTGCGGTCTATTTCTGTGCAAGAAAGACCATTAGTTCGGTAGTAGATTTCTAC |
| | TTTGACTACTGGGGCCAAGGGACCACGGTCACCGTCTCCTCAGGTGGAGGTGGATCAGGTGGA |
| | GGTGGATCTGGTGGAGGTGGATCTGACATTGAGCTCACCCAGTCTCCAAAATTCATGTCCACA |
| | TCAGTAGGAGACAGGGTCAGCGTCACCTGCAAGGCCAGTCAGAATGTGGGTACTAATGTAGCC |
| | TGGTATCAACAGAAACCAGGACAATCTCCTAAACCACTGATTTACTCGGCAACCTACCGGAAC |
| | AGTGGAGTCCCTGATCGCTTCACAGGCAGTGGATCTGGGACAGATTTCACTCTCACCATCACT |
| | AACGTGCAGTCTAAAGACTTGGCAGACTATTTCTGTCAACAATATAACAGGTATCCGTACACG |
| | TCCGGAGGGGGGACCAAGCTGGAGATCAAACGG [SEQ ID NO: 85] |

In certain embodiments, the extracellular antigen-binding domain of one of the two or more CARs comprises a V_{H} comprising an amino acid sequence that is at least about 80% (e.g., at least about 85%, at least about 90%, or at least about 95%) homologous or identical to SEQ ID NO: 143. For example, the extracellular antigen-binding domain of one of the two or more CARs comprises a V_{H} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% homologous or identical to SEQ ID NO: 143. In certain embodiments, the extracellular antigen-binding domain of one of the two or more CARs comprises a V_{H} comprising the amino sequence set forth in SEQ ID NO: 143. In certain embodiments, the extracellular antigen-binding domain of one of the two or more CARs comprises a V_{L} comprising an amino acid sequence that is at least about 80% (e.g., at least about 85%, at least about 90%, or at least about 95%) homologous or identical to SEQ ID NO: 144. For example, the extracellular antigen-binding domain of one of the two or more CARs comprises a V_{L} comprising an amino acid sequence that is at least about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 144. In certain embodiments, the extracellular antigen-binding domain of one of the two or more CARs comprises a V_{L} comprising the amino acid sequence set forth in SEQ ID NO: 144. In certain embodiments, the extracellular antigen-binding domain of one of the two or more CARs comprises a V_{H} comprising an amino acid sequence that is at least about 80% (e.g., at least about 85%, at least about 90%, or at least about 95%) homologous or identical to SEQ ID NO: 143, and a V_{L} comprising an amino acid sequence that is at least about 80% (e.g., at least about 85%, at least about 90%, or at least about 95%) homologous or identical to SEQ ID NO: 144. In certain embodiments, the extracellular antigen-binding domain of one of the two or more CARs comprises a V_{H} comprising the amino acid sequence set forth in SEQ ID NO: 143 and a V_{L} comprising the amino acid sequence set forth in SEQ ID NO: 144. In certain embodiments, the extracellular antigen-binding domain of one of the two or more CARs comprises a V_{H} comprising the amino acid sequence set forth in SEQ ID NO: 143 and a V_{L} comprising the amino acid sequence set forth in SEQ ID NO: 144 , and a linker positioned between the V_{H} and the V_{L}. In certain embodiments, the linker comprises the amino acid sequence set forth in SEQ ID NO: 66. SEQ ID NOs: 143 and 144 are provided in Table 2.

An exemplary nucleotide sequence encoding the amino acid sequence of SEQ ID NO: 143 is set forth in SEQ ID NO: 145. An exemplary nucleotide sequence encoding the amino acid sequence of SEQ ID NO: 144 is set forth in SEQ ID NO: 146. SEQ ID NOs: 145 and 146 are provided in Table 2.

In certain embodiments, the extracellular antigen-binding domain of one of the two or more CARs comprises a V_{H} CDR1 comprising the amino acid sequence set forth in SEQ ID NO: 124, or a conservative modification thereof, a V_{H} CDR2 comprising the amino acid sequence set forth in SEQ ID NO: 125 or a conservative modification thereof, and a V_{H} CDR3 comprising the amino acid sequence set forth in SEQ ID NO: 126, a conservative modification thereof. In certain embodiments, the extracellular antigen-binding domain of one of the two or more CARs comprises a V_{H} CDR1 comprising the amino acid sequence set forth in SEQ ID NO: 124, a V_{H} CDR2 comprising the amino acid sequence set forth in SEQ ID NO: 125, and a V_{H} CDR3 comprising the amino acid sequence set forth in SEQ ID NO: 126. In certain embodiments, the extracellular antigen-binding domain of one of the two or more CARs comprises a V_{L} CDR1 comprising the amino acid sequence set forth in SEQ ID NO: 127 or a conservative modification thereof, a V_{L} CDR2 comprising the amino acid sequence set forth in SEQ ID NO: 128 or a conservative modification thereof, and a V_{L} CDR3 comprising the amino acid sequence set forth in SEQ ID NO: 129 or a conservative modification thereof. In certain embodiments, the extracellular antigen-binding domain of one of the two or more CARs comprises a V_{L} CDR1 comprising the amino acid sequence set forth in SEQ ID NO: 127, a V_{L} CDR2 comprising the amino acid sequence set forth in SEQ ID NO: 128, and a V_{L} CDR3 comprising the amino acid sequence set forth in SEQ ID NO: 129. In certain embodiments, the extracellular antigen-binding domain of one of the two or more CARs comprises a V_{H} CDR1 comprising the amino acid sequence set forth in SEQ ID NO: 124 or a conservative modification thereof, a V_{H} CDR2 comprising the amino acid sequence set forth in SEQ ID NO: 125 or a conservative modification thereof, a V_{H} CDR3 comprising the amino acid sequence set forth in SEQ ID NO: 126, a conservative modification thereof, a V_{L} CDR1 comprising the amino acid sequence set forth in SEQ ID NO: 127 or a conservative modification thereof, a V_{L} CDR2 comprising the amino acid sequence set forth in SEQ ID NO: 128 or a conservative modification thereof, and a V_{L} CDR3 comprising the amino acid sequence set forth in SEQ ID NO: 129 or a conservative modification thereof In certain embodiments, the extracellular antigen-binding domain of one of the two or more CARs comprises a V_{H} CDR1 comprising amino acids having the sequence set forth in SEQ ID NO: 124, a V_{H} CDR2 comprising the amino acid sequence set forth in SEQ ID NO: 125, a V_{H} CDR3 comprising the amino acid sequence set forth in SEQ ID NO: 126, a V_{L} CDR1 comprising the amino acid sequence set forth in SEQ ID NO: 127, a V_{L} CDR2 comprising the amino acid sequence set forth in SEQ ID NO: 128 and a V_{L} CDR3 comprising the amino acid sequence set forth in SEQ ID NO: 129. SEQ ID NOs: 124-129 are provided in Table 2.

TABLE 2

| | | anti-human CD19 scFv (FMC63) | | |
|---|---|---|---|---|
| CDRs | | 1 | 2 | 3 |
| V_{H} | a.a. | GVSLPDY [SEQ ID NO: | WGSET [SEQ ID NO: 125] | HYYYGGSYAMDY |
| | | 124] | | [SEQ ID NO: |
| | | | | 126] |
| V_{L} | a.a. | RASQDISKYLN [SEQ ID | HTSRLHS [SEQ ID NO: | QQGNTLPYT |
| | | NO: 127] | 128] | [SEQ ID NO: |
| | | | | 129] |

| | |
|---|---|
| Full V_{H} | EVKLQESGPGLVAPSQSLSVTCTVSGVSLPDYGVSWIRQPPRKGLEWLGVIWGSETTYYNS |
| | ALKSRLTIIKDNSKSQVFLKMNSLQTDDTAIYYCAKHYYYGGSYAMDYWGQGTSVTVSS |
| | [SEQ ID NO: 143] |
| Full V_{L} | DIQMTQTTSSLSASLGDRVTISCRASQDISKYLNWYQQKPDGTVKLLIYHTSRLHSGVPSR |
| | FSGSGSGTDYSLTISNLEQEDIATYFCQQGNTLPYTEGGGTKLEIT [SEQ ID NO: |
| | 144] |
| Full V_{H} | gaggtgaaactgcaggagtcaggacctggcctggtggcgccctcacagagcctgtccgtca |
| DNA | catgcactgtctcaggggtctcattacccgactatggtgtaagctggattcgccagcctcc |
| | acgaaagggtctggagtggctgggagtaatatggggtagtgaaaccacatactataattca |
| | gctctcaaatccagactgaccatcatcaaggacaactccaagagccaagttttcttaaaaa |
| | tgaacagtctgcaaactgatgacacagccatttactactgtgccaaacattattactacgg |
| | tggtagctatgctatggactactggggccaaggaacctcagtcaccgtctcctca [SEQ |
| | ID NO: 145] |
| Full V_{L} | gacatccagatgacacagactacatcctccctgtctgcctctctgggagacagagtcacca |
| DNA | tcagttgcagggcaagtcaggacattagtaaatatttaaattggtatcagcagaaaccaga |
| | tggaactgttaaactcctgatctaccatacatcaagattacactcaggagtcccatcaagg |
| | ttcagtggcagtgggtctggaacagattattctctcaccattagcaacctggagcaagaag |
| | atattgccacttacttttgccaacagggtaatacgcttccgtacacgttcggaggggggac |
| | caagctggagatcac [SEQ ID NO: 146] |

In certain embodiments, the extracellular antigen-binding domain of one of the two or more CARs is a human scFv that binds to a human CD22 polypeptide. In certain embodiments, the extracellular antigen-binding domain of one of the two or more CARs is a human scFv, which comprises the amino acid sequence of SEQ ID NO: 132 and specifically binds to a human CD22 polypeptide (e.g., a human CD22 polypeptide comprising the amino acid sequence set forth in SEQ ID NO: 134 or a portion thereof). An exemplary nucleotide sequence encoding the amino acid sequence of SEQ ID NO: 132 is set forth in SEQ ID NO: 133. SEQ ID NOs: 132 and 133 are provided in Table 3.

In certain embodiments, the extracellular antigen-binding domain of one of the two or more CARs comprises a V_{H} comprising an amino acid sequence that is at least about 80% (e.g., at least about 85%, at least about 90%, or at least about 95%) homologous to SEQ ID NO: 130. For example, the extracellular antigen-binding domain of one of the two or more CARs comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 130. In certain embodiments, the extracellular antigen-binding domain of one of the two or more CARs comprises a V_{H} comprising the amino sequence set forth in SEQ ID NO: 130. In certain embodiments, the extracellular antigen-binding domain of one of the two or more CARs comprises a V_{L} comprising an amino acid sequence that is at least about 80% (e.g., at least about 85%, at least about 90%, or at least about 95%) homologous or identical to SEQ ID NO: 131. For example, the extracellular antigen-binding domain of one of the two or more CARs comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to SEQ ID NO: 131. In certain embodiments, the extracellular antigen-binding domain of one of the two or more CARs comprises a V_{L} comprising the amino acid sequence set forth in SEQ ID NO: 131. In certain embodiments, the extracellular antigen-binding domain of one of the two or more CARs comprises a V_{H} comprising an amino acid sequence that is at least about 80% (e.g., at least about 85%, at least about 90%, or at least about 95%) homologous or identical to SEQ ID NO: 130, and a V_{L} comprising an amino acid sequence that is at least about 80% (e.g., at least about 85%, at least about 90%, or at least about 95%) homologous or identical to SEQ ID NO: 131. In certain embodiments, the extracellular antigen-binding domain of one of the two or more CARs comprises a V_{H} comprising the amino acid sequence set forth in SEQ ID NO: 130 and a V_{L} comprising the amino acid sequence set forth in SEQ ID NO: 131. In certain mbodiments, the extracellular antigen-binding domain of one of the two or more CARs comprises a V_{H} comprising the amino acid sequence set forth in SEQ ID NO: 130 and a V_{L} comprising the amino acid sequence set forth in SEQ ID NO: 131, and a linker positioned between the V_{H} and the V_{L}. In certain embodiments, the linker comprises amino acids having the sequence set forth in SEQ ID NO: 66. SEQ ID NOs: 130 and 131 are provided in Table 3.

TABLE 3
anti-human CD22 scFv

| | |
|---|---|
| Full V_{H} | QVQLQQSGPGLVKPSQTLSLTCAISGDSVSSNSAAWNWIRQSPSRGLEWLGRTYYRSKWYN |
| | DYAVSVKSRITINPDTSKNQFSLQLNSVTPEDTAVYYCAREVTGDLEDAFDIWGQGTMVTV |
| | SS [SEQ ID NO: 130] |
| Full V_{L} | DIQMTQSPSSLSASVGDRVTITCRASQTIWSYLNWYQQRPGKAPNLLIYAASSLQSGVPSR |
| | FSGRGSGTDFTLTISSLQAEDFATYYCQQSYSIPQTFGQGTKLEIK [SEQ ID NO: |
| | 131] |
| scFv | MELGLSWIFLLAILKGVQCQVQLQQSGPGLVKPSQTLSLTCAISGDSVSSNSAAWNWIRQS |
| | PSRGLEWLGRTYYRSKWYNDYAVSVKSRITINPDTSKNQFSLQLNSVTPEDTAVYYCAREV |
| | TGDLEDAFDIWGQGTMVTVSSGGGGSDIQMTQSPSSLSASVGDRVTITCRASQTIWSYLNW |
| | YQQRPGKAPNLLIYAASSLQSGVPSRFSGRGSGTDFTLTISSLQAEDFATYYCQQSYSIPQ |
| | TFGQGTKLEIK [SEQ ID NO: 132] |
| DNA | aTGGAACTCGGTCTGTCCTGGATTTTTCTGCTTGCGATCTTGAAAGGAGTGCAGTGCCAAG |
| | TACAGCTTCAACAGTCAGGTCCGGGGCTGGTAAAGCCTTCTCAGACACTCAGCCTGACTTG |
| | TGCTATAAGTGGGGATAGTGTTTCATCCAACTCAGCGGCGTGGAACTGGATCCGGCAAAGT |
| | CCATCTCGGGGCCTGGAGTGGTTGGGGCGGACCTATTATAGGTCTAAGTGGTACAATGATT |
| | ACGCCGTCTCAGTGAAGTCACGGATCACAATCAATCCCGATACGAGTAAGAATCAGTTCTC |
| | ACTTCAGCTTAACAGTGTGACACCTGAAGATACGGCAGTATATTATTGCGCGAGAGAGGTT |
| | ACTGGGGACCTCGAAGATGCCTTCGATATCTGGGGTCAAGGCACAATGGTTACAGTCAGCT |
| | CCGGAGGAGGAGGCAGCGACATACAGATGACACAATCTCCGAGTAGCCTTTCCGCATCCGT |
| | AGGTGATAGGGTTACCATAACTTGCCGCGCATCTCAAACGATCTGGTCCTATCTGAACTGG |
| | TACCAGCAGAGACCAGGAAAAGCTCCTAATCTGCTTATCTACGCCGCAAGCTCACTGCAGT |
| | CTGGGGTTCCGAGTAGATTTTCTGGGCGAGGCAGCGGAACGGATTTTACTCTGACCATAAG |
| | CTCTCTGCAAGCAGAAGATTTTGCCACGTACTACTGCCAGCAATCTTACAGCATCCCACAA |
| | ACATTTGGACAAGGCACAAAGTTGGAGATCAAA [SEQ ID NO: 133] |

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

In certain embodiments, the V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NO: 82, SEQ ID NO: 83, SEQ ID NO: 130, SEQ ID NO: 131, SEQ ID NO: 143, or SEQ ID NO: 144) may contain substitutions (e.g., conservative substitutions), insertions, or deletions relative to the specified sequence(s), but retain the ability to bind to a target antigen (e.g., CD19 or CD22). In certain embodiments, a total of 1 to 10 amino acids are substituted, inserted and/or deleted in a specific sequence (e.g., SEQ ID NO: 82, SEQ ID NO: 83, SEQ ID NO: 130, SEQ ID NO: 131, SEQ ID NO: 143, or SEQ ID NO: 144). In certain embodiments, substitutions, insertions, or deletions occur in regions outside the CDRs (e.g., in the FRs) of the extracellular antigen-binding domain.

### 7.2.2.2. Transmembrane Domain of a CAR

In certain embodiments, the transmembrane domain of each of the two or more CARs comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of each of the two or more CARs can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 polypeptide, a NKGD2 polypeptide, a CD2 polypeptide, a CD7 polypeptide, a LIGHT polypeptide, a NKG2C polypeptide, a B7-H3 polypeptide, a FcεRIγ polypeptide, a TNF receptor polypeptide, an Immunoglobulin-like polypeptide, a cytokine polypeptide, an integrin polypeptide, a signaling lymphocytic activation molecule polypeptide (a SLAM polypeptide), an activating NK cell receptor polypeptide, a BTLA polypeptide, a Toll ligand receptor polypeptide, a CD30 polypeptide, a CDS polypeptide, an ICAM-1 polypeptide, a LFA-1 (CD11a/CD18) polypeptide, a CDS polypeptide, a GITR polypeptide, a BAFFR polypeptide, a HVEM (LIGHTR) polypeptide, a KIRDS2 polypeptide, a SLAMF7 polypeptide, a NKp80 (KLRF1) polypeptide, a NKp44 polypeptide, a NKp30 polypeptide, a NKp46 polypeptide, a CD19 polypeptide, a CD4 polypeptide, a CD8alpha polypeptide, a CD8beta polypeptide, an IL2R beta polypeptide, an IL2R gamma polypeptide, an IL7R alpha polypeptide, an ITGA4 polypeptide, a VLA1 polypeptide, a CD49a polypeptide, an ITGA4 polypeptide, an IA4 polypeptide, a CD49D polypeptide, an ITGA6 polypeptide, a VLA-6 polypeptide, a CD49f polypeptide, an ITGAD polypeptide, a CD11d polypeptide, an ITGAE polypeptide, a CD103 polypeptide, an ITGAL polypeptide, a CDlla polypeptide, a LFA-1 polypeptide, an ITGAM polypeptide, a CDllb polypeptide, an ITGAX polypeptide, a CD11c polypeptide, an ITGB1 polypeptide, a CD29 polypeptide, an ITGB2 polypeptide, a CD18 polypeptide, a LFA-1 polypeptide, an ITGB7 polypeptide, a NKG2C polypeptide, a TNFR2 polypeptide, a TRANCE/RANKL polypeptide, a DNAM1 (CD226) polypeptide, a SLAMF4 (CD244, 2B4) polypeptide, a CD84 polypeptide, a CD96 (Tactile) polypeptide, a CEACAM1 polypeptide, a CRTAM polypeptide, a Ly9 (CD229) polypeptide, a CD160 (BY55) polypeptide, a PSGL1 polypeptide, a CD100 (SEMA4D) polypeptide, a CD69 polypeptide, a SLAMF6 (NTB-A, Ly108) polypeptide, a SLAM (SLAMF1, CD150, IPO-3) polypeptide, a BLAME (SLAMF8) polypeptide, a SELPLG (CD162) polypeptide, a LTBR polypeptide, a LAT polypeptide, a GADS polypeptide, a SLP-76 polypeptide, a PAG/Cbp polypeptide, a CD19a polypeptide, and a ligand that specifically binds with CD83, a synthetic polypeptide (not based on a protein associated with the immune response), or a combination thereof.

CD8

In certain embodiments, the transmembrane domain of at least one of the two or more CARs comprises a CD8 polypeptide, e.g., a transmembrane domain of CD8 or a portion thereof In certain embodiments, the transmembrane domain of at least one of the two or more CARs comprises a transmembrane domain of human CD8 or a portion thereof In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001139345.1 (SEQ ID NO: 86) as provided below, or a fragment thereof, and/or may optionally comprise up to one or up to two or up to three conservative amino acid substitutions. In certain embodiments, the CD8 polypeptide comprises or has an amino acid sequence that is a consecutive portion of SEQ ID NO: 86, which is at least about 20, or at least about 30, or at least about 40, or at least about 50, at least about 70, and up to 235 amino acids in length. Alternatively or additionally, in non-limiting various embodiments, the CD8 polypeptide comprises or has an amino acid sequence of amino acids 1 to 235, 1 to 50, 50 to 100, 100 to 150, 137 to 209, 150 to 200, or 200 to 235 of SEQ ID NO: 86. In certain embodiments, the CD8 polypeptide comprises or has amino acids 137 to 209 of SEQ ID NO: 86.

[SEQ ID NO: 86]
MALPVTALLLPLALLLHAARPSQFRVSPLDRTWNLGETVELKCQVLLSNPT
SGCSWLFQPRGAAASPTFLLYLSQNKPKAAEGLDTQRFSGKRLGDTFVLTL
SDFRRENEGYYFCSALSNSIMYFSHFVPVFLPAKPTTTPAPRPPTPAPTIA
SQPLSLRPEACRPAAGGAVHTRGLDFACDIYIWAPLAGTCGVLLLSLVITL
YCNHRNRRRVCKCPRPVVKSGDKPSLSARYV

In certain embodiments, the transmembrane domain of at least one of the two or more CARs comprises a transmembrane domain of mouse CD8 or a portion thereof. In certain embodiments, the CD8 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: AAA92533.1 (SEQ ID NO: 87) as provided below, or a fragment thereof, and/or may optionally comprise up to one or up to two or up to three conservative amino acid substitutions. In certain embodiments, the CD8 polypeptide comprises or has an amino acid sequence that is a consecutive portion of SEQ ID NO: 87, which is at least about 20, or at least about 30, or at least about 40, or at least about 50, or at least about 60, or at least about 70, or at least about 100, or at least about 200, and up to 247 amino acids in length. Alternatively or additionally, in non-limiting various embodiments, the CD8 polypeptide comprises or has an amino acid sequence of amino acids 1 to 247, 1 to 50, 50 to 100, 100 to 150, 150 to 200, 151 to 219, or 200 to 247 of SEQ ID NO: 87. In certain embodiments, the CD8 polypeptide comprises or has amino acids 151 to 219 of SEQ ID NO: 87.

[SEQ ID NO: 87]

| | |
|---|---|
| 1 | MASPLTRELS LNLLLMGESI ILGSGEAKPQ APELRIFPKK |
| | MDAELGQKVD LVCEVLGSVS |
| 61 | QGCSWLFQNS SSKLPQPTFV VYMASSHNKI TWDEKLNSSK |
| | LFSAVRDTNN KYVLTLNKFS |
| 121 | KENEGYYFCS VISNSVMYFS SVVPVLQKVN STTTKPVLRT |
| | PSPVHPTGTS QPQRPEDCRP |
| 181 | RGSVKGTGLD FACDIYIWAP LAGICVAPLL SLIITLICYH |
| | RSRKRVCKCP RPLVRQEGKP |
| 241 | RPSEKIV |

In certain embodiments, the CD8 polypeptide comprises or has the amino acid sequence set forth in SEQ ID NO: 88, which is provided below:

[SEQ ID NO: 88]
STTTKPVLRTPSPVHPTGTSQPQRPEDCRPRGSVKGTGLDFACDIYIWAPL
AGICVALLLSLITTLICY

In accordance with the presently disclosed subject matter, a “CD8 nucleic acid molecule” refers to a polynucleotide encoding a CD8 polypeptide.

An exemplary nucleotide sequence encoding the amino acid sequence of SEQ ID NO: 88 is set forth in SEQ ID NO: 89, which is provided below.

[SEQ ID NO: 89]
TCTACTACTACCAAGCCAGTGCTGCGAACTCCCTCACCTGTGCACCCTACC
GGGACATCTCAGCCCCAGAGACCAGAAGATTGTCGGCCCCGTGGCTCAGTG
AAGGGGACCGGATTGGACTTCGCCTGTGATATTTACATCTGGGCACCCTTG
GCCGGAATCTGCGTGGCCCTTCTGCTGTCCTTGATCATCACTCTCATCTGC
TAC

CD28

In certain embodiments, the transmembrane domain of at least one of the two or more CARs comprises a CD28 polypeptide, e.g., a transmembrane domain of CD28 or a portion thereof. In certain embodiments, the transmembrane domain of at least one of the two or more CARs comprises a transmembrane domain of human CD28 or a portion thereof. In certain embodiments, the CD28 polypeptide comprises or has an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_006130 (SEQ ID NO: 90), or a fragment thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 90, which is at least about 20, or at least about 30, or at least about 40, or at least about 50, and up to 220 amino acids in length. Alternatively or additionally, in non-limiting various embodiments, the CD28 polypeptide comprises or has an amino acid sequence of amino acids 1 to 220, 1 to 50, 50 to 100, 100 to 150, 150 to 200, 153 to 179, or 200 to 220 of SEQ ID NO: 90. In certain embodiments, the CD28 polypeptide comprised in the transmembrane domain of at least one of the two or more CARs comprises or has amino acids 153 to 179 of SEQ ID NO: 90. SEQ ID NO: 90 is provided below:

[SEQ ID NO: 90]

| | |
|---|---|
| 1 | MLRLLLALNL FPSIQVTGNK ILVKQSPMLV AYDNAVNLSC |
| | KYSYNLFSRE FRASLHKGLD |
| 61 | SAVEVCVVYG NYSQQLQVYS KTGFNCDGKL GNESVTFYLQ |
| | NLYVNQTDIY FCKIEVMYPP |
| 121 | PYLDNEKSNG TIIHVKGKHL CPSPLFPGPS KPFWVLVVVG |
| | GVLACYSLLV TVAFIIFWVR |
| 181 | SKRSRLLHSD YMNMTPRRPG PTRKHYQPYA PPRDFAAYRS |

In accordance with the presently disclosed subject matter, a “CD28 nucleic acid molecule” refers to a polynucleotide encoding a CD28 polypeptide. An exemplary nucleotide sequence encoding the amino acids 153 to 179 of SEQ ID NO: 90 is set forth in SEQ ID NO: 91, which is provided below.

[SEQ ID NO: 91]
ttttgggtgctggtggtggttggtggagtcctggcttgctatagcttgcta
gtaacagtggcctttattattttctgggtg

In certain embodiments, the transmembrane domain of the CAR comprises a transmembrane domain of human CD28 or a portion thereof. In certain embodiments, the transmembrane domain of human CD28 or a portion thereof comprises or has an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 92 or a fragment thereof, and/or may optionally comprise up to one or up to two or up to three conservative amino acid substitutions. SEQ ID NO: 92 is provided below:

[SEQ ID NO: 92]
FWVLVVVGGV LACYSLLVTV AFIIFWV.

An exemplary nucleotide sequence encoding the amino acid sequence of SEQ ID NO: 92 is set forth in SEQ ID NO: 93, which is provided below.

[SEQ ID NO: 93]
TTTTGGGTGCTGGTGGTGGTTGGTGGAGTCCTGGCTTGCTATAGCTTGCTA
GTAACAGTGGCCTTTATTATTTTCTGGGTG

CD84

In certain embodiments, the transmembrane domain of at least one of the two or more CARs comprises a native or modified transmembrane domain of a CD84 polypeptide or a portion thereof. The CD84 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001171808.1 (SEQ ID No: 1), or a fragment thereof, and/or may optionally comprise up to one or up to two or up to three conservative amino acid substitutions. In non-limiting certain embodiments, the CD84 polypeptide comprises or has an amino acid sequence that is a consecutive portion of SEQ ID NO: 1, which is at least about 20 (e.g., about 25), or at least about 30, or at least about 40, or at least about 50, or at least about 100, and up to about 345 amino acids in length. Alternatively or additionally, in non-limiting various embodiments, the CD84 polypeptide comprises or has amino acids 1 to 345, 1 to 50, 50 to 100, 100 to 150, 150 to 200, 226 to 250, or 200 to 345 of SEQ ID NO: 1. In certain embodiments, the CD84 polypeptide comprises or has amino acids 226 to 250 of SEQ ID NO: 1.

SEQ ID NO: 1 is provided below:

[SEQ ID NO: 1]

| | |
|---|---|
| 1 | MAQHHLWILL LCLQTWPEAA GKDSEIFTVN GILGESVTFP |
| | VNIQEPRQVK IIAWTSKTSV |
| 61 | AYVTPGDSET APVVTVTHRN YYERIHALGP NYNLVISDLR |
| | MEDAGDYKAD INTQADPYTT |
| 121 | TKRYNLQIYR RLGKPKITQS LMASVNSTCN VTLTCSVEKE |
| | EKNVTYNWSP LGEEGNVLQI |
| 181 | FQTPEDQELT YTCTAQNPVS NNSDSISARQ LCADIAMGFR |
| | THHTGLLSVL AMFFLLVLIL |
| 241 | SSVFLFRLFK RRQGRIFPEG SCLNTFTKNP YAASKKTIYT |
| | YIMASRNTQP AESRIYDEIL |
| 301 | QSKVLPSKEE PVNTVYSEVQ FADKMGKAST QDSKPPGTSS |
| | YEIVI |

In accordance with the presently disclosed subject matter, a “CD84 nucleic acid molecule” refers to a polynucleotide encoding a CD84 polypeptide. An exemplary nucleotide sequence encoding amino acids 226 to 250 of SEQ ID NO: 1 is set forth in SEQ ID NO: 2, which is provided below.

[SEQ ID NO: 2]
TTGCTGAGCGTGCTGGCTATGTTCTTTCTGCTTGTTCTCATTCTGTCTTCA
GTGTTTTTGTTCCGTTTGTTCAAG

CD166

In certain embodiments, the transmembrane domain of at least one of the two or more CARs comprises a native or modified transmembrane domain of a CD166 polypeptide or a portion thereof. The CD166 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001618.2 (SEQ ID NO: 3), or a fragment thereof, and/or may optionally comprise up to one or up to two or up to three conservative amino acid substitutions. In non-limiting certain embodiments, the CD166 polypeptide comprises or has an amino acid sequence that is a consecutive portion of SEQ ID NO: 3, which is at least about 15, at least about 20, or at least about 30, or at least about 40, or at least about 50, and up to about 583 amino acids in length. Alternatively or additionally, in non-limiting various embodiments, the CD166 polypeptide comprises or has amino acids 1 to 583, 1 to 50, 50 to 100, 100 to 150, 150 to 200, 200 to 300, 300 to 400, 400-500, 500 to 550, 528 to 553, 528 to 549, or 550 to 583 of SEQ ID NO: 3. In certain embodiments, the CD166 polypeptide comprised in the transmembrane domain of at least one of the two or more CARs comprises or has amino acids 528 to 553 of SEQ ID NO: 3. In certain embodiments, the CD166 polypeptide comprised in the transmembrane domain of at least one of the two or more CARs comprises or has amino acids 528 to 549 of SEQ ID NO: 3.

SEQ ID NO: 3 is provided below:

[SEQ ID NO: 3]

| | |
|---|---|
| 1 | MESKGASSCR LLFCLLISAT VFRPGLGWYT VNSAYGDTII |
| | IPCRLDVPQN LMFGKWKYEK |
| 61 | PDGSPVFIAF RSSTKKSVQY DDVPEYKDRL NLSENYTLSI |
| | SNARISDEKR FVCMLVTEDN |
| 121 | VFEAPTIVKV FKQPSKPEIV SKALFLETEQ LKKLGDCISE |
| | DSYPDGNITW YRNGKVLHPL |
| 181 | EGAVVIIFKK EMDPVTQLYT MTSTLEYKTT KADIQMPFTC |
| | SVTYYGPSGQ KTIHSEQAVF |
| 241 | DIYYPTEQVT IQVLPPKNAI KEGDNITLKC LGNGNPPPEE |
| | FLFYLPGQPE GIRSSNTYTL |
| 301 | TDVRRNATGD YKCSLIDKKS MIASTAITVH YLDLSLNPSG |
| | EVTRQIGDAL PVSCTISASR |
| 361 | NATVVWMKDN IRLRSSPSFS SLHYQDAGNY VCETALQEVE |
| | GLKKRESLTL IVEGKPQIKM |
| 421 | TKKTDPSGLS KTIICHVEGF PKPAIQWTIT GSGSVINQTE |
| | ESPYINGRYY SKIIISPEEN |
| 481 | VTLTCTAENQ LERTVNSLNV SAISIPEHDE ADEISDENRE |
| | KVNDQAKLIV GIVVGLLLAA |
| 541 | LVAGVVYWLY MKKSKTASKH VNKDLGNMEE NKKLEENNHK |
| | TEA |

In accordance with the presently disclosed subject matter, a “CD166 nucleic acid molecule” refers to a polynucleotide encoding a CD166 polypeptide. An exemplary nucleotide sequence encoding amino acids 528 to 553 of SEQ ID NO: 3 is set forth in SEQ ID NO: 4, which is provided below.

[SEQ ID NO: 4]
CTAATTGTGGGAATCGTTGTTGGTCTCCTCCTTGCTGCCCTTGTTGCTGGT
GTCGTCTACTGGCTGTACATGAAGAAG

CD8a

In certain embodiments, the transmembrane domain of at least one of the two or more CARs comprises a native or modified transmembrane domain of a CD8a polypeptide or a portion thereof.

The CD8a polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_001139345.1 (SEQ ID NO: 5), or a fragment thereof, and/or may optionally comprise up to one or up to two or up to three conservative amino acid substitutions. In non-limiting certain embodiments, the CD8a polypeptide comprises or has an amino acid sequence that is a consecutive portion of SEQ ID NO: 5, which is at least 20 (e.g., about 25), or at least 30, or at least 40, or at least 50, and up to 235 amino acids in length. Alternatively or additionally, in non-limiting various embodiments, the CD8a polypeptide comprises or has amino acids 1 to 235, 1 to 50, 50 to 100, 100 to 150, 150 to 200, 183 to 207, or 200 to 235 of SEQ ID NO: 5. In certain embodiments, the CD8a polypeptide comprised in the transmembrane domain of at least one of the two or more CARs comprises or has amino acids 183 to 207 of SEQ ID NO: 5.

SEQ ID NO: 5 is provided below:

[SEQ ID NO: 5]

| | |
|---|---|
| 1 | MALPVTALLL PLALLLHAAR PSQFRVSPLD RTWNLGETVE |
| | LKCQVLLSNP TSGCSWLFQP |
| 61 | RGAAASPTFL LYLSQNKPKA AEGLDTQRFS GKRLGDTFVL |
| | TLSDFRRENE GYYFCSALSN |
| 121 | SIMYFSHFVP VFLPAKPTTT PAPRPPTPAP TIASQPLSLR |
| | PEACRPAAGG AVHTRGLDFA |
| 181 | CDIYIWAPLA GTCGVLLLSL VITLYCNHRN RRRVCKCPRP |
| | VVKSGDKPSL SARYV |

In accordance with the presently disclosed subject matter, a “CD8a nucleic acid molecule” refers to a polynucleotide encoding a CD8a polypeptide. An exemplary nucleotide sequence encoding amino acids 183 to 207 of SEQ ID NO: 5 is set forth in SEQ ID NO: 6, which is provided below.

[SEQ ID NO: 6]
atctacatctgggcgcccttggccgggacttgtggggtccttctcctgtca
ctggttatcaccctttactgcaac

CD8b

In certain embodiments, the transmembrane domain of at least one of the two or more CARs comprises a native or modified transmembrane domain of a CD8b polypeptide or a portion thereof. The CD8b polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_742099.1 (SEQ ID NO: 7), or a fragment thereof, and/or may optionally comprise up to one or up to two or up to three conservative amino acid substitutions. In non-limiting certain embodiments, the CD8b polypeptide comprises or has an amino acid sequence that is a consecutive portion of SEQ ID NO: 7, which is at least 20 (e.g., about 25), or at least 30, or at least 40, or at least 50, and up to 220 amino acids in length. Alternatively or additionally, in non-limiting various embodiments, the CD8b polypeptide comprises or has amino acids 1 to 221, 1 to 50, 50 to 100, 100 to 150, 150 to 200, 171 to 195, or 200 to 221 of SEQ ID NO: 7. In certain embodiments, the CD8b polypeptide comprised in the transmembrane domain of at least one of the two or more CARs comprises or has an amino acid sequence of amino acids 171 to 195 of SEQ ID NO: 7.

SEQ ID NO: 7 is provided below:

[SEQ ID NO: 7]

| | |
|---|---|
| 1 | MRPRLWLLLA AQLTVLHGNS VLQQTPAYIK VQTNKMVMLS |
| | CEAKISLSNM RIYWLRQRQA |
| 61 | PSSDSHHEFL ALWDSAKGTI HGEEVEQEKI AVFRDASRFI |
| | LNLTSVKPED SGIYFCMIVG |
| 121 | SPELTFGKGT QLSVVDFLPT TAQPTKKSTL KKRVCRLPRP |
| | ETQKGPLCSP ITLGLLVAGV |
| 181 | LVLLVSLGVA IHLCCRRRRA RLRFMKQLRL HPLEKCSRMD Y |

In accordance with the presently disclosed subject matter, a “CD8b nucleic acid molecule” refers to a polynucleotide encoding a CD8b polypeptide. An exemplary nucleotide sequence encoding amino acids 171 to 195 of SEQ ID NO: 7 is set forth in SEQ ID NO: 8, which is provided below.

[SEQ ID NO: 8]
ATCACCCTTGGCCTGCTGGTGGCTGGCGTCCTGGTTCTGCTGGTTTCCCTG
GGAGTGGCCATCCACCTGTGCTGC

LCOS

In certain embodiments, the transmembrane domain of at least one of the two or more CARs comprises a native or modified transmembrane domain of an ICOS polypeptide or a portion thereof. The ICOS polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_036224.1 (SEQ ID NO: 9), or a fragment thereof, and/or may optionally comprise up to one or up to two or up to three conservative amino acid substitutions. In non-limiting certain embodiments, the ICOS polypeptide comprises or has an amino acid sequence that is a consecutive portion of SEQ ID NO: 9, which is at least 20 (e.g., about 25), or at least 30, or at least 40, or at least 50, and up to 199 amino acids in length. Alternatively or additionally, in non-limiting various embodiments, the ICOS polypeptide comprises or has an amino acid sequence of amino acids 1 to 199, 1 to 50, 50 to 100, 100 to 150, 141 to 165, or 150 to 199 of SEQ ID NO: 9. In certain embodiments, the ICOS polypeptide comprised in the transmembrane domain of at least one of the two or more CARs comprises or has amino acids 141 to 165 of SEQ ID NO: 9.

SEQ ID NO: 9 is provided below:

[SEQ ID NO: 9]

| | |
|---|---|
| 1 | MKSGLWYFFL FCLRIKVLTG EINGSANYEM FIFHNGGVQI |
| | LCKYPDIVQQ FKMQLLKGGQ |
| 61 | ILCDLIKTKG SGNTVSIKSL KFCHSQLSNN SVSFFLYNLD |
| | HSHANYYFCN LSIFDPPPFK |
| 121 | VTLIGGYLHI YESQLCCQLK FWLPIGCAAF VVVCILGCIL |
| | ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide. An exemplary nucleotide sequence encoding amino acids 141 to 165 of SEQ ID NO: 9 is set forth in SEQ ID NO: 10, which is provided below.

[SEQ ID NO: 10]
TTCTGGTTACCCATAGGATGTGCAGCCTTTGTTGTAGTCTGCATTTTGGGA
TGCATACTTATTTGTTGGCTTACA

CTLA-4

In certain embodiments, the transmembrane domain of at least one of the two or more CARs comprises a native or modified transmembrane domain of a CTLA-4 polypeptide or a potion thereof. The CTLA-4 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_005205.2 (SEQ ID NO: 11), or a fragment thereof, and/or may optionally comprise up to one or up to two or up to three conservative amino acid substitutions. In non-limiting certain embodiments, the CTLA-4 polypeptide comprises or has an amino acid sequence that is a consecutive portion of SEQ ID NO: 11, which is at least 20 (e.g., about 25), or at least 30, or at least 40, or at least 50, and up to 223 amino acids in length. Alternatively or additionally, in non-limiting various embodiments, the CTLA-4 polypeptide comprises or has amino acids 1 to 220, 1 to 50, 50 to 100, 100 to 150, 150 to 200, 162 to 186, or 200 to 223 of SEQ ID NO: 11. In certain embodiments, the CTLA-4 polypeptide comprised in the transmembrane domain of at least one of the two or more CARs comprises or has amino acids 162 to 186 of SEQ ID NO: 11.

SEQ ID NO: 11 is provided below:

[SEQ ID NO: 11]

| | |
|---|---|
| 1 | MACLGFQRHK AQLNLATRTW PCTLLFFLLF IPVFCKAMHV |
| | AQPAVVLASS RGIASFVCEY |
| 61 | ASPGKATEVR VTVLRQADSQ VTEVCAATYM MGNELTFLDD |
| | SICTGTSSGN QVNLTIQGLR |
| 121 | AMDTGLYICK VELMYPPPYY LGIGNGTQIY VIDPEPCPDS |
| | DFLLWILAAV SSGLFFYSFL |
| 181 | LTAVSLSKML KKRSPLTTGV YVKMPPTEPE CEKQFQPYFI |
| | PIN |

In accordance with the presently disclosed subject matter, a “CTLA-4 nucleic acid molecule” refers to a polynucleotide encoding a CTLA-4 polypeptide. An exemplary nucleotide sequence encoding amino acids 162 to 186 of SEQ ID NO: 11 is set forth in SEQ ID NO: 12, which is provided below.

[SEQ ID NO: 12]
TTCCTCCTCTGGATCCTTGCAGCAGTTAGTTCGGGGTTGTTTTTTTATAGC
TTTCTCCTCACAGCTGTTTCTTTG

ICAM-1

In certain embodiments, the transmembrane domain of at least one of the two or more CARs comprises a native or modified transmembrane domain of an ICAM-1 polypeptide or a portion thereof. The ICAM-1 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_000192.2 (SEQ ID NO: 13), or a fragment thereof, and/or may optionally comprise up to one or up to two or up to three conservative amino acid substitutions. In non-limiting certain embodiments, the ICAM-1 polypeptide comprises or has an amino acid sequence that is a consecutive portion of SEQ ID NO: 13, which is at least 20, or at least 30, or at least 40, or at least 50, and up to 532 amino acids in length. Alternatively or additionally, in non-limiting various embodiments, the ICAM-1 polypeptide comprises or has amino acids 1 to 532, 1 to 50, 50 to 100, 100 to 150, 150 to 200, 200 to 300, 300 to 400, 400 to 500, 481 to 507, or 500 to 532 of SEQ ID NO: 13. In certain embodiments, the ICAM-1 polypeptide comprised in the transmembrane domain of a presently disclosed CAR comprises or has amino acids 481 to 507 of SEQ ID NO: 13.

SEQ ID NO: 13 is provided below:

[SEQ ID NO: 13]

| | |
|---|---|
| 1 | MAPSSPRPAL PALLVLLGAL FPGPGNAQTS VSPSKVILPR |
| | GGSVLVTCST SCDQPKLLGI |
| 61 | ETPLPKKELL LPGNNRKVYE LSNVQEDSQP MCYSNCPDGQ |
| | STAKTFLTVY WTPERVELAP |
| 121 | LPSWQPVGKN LTLRCQVEGG APRANLTVVL LRGEKELKRE |
| | PAVGEPAEVT TTVLVRRDHH |
| 181 | GANFSCRTEL DLRPQGLELF ENTSAPYQLQ TFVLPATPPQ |
| | LVSPRVLEVD TQGTVVCSLD |
| 241 | GLFPVSEAQV HLALGDQRLN PTVTYGNDSF SAKASVSVTA |
| | EDEGTQRLTC AVILGNQSQE |
| 301 | TLQTVTIYSF PAPNVILTKP EVSEGTEVTV KCEAHPRAKV |
| | TLNGVPAQPL GPRAQLLLKA |
| 361 | TPEDNGRSFS CSATLEVAGQ LIHKNQTREL RVLYGPRLDE |
| | RDCPGNWTWP ENSQQTPMCQ |
| 421 | AWGNPLPELK CLKDGTFPLP IGESVTVTRD LEGTYLCRAR |
| | STQGEVTRKV TVNVLSPRYE |
| 481 | IVIITVVAAA VIMGTAGLST YLYNRQRKIK KYRLQQAQKG |
| | TPMKPNTQAT PP |

In accordance with the presently disclosed subject matter, an “ICAM-1 nucleic acid molecule” refers to a polynucleotide encoding an ICAM-1 polypeptide. An exemplary nucleotide sequence encoding amino acids 481 to 507 of SEQ ID NO: 13 is set forth in SEQ ID NO: 14, which is provided below.

[SEQ ID NO: 14]
ATTGTCATCATCACTGTGGTAGCAGCCGCAGTCATAATGGGCACTGCAGGC
CTCAGCACGTACCTCTATAACCGCCAGCGG

7.2.2.3. Hinge/Spacer Region

In certain non-limiting embodiments, at least one of the two or more CARs comprises a hinge/spacer region that links the extracellular antigen-binding domain to the transmembrane domain. In certain embodiments, the hinge/spacer region is positioned between the extracellular antigen-binding domain and the transmembrane domain. The hinge/spacer region can be flexible enough to allow the antigen binding domain to orient in different directions to facilitate antigen recognition. In certain non-limiting embodiments, the hinge/spacer region of at least one of the two or more CARs comprises a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 polypeptide, a NKGD2 polypeptide, a CD2 polypeptide, a CD7 polypeptide, a LIGHT polypeptide, a NKG2C polypeptide, a B7-H3 polypeptide, a FcεRIγ polypeptide, a TNF receptor polypeptide, an Immunoglobulin-like polypeptide, a cytokine polypeptide, an integrin polypeptide, a signaling lymphocytic activation molecule polypeptide (a SLAM polypeptide), an activating NK cell receptor polypeptide, a BTLA polypeptide, a Toll ligand receptor polypeptide, a CD30 polypeptide, a CDS polypeptide, an ICAM-1 polypeptide, a LFA-1 (CD11a/CD18) polypeptide, a CDS polypeptide, a GITR polypeptide, a BAFFR polypeptide, a HVEM (LIGHTR) polypeptide, a KIRDS2 polypeptide, a SLAMF7 polypeptide, a NKp80 (KLRF1) polypeptide, a NKp44 polypeptide, a NKp30 polypeptide, a NKp46 polypeptide, a CD19 polypeptide, a CD4 polypeptide, a CD8alpha polypeptide, a CD8beta polypeptide, an IL2R beta polypeptide, an IL2R gamma polypeptide, an IL7R alpha polypeptide, an ITGA4 polypeptide, a VLA1 polypeptide, a CD49a polypeptide, an ITGA4 polypeptide, an IA4 polypeptide, a CD49D polypeptide, an ITGA6 polypeptide, a VLA-6 polypeptide, a CD49f polypeptide, an ITGAD polypeptide, a CD11d polypeptide, an ITGAE polypeptide, a CD103 polypeptide, an ITGAL polypeptide, a CD11 a polypeptide, a LFA-1 polypeptide, an ITGAM polypeptide, a CD11b polypeptide, an ITGAX polypeptide, a CD11c polypeptide, an ITGB1 polypeptide, a CD29 polypeptide, an ITGB2 polypeptide, a CD18 polypeptide, a LFA-1 polypeptide, an ITGB7 polypeptide, a NKG2C polypeptide, a TNFR2 polypeptide, a TRANCE/RANKL polypeptide, a DNAM1 (CD226) polypeptide, a SLAMF4 (CD244, 2B4) polypeptide, a CD84 polypeptide, a CD96 (Tactile) polypeptide, a CEACAM1 polypeptide, a CRTAM polypeptide, a Ly9 (CD229) polypeptide, a CD160 (BY55) polypeptide, a PSGL1 polypeptide, a CD100 (SEMA4D) polypeptide, a CD69 polypeptide, a SLAMF6 (NTB-A, Ly108) polypeptide, a SLAM (SLAMF1, CD150, IPO-3) polypeptide, a BLAME (SLAMF8) polypeptide, a SELPLG (CD162) polypeptide, a LTBR polypeptide, a LAT polypeptide, a GADS polypeptide, a SLP-76 polypeptide, a PAG/Cbp polypeptide, a CD19a polypeptide, and a ligand that specifically binds with CD83, a synthetic polypeptide (not based on a protein associated with the immune response), or a combination thereof. The hinge/spacer region can be the hinge region from IgG1, or the CH2CH3 region of immunoglobulin and portions of CD3, a portion of a CD28 polypeptide (e.g., a portion of SEQ ID NO: 90), a portion of a CD8 polypeptide (e.g., a portion of SEQ ID NO: 86, or a portion of SEQ ID NO: 87), a variation of any of the foregoing which is at least about 80%, at least about 85%, at least about 90%, at least about 95%, or at least about 100% homologous or identical thereto, or a synthetic spacer sequence.

CD28

In certain embodiments, the hinge/spacer region of at least one of the two or more CARs comprises a native or modified hinge region of a CD28 polypeptide as described herein. In certain embodiments, the CD28 polypeptide comprised in the hinge/spacer region of at least one of the two or more CARs comprises or has amino acids 114 to 152 of SEQ ID NO: 90. An exemplary nucleotide sequence encoding amino acids 114 to 152 of SEQ ID NO: 90 is set forth in SEQ ID NO: 15, which is provided below.

[SEQ ID NO: 15]
IEVMYPPPYLDNEKSNGTIIHVKGKHLCPSPLFPGPSKP

CD84

In certain embodiments, the hinge/spacer region of at least one of the two or more CARs comprises a native or modified hinge region of a CD84 polypeptide as described herein. In certain embodiments, the CD84 polypeptide comprised in the hinge/spacer region of at least one of the two or more CARs comprises or has an amino acids 187 to 225 of SEQ ID NO: 1. An exemplary nucleotide sequence encoding amino acids 187 to 225 of SEQ ID NO: 1 is set forth in SEQ ID NO: 16, which is provided below.

[SEQ ID NO: 16]
CAAGAGCTGACTTACACGTGTACAGCCCAGAACCCTGTCAGCAACAATTCT
GACTCCATCTCTGCCCGGCAGCTCTGTGCAGACATCGCAATGGGCTTCCGT
ACTCACCACACCGGG

CD166

In certain embodiments, the hinge/spacer region of at least one of the two or more CARs comprises a native or modified hinge region of a CD166 polypeptide as described herein. In certain embodiments, the CD166 polypeptide comprised in the hinge/spacer region of a presently disclosed CAR comprises or has amino acids 489 to 527 of SEQ ID NO:3. An exemplary nucleotide sequence encoding amino acids 489 to 527 of SEQ ID NO: 3 is set forth in SEQ ID NO: 17, which is provided below.

[SEQ ID NO: 17]
ACCAACTGGAGAGAACAGTAAACTCCTTGAATGTCTCTGCTATAAGTATTC
CAGAACACGATGAGGCAGACGAGATAAGTGATGAAAACAGAGAAAAGGTGA
ATGACCAGGCAAAA

In certain embodiments, the CD166 polypeptide comprised in the hinge/spacer region of at least one of the two or more CARs comprises or has amino acids 484 to 527 of SEQ ID NO:3. In certain embodiments, the CD166 polypeptide comprised in the hinge/spacer region of at least one of the two or more CARs comprises or has amino acids 506 to 527 of SEQ ID NO:3. In certain embodiments, the CD166 polypeptide comprised in the hinge/spacer region of at least one of the two or more CARs comprises or has amino acids 517 to 527 of SEQ ID NO:3. In certain embodiments, the CD166 polypeptide comprised in the hinge/spacer region of at least one of the two or more CARs comprises or has the amino acid sequence set forth in SEQ ID NO: 109 or 110.

[SEQ ID NO: 109]
NQLERTVNSLNVPAISIPEHDEADEISDENREKVNDQAK
[SEQ ID NO: 110]
AAANQLERTVNSLNVSAISIPEHDEADEISDENREKVNDQAK

In certain embodiments, the CD166 polypeptide comprised in the hinge/spacer region and the transmembrane domain of at least one of the two or more CARs comprises or has the amino acid sequence set forth in SEQ ID NO: 111, SEQ ID NO: 112, SEQ ID NO: 113, SEQ ID NO: 114, SEQ ID NO: 115, SEQ ID NO: 116, or SEQ ID NO: 117. SEQ ID NOS: 111-117 are provided below.

[SEQ ID NO: 111]
PEHDEADEISDENREKVNDQAKLIVGIVVGLLLAALVAGVVYWLYMKK
[SEQ ID NO: 112]
ENREKVNDQAKLIVGIVVGLLLAALVAGVVYWLYMKK
[SEQ ID NO: 113]
NQLERTVNSLNVPAISIPEHDEADEISDENREKVNDQAKLIVGIVVGLLLA
ALVAGVVYWLYMKK
[SEQ ID NO: 114]
TCTAENQLERTVNSLNVSAISIPEHDEADEISDENREKVNDQAKLIVGIVV
GLLLAALVAGVVYWL
[SEQ ID NO: 115]
PEHDEADEISDENREKVNDQAKLIVGIVVGLLLAALVAGVVYWL
[SEQ ID NO: 116]
NQLERTVNSLNVSAISIPEHDEADEISDENREKVNDQAKLIVGIVVGLLLA
ALVAGVVYWL
[SEQ ID NO: 117]
AAANQLERTVNSLNVSAISIPEHDEADEISDENREKVNDQAKLIVGIVVGL
LLAALVAGVVYWLYMKK

CD8a

In certain embodiments, the hinge/spacer region of at least one of the two or more CARs comprises a native or modified hinge region of a CD8a polypeptide as described herein. In certain embodiments, the CD8a polypeptide comprised in the hinge/spacer region of at least one of the two or more CARs comprises or has amino acids 137 to 182 of SEQ ID NO: 5. An exemplary nucleotide sequence encoding amino acids 137 to 182 of SEQ ID NO: 5 is set forth in SEQ ID NO: 18, which is provided below.

[SEQ ID NO: 18]
cccaccacgacgccagcgccgcgaccaccaacaccggcgcccaccatcgcg
tcgcagcccctgtccctgcgcccagaggcgtgccggccagcggcggggggc
gcagtgcacacgagggggctggacttcgcctgtgat

CD8b

In certain embodiments, the hinge/spacer region of at least one of the two or more CARs comprises a native or modified hinge region of a CD8b polypeptide as described herein. In certain embodiments, the CD8b polypeptide comprised in the hinge/spacer region of at least one of the two or more CARs comprises or has amino acids 132 to 170 of SEQ ID NO: 7. An exemplary nucleotide sequence encoding amino acids 132 to 170 of SEQ ID NO: 7 is set forth in SEQ ID NO: 19, which is provided below.

[SEQ ID NO: 19]
CTGAGTGTGGTTGATTTCCTTCCCACCACTGCCCAGCCCACCAAGAAGTCC
ACCCTCAAGAAGAGAGTGTGCCGGTTACCCAGGCCAGAGACCCAGAAGGGC
CCACTTTGTAGCCCC

ICOS

In certain embodiments, the hinge/spacer region of at least one of the two or more CARs comprises a native or modified hinge region of an ICOS polypeptide as described herein. In certain embodiments, the ICOS polypeptide comprised in the hinge/spacer region of at least one of the two or more CARs comprises or has amino acids 102 to 140 of SEQ ID NO: 9. An exemplary nucleotide sequence encoding amino acids 102 to 140 of SEQ ID NO: 9 is set forth in SEQ ID NO: 20, which is provided below.

[SEQ ID NO: 20]
tctcatgccaactattacttctgcaacctatcaatttttgatcctcctcct
tttaaagtaactcttacaggaggatatttgcatatttatgaatcacaactt
tgttgccagctgaag

CTLA-4

In certain embodiments, the hinge/spacer region of at least one of the two or more CARs comprises a native or modified hinge region of a CTLA-4 polypeptide as described herein. In certain embodiments, the CTLA-4 polypeptide comprised in the hinge/spacer region of at least one of the two or more CARs comprises or has amino acids 123 to 161 of SEQ ID NO: 11. An exemplary nucleotide sequence encoding amino acids 123 to 161 of SEQ ID NO: 11 is set forth in SEQ ID NO: 21, which is provided below.

[SEQ ID NO: 21]
GACACGGGACTCTACATCTGCAAGGTGGAGCTCATGTACCCACCGCCATAC
TACCTGGGCATAGGCAACGGAACCCAGATTTATGTAATTGATCCAGAACCG
TGCCCAGATTCTGAC

ICAM-1

In certain embodiments, the hinge/spacer region of at least one of the two or more CARs comprises a native or modified hinge region of a ICAM-1 polypeptide as described herein. In certain embodiments, the ICAM-1 polypeptide comprised in the hinge/spacer region of at least one of the two or more CARs comprises or has amino acids 442 to 480 of SEQ ID NO: 13. An exemplary nucleotide sequence encoding amino acids 442 to 480 of SEQ ID NO: 13 is set forth in SEQ ID NO: 22, which is provided below.

[SEQ ID NO: 22]
GGGGAATCAGTGACTGTCACTCGAGATCTTGAGGGCACCTACCTCTGTCGG
GCCAGGAGCACTCAAGGGGAGGTCACCCGCAAGGTGACCGTGAATGTGCTC
TCCCCCCGGTATGAG

In certain embodiments, the transmembrane domain and the hinge/spacer region are derived from the same molecule. In certain embodiments, the transmembrane domain and the hinge/spacer region are derived from different molecules. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD84 polypeptide and the transmembrane domain of the CAR comprises a CD84 polypeptide. In certain embodiments, the hinge/spacer region of the CAR comprises a CD166 polypeptide and the transmembrane domain of the CAR comprises a CD166 polypeptide. In certain embodiments, the hinge/spacer region of the CAR comprises a CD8a polypeptide and the transmembrane domain of the CAR comprises a CD8a polypeptide. In certain embodiments, the hinge/spacer region of the CAR comprises a CD8b polypeptide and the transmembrane domain of the CAR comprises a CD8b polypeptide. In certain embodiments, the hinge/spacer region of the CAR comprises a CD28 polypeptide and the transmembrane domain of the CAR comprises an ICOS polypeptide.

7.2.2.4. Intracellular Signaling Domain of a CAR

In certain non-limiting embodiments, the intracellular signaling domain of at least one of the two or more CARs comprises a CD3ζ polypeptide, which can activate or stimulate a cell (e.g., a cell of the lymphoid lineage, e.g., a T cell). Wild type (“native”) CD3 comprises three immunoreceptor tyrosine-based activation motifs (“ITAMs”) (e.g., ITAM1, ITAM2 and ITAM3), three basic-rich stretch (BRS) regions (BRS1, BRS2 and BRS3), and transmits an activation signal to the cell (e.g., a cell of the lymphoid lineage, e.g., a T cell) after antigen is bound. The intracellular signaling domain of the native CD3-chain is the primary transmitter of signals from endogenous TCRs.

In certain non-limiting embodiments, the intracellular signaling domain of at least one of the two or more CARs comprises a native CD3ζ polypeptide.

In certain non-limiting embodiments, the intracellular signaling domain of at least one of the two or more CARs comprises a modified CD3ζ polypeptide. In certain embodiments, the CD3ζ polypeptide comprises or has an amino acid sequence that is at least about 80%, at least about 85%, at least about 90%, at least about 95%, at least about 96%, at least about 97%, at least about 98%, or at least about 99%, at least about 100% homologous or identical to the sequence having a NCBI Reference No: NP_932170 (SEQ ID NO: 94), or a fragment thereof. In certain non-limiting embodiments, the CD3ζ polypeptide comprises or has an amino acid sequence that is a consecutive portion of SEQ ID NO: 94, which is at least 20, or at least 30, or at least 40, or at least 50, or at least 100, or at least 110, or at least 113, and up to 163 amino acids in length. Alternatively or additionally, in non-limiting various embodiments, the CD3ζ polypeptide comprises or has amino acids 1 to 50, 50 to 100, 100 to 150, 50 to 164, 55 to 164, or 150 to 164 of SEQ ID NO: 94. In certain embodiments, the CD3ζ polypeptide comprises or has amino acids 52 to 164 of SEQ ID NO: 94.

SEQ ID NO: 94 is provided below:

[SEQ ID NO: 94]

| | |
|---|---|
| 1 | MKWKALFTAA ILQAQLPITE AQSFGLLDPK LCYLLDGILF |
| | IYGVILTALF LRVKFSRSAD |
| 61 | APAYQQGQNQ LYNELNLGRR EEYDVLDKRR GRDPEMGGKP |
| | QRRKNPQEGL YNELQKDKMA |
| 121 | EAYSEIGMKG ERRRGKGHDG LYQGLSTATK DTYDALHMQA |
| | LPPR |

In certain embodiments, the intracellular signaling domain of at least one of the two or more CARs comprises a CD3ζ polypeptide that comprises or has an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 95 or a fragment thereof, and/or may optionally comprise up to one or up to two or up to three conservative amino acid substitutions. SEQ ID NO: 95 is provided below:

[SEQ ID NO: 95]
RVKFSRSADA PAYQQGQNQL YNELNLGRRE EYDVLDKRRG
RDPEMGGKPR RKNPQEGLYN ELQKDKMAEA YSEIGMKGER
RRGKGHDGLY QGLSTATKDT YDALHMQALP PR.

An exemplary nucleotide sequence encoding the amino acid sequence of SEQ ID NO: 95 is set forth in SEQ ID NO: 96, which is provided below.

[SEQ ID NO: 96]
AGAGTGAAGTTCAGCAGGAGCGCAGACGCCCCCGCGTACCAGCAGGGCCAG
AACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGG
AAGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGGG
CACGATGGCCTTTACCAGGGTCTCAGTACAGCCACCAAGGACACCTACGAC
GCCCTTCACATGCAGGCCCTGCCCCCTCGC

An exemplary nucleotide sequence encoding the amino acid sequence of SEQ ID NO: 95 is set forth in SEQ ID NO: 59, which is provided below.

[SEQ ID NO: 59]
AGAGTGAAGTTTAGCCGGAGTGCCGATGCTCCAGCCTATCAGCAAGGACAA
AACCAACTTTACAACGAACTTAATCTCGGTAGGCGAGAGGAATACGATGTG
CTTGATAAACGCCGAGGTCGAGATCCCGAAATGGGCGGGAAACCGCGACGC
AAGAATCCTCAAGAAGGACTCTATAATGAGTTGCAGAAGGACAAGATGGCT
GAGGCATATAGTGAGATCGGTATGAAGGGAGAACGGAGAAGGGGGAAAGGG
CATGATGGATTGTACCAAGGACTTAGCACAGCTACTAAAGATACATATGAC
GCCCTGCACATGCAAGCATTGCCTCCACGC

In certain embodiments, the intracellular signaling domain of at least one of the two or more CARs comprises a modified CD3ζ polypeptide. In certain embodiments, the intracellular signaling domain of at least one of the two or more CARs comprises a modified human CD3ζ polypeptide. In certain embodiments, the modified CD3ζ polypeptide comprises or has an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 135 or a fragment thereof, and/or may optionally comprise up to one or up to two or up to three conservative amino acid substitutions. SEQ ID NO: 135 is provided below:

[SEQ ID NO: 135]
RVKFSRSADAPAYQQGQNQLYNELNLGRREEYDVLDKRRGRDPEMGGKPRR
KNPQEGLFNELQKDKMAEAFSEIGMKGERRRGKGHDGLFQGLSTATKDTFD
ALHMQALPPR

An exemplary nucleic acid sequence encoding the amino acid sequence of SEQ ID NO: 135 is set forth in SEQ ID NO: 136, which is provided below.

[SEQ ID NO: 136]
AGAGTGAAGTTCAGCAGGAGCGCAGACGCCCCCGCGTACCAGCAGGGCCAG
AACCAGCTCTATAACGAGCTCAATCTAGGACGAAGAGAGGAGTACGATGTT
TTGGACAAGAGACGTGGCCGGGACCCTGAGATGGGGGGAAAGCCGAGAAGG
AAGAACCCTCAGGAAGGCCTGTTCAATGAACTGCAGAAAGATAAGATGGCG
GAGGCCTTCAGTGAGATTGGGATGAAAGGCGAGCGCCGGAGGGGCAAGGGG
CACGATGGCCTTTTCCAGGGTCTCAGTACAGCCACCAAGGACACCTTCGAC
GCCCTTCACATGCAGGCCCTGCCCCCTCGC

In certain embodiments, the intracellular signaling domain of at least one of the two or more CARs comprises a modified CD3ζ polypeptide. In certain embodiments, the intracellular signaling domain of at least one of the two or more CARs comprises a modified human CD3ζ polypeptide. In certain embodiments, the modified CD3ζ polypeptide comprises or has an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 137 or a fragment thereof, and/or may optionally comprise up to one or up to two or up to three conservative amino acid substitutions. SEQ ID NO: 137 is provided below:

[SEQ ID NO: 137]
RVKFSRSADAPAYQQGQNQLYNELNLGRREEYDVLDKRRGRDP

An exemplary nucleic acid sequence encoding the amino acid sequence of SEQ ID NO: 137 is set forth in SEQ ID NO: 138, which is provided below.

[SEQ ID NO: 138]
AGAGTGAAGTTCAGCAGGAGCGCAGACGCCCCCGCGTACCAGCAGGGCCAG
AACCAGCTCTATAACGAGCTCAATCTAGGACGAAGAGAGGAGTACGATGTT
TTGGACAAGAGACGTGGCCGGGACCCT

In certain embodiments, the intracellular signaling domain of at least one of the two or more CARs comprises two or more copies of a CD3ζ polypeptide. In certain embodiments, the intracellular signaling domain of at least one of the two or more CARs comprises two copies of a CD3ζ polypeptide. In certain embodiments, the intracellular signaling domain of at least one of the two or more CARs comprises three copies of a CD3ζ polypeptide. In certain embodiments, the intracellular signaling domain of at least one of the two or more CARs comprises two copies of a CD3ζ polypeptide having the amino acid sequence set forth in SEQ ID NO: 95. In certain embodiments, the intracellular signaling domain of at least one of the two or more CARs comprises two copies of a CD3ζ polypeptide having amino acids 52 to 164 of SEQ ID NO: 94. CARs comprising multiple copies of CD3ζ polypeptide have been reported to improve anti-tumor efficiency of CAR-T cells, especially for low-antigen density cells. See e.g., Majzner et al., Blood (2018);132 (Supplement 1):963, which is incorporated by reference in its entirety.

In certain embodiments, the two or more copies of the CD3ζ polypeptide are connected directly, e.g., without a linker. In certain embodiments, the two or more copies of the CD3ζ polypeptide are connected with a linker. In certain embodiments, the linker has the amino acid sequence set forth in SEQ ID NO: 60. An exemplary nucleic acid sequence encoding the amino acid sequence of SEQ ID NO: 60 is set forth in SEQ ID NO: 61. SEQ ID NOS: 60 and 61 are provided below:

(SEQ ID NO: 60)
GGGGS
[SEQ ID NO: 61]
GGTGGGGGTGGTTCC

In certain embodiments, the linker has the amino acid sequence set forth in SEQ ID NO: 62. An exemplary nucleic acid sequence encoding the amino acid sequence of SEQ ID NO: 62 is set forth in SEQ ID NO: 63. SEQ ID NOS: 62 and 63 are provided below:

(SEQ ID NO: 62)
GGGGSGGGGS
[SEQ ID NO: 63]
GGTGGGGGTGGTTCCGGGGGAGGCGGCTCA

In certain embodiments, the linker has the amino acid sequence set forth in SEQ ID NO: 66. An exemplary nucleic acid sequence encoding the amino acid sequence of SEQ ID NO: 66 is set forth in SEQ ID NO: 64, which is provided below:

[SEQ ID NO: 64]
GGTGGGGGTGGTTCCGGGGGAGGCGGCTCAGGAGGTGGAGGTTCT

Immunoreceptor Tyrosine-Based Activation Motifs (ITAMs)

In certain non-limiting embodiments, the intracellular signaling domain of at least one of the two or more CARs comprises a modified CD3ζ polypeptide comprising one, two or three ITAMs. In certain embodiments, the modified CD3ζ polypeptide comprises a native ITAM1 comprising the amino acid sequence set forth in SEQ ID NO: 23.

[SEQ ID NO: 23]
QNQLYNELNLGRREEYDVLDKR

An exemplary nucleotide sequence encoding the amino acid sequence of SEQ ID NO: 23 is set forth in SEQ ID NO: 24, which is provided below.

[SEQ ID NO: 24]
CAGAACCAGCTCTATAACGAGCTCAATCTAGGACGAAGAGAGGAGTACGAT
GTTTTGGACAAGAGA

In certain embodiments, the modified CD3ζ polypeptide comprises an ITAM1 variant comprising one or more loss-of-function mutations. In certain embodiments, the modified CD3ζ polypeptide consists of an ITAM1 variant comprising two loss-of-function mutations. In certain embodiments, the loss of function mutation comprises a mutation of a tyrosine residue in ITAM1. In certain embodiments, the ITAM1 variant consisting of two loss-of-function mutations comprises the amino acid sequence set forth in SEQ ID NO: 25, which is provided below.

[SEQ ID NO: 25]
QNQLFNELNLGRREEFDVLDKR

An exemplary nucleotide sequence encoding the amino acid sequence of SEQ ID NO: 25 is set forth in SEQ ID NO: 26, which is provided below.

[SEQ ID NO: 26]
CAGAACCAGCTCTTTAACGAGCTCAATCTAGGAGAAGAGAGGAGTTCGATG
TTTTGGACAAGAGA

In certain embodiments, the modified CD3ζ polypeptide comprises a native ITAM2 comprising the amino acid sequence set forth in SEQ ID NO: 27, which is provided below.

[SEQ ID NO: 27]
QEGLYNELQKDKMAEAYSEIGMK

An exemplary nucleotide sequence encoding the amino acid sequence of SEQ ID NO: 27 is set forth in SEQ ID NO: 28, which is provided below.

[SEQ ID NO: 28]
CAGGAAGGCCTGTACAATGAACTGCAGAAAGATAAGATGGCGGAGGCCTAC
AGTGAGATTGGGATGAAA

In certain embodiments, the modified CD3ζ polypeptide comprises an ITAM2 variant comprising one or more loss-of-function mutations. In certain embodiments, the modified CD3ζ polypeptide consists of an ITAM2 variant comprising two loss-of-function mutations. In certain embodiments, the loss of function mutation comprises a mutation of a tyrosine residue in ITAM2. In certain embodiments, the ITAM2 variant consisting of two loss-of-function mutations comprises the amino acid sequence set forth in SEQ ID NO: 29, which is provided below.

[SEQ ID NO: 29]
QEGLFNELQKDKMAEAFSEIGMK

An exemplary nucleotide sequence encoding the amino acid sequence of SEQ ID NO: 29 is set forth in SEQ ID NO: 30, which is provided below.

[SEQ ID NO: 30]
CAGGAAGGCCTGTTCAATGAACTGCAGAAAGATAAGATGGCGGAGGCCTTC
AGTGAGATTGGGATGAAA

In certain embodiments, the modified CD3ζ polypeptide comprises a native ITAM3 comprising the amino acid sequence set forth in SEQ ID NO: 31, which is provided below.

[SEQ ID NO: 31]
HDGLYQGLSTATKDTYDALHMQ

An exemplary nucleotide sequence encoding the amino acid sequence of SEQ ID NO: 131 is set forth in SEQ ID NO: 32, which is provided below.

[SEQ ID NO: 32]
cacgatggcctttaccagggtctcagtacagccaccaaggacacctacgac
gcccttcacatgcag

In certain embodiments, the modified CD3ζ polypeptide comprises an ITAM3 variant comprising one or more loss-of-function mutations. In certain embodiments, the modified CD3ζ polypeptide consists of an ITAM3 variant comprising two loss-of-function mutations. In certain embodiments, the loss of function mutation comprises a mutation of a tyrosine residue in ITAM3. In certain embodiments, the ITAM3 variant consisting of two loss-of-function mutations comprises the amino acid sequence set forth in SEQ ID NO: 33, which is provided below.

[SEQ ID NO: 33]
HDGLFQGLSTATKDTFDALHMQ

An exemplary nucleotide sequence encoding the amino acid sequence of SEQ ID NO: 33 is set forth in SEQ ID NO: 34, which is provided below.

[SEQ ID NO: 34]
CACGATGGCCTTTTCCAGGGGCTCAGTACAGCCACCAAGGACACCTTCGAC
GCCCTTCACATGCAG

In certain embodiments, the intracellular signaling domain of at least one of the two or more CARs comprises a modified CD3ζ polypeptide comprising or consisting essentially of or consisting of an ITAM1 variant comprising one or more loss-of-function mutations, an ITAM2 variant comprising one or more loss-of-function mutations, and an ITAM3 variant comprising one or more loss-of-function mutations, or a combination thereof. In certain embodiments, the intracellular signaling domain of modified CD3ζ polypeptide comprises a modified CD3ζ polypeptide comprising an ITAM2 variant comprising one or more (e.g., two) loss-of-function mutations and an ITAM3 variant comprising one or more (e.g., two) loss-of-function mutations.

In certain embodiments, the intracellular signaling domain of at least one of the two or more CARs comprises or consists essentially of or consists of a modified CD3ζ polypeptide comprising or consisting essentially of or consisting of a native ITAM1, an ITAM2 variant comprising two loss-of-function mutations and an ITAM3 variant comprising two loss-of-function mutations. In certain embodiments, the intracellular signaling domain of at least one of the two or more CARs comprises a modified CD3ζ polypeptide comprising a native ITAM1 having the amino acid sequence set forth in SEQ ID NO: 23, an ITAM2 variant having the amino acid sequence set forth in SEQ ID NO: 29, and an ITAM3 variant having the amino acid sequence set forth in SEQ ID NO: 33 (e.g., a construct designated as “1XX”).

In certain embodiments, the intracellular signaling domain of at least one of the two or more CARs comprises a modified CD3ζ polypeptide comprising or consisting essentially of or consisting of an ITAM1 variant comprising one or more (e.g., two) loss-of-function mutations and an ITAM3 variant comprising one or more (e.g., two) loss-of-function mutations. In certain embodiments, the intracellular signaling domain of at least one of the two or more CARs comprises a modified CD3ζ polypeptide comprising or consisting essentially of or consisting of an ITAM1 variant comprising two loss-of-function mutations, a native ITAM2, and an ITAM3 variant comprising two loss-of-function mutations. In certain embodiments, the intracellular signaling domain of at least one of the two or more CARs comprises a modified CD3ζ polypeptide comprising or consisting essentially of or consisting of an ITAM1 variant having the amino acid sequence set forth in SEQ ID NO: 25, a native ITAM2 having the amino acid sequence set forth in SEQ ID NO: 27 and an ITAM3 variant having the amino acid sequence set forth in SEQ ID NO: 33 (e.g., a construct designated as “X2X”).

In certain embodiments, the intracellular signaling domain of at least one of the two or more CARs comprises a modified CD3ζ polypeptide comprising or consisting essentially of or consisting of an ITAM1 variant comprising one or more (e.g., two) loss-of-function mutations and an ITAM2 variant comprising one or more (e.g., two) loss-of-function mutations. In certain embodiments, the intracellular signaling domain of at least one of the two or more CARs comprises a modified CD3ζ polypeptide comprising or consisting essentially of or consisting of an ITAM1 variant comprising two loss-of-function mutations, an ITAM2 variant comprising two loss-of-function mutations, and a native ITAM3. In certain embodiments, the intracellular signaling domain of at least one of the two or more CARs comprises a modified CD3ζ polypeptide comprising or consisting essentially of or consisting of an ITAM1 variant having the amino acid sequence set forth in SEQ ID NO: 25, an ITAM2 variant having the amino acid sequence set forth in SEQ ID NO: 29 and a native ITAM3 having the amino acid sequence set forth in SEQ ID NO: 31 (e.g., a construct designated as “XX3”).

In certain embodiments, the intracellular signaling domain of at least one of the two or more CARs comprises a modified CD3ζ polypeptide comprising an ITAM1 variant comprising one or more (e.g., two) loss-of-function mutations. In certain embodiments, the intracellular signaling domain of at least one of the two or more CARs comprises a modified CD3ζ polypeptide comprising an ITAM1 variant comprising or consisting essentially of or consisting of two loss-of-function mutations, a native ITAM2, and a native ITAM3. In certain embodiments, the intracellular signaling domain of at least one of the two or more CARs comprises a modified CD3ζ polypeptide comprising or consisting essentially of or consisting of an ITAM1 variant having the amino acid sequence set forth in SEQ ID NO: 25, a native ITAM2 having the amino acid sequence set forth in SEQ ID NO: 27 and a native ITAM3 having the amino acid sequence set forth in SEQ ID NO: 31 (e.g., a construct designated as “X23”).

In certain embodiments, the intracellular signaling domain of at least one of the two or more CARs comprises a modified CD3ζ polypeptide comprising a native ITAM1, a native ITAM2, and an ITAM3 variant comprising one or more (e.g., two) loss-of-function mutations. In certain embodiments, the intracellular signaling domain of at least one of the two or more CARs comprises a modified CD3ζ polypeptide comprising or consisting essentially of or consisting of a native ITAM1, a native ITAM2, and an ITAM1 variant comprising two loss-of-function mutations. In certain embodiments, the intracellular signaling domain of at least one of the two or more CARs comprises a modified CD3ζ polypeptide comprising or consisting essentially of or consisting of a native ITAM1 having the amino acid sequence set forth in SEQ ID NO: 23, a native ITAM2 having the amino acid sequence set forth in SEQ ID NO: 27 and an ITAM3 variant having the amino acid sequence set forth in SEQ ID NO: 33 (e.g., a construct designated as “12X”).

In certain embodiments, the intracellular signaling domain of at least one of the two or more CARs comprises a modified CD3ζ polypeptide comprising a native ITAM1, an ITAM2 variant comprising one or more (e.g., two) loss-of-function mutations, and a native ITAM3. In certain embodiments, the intracellular signaling domain of at least one of the two or more CARs comprises a modified CD3ζ polypeptide comprising or consisting essentially of or consisting of a native ITAM1, an ITAM2 variant comprising two loss-of-function mutations, and a native ITAM3. In certain embodiments, the intracellular signaling domain of at least one of the two or more CARs comprises a modified CD3ζ polypeptide comprising or consisting essentially of or consisting of a native ITAM1 having the amino acid sequence set forth in SEQ ID NO: 23, an ITAM2 variant having the amino acid sequence set forth in SEQ ID NO: 29 and a native ITAM3 having the amino acid sequence set forth in SEQ ID NO: 31 (e.g., a construct designated as “1X3”).

In certain embodiments, the intracellular signaling domain of at least one of the two or more CARs comprises a modified CD3ζ polypeptide comprising a deletion of one or two ITAMs. In certain embodiments, the modified CD3ζ polypeptide comprises a deletion of ITAM1 and ITAM2, e.g., the modified CD3ζ polypeptide comprises a native ITAM3 or a ITAM3 variant, and does not comprise an ITAM1 or an ITAM2. In certain embodiments, the modified CD3ζ polypeptide comprises a native ITAM3 having the amino acid sequence set forth in SEQ ID NO: 31, and does not comprise an ITAM1 (native or modified), or an ITAM2 (native or modified) (e.g., a construct designated as “D12”). In certain embodiments, the modified CD3ζ polypeptide comprises an ITAM3 variant having the amino acid sequence set forth in SEQ ID NO: 33, and does not comprise an ITAM1 (native or modified), or an ITAM2 (native or modified).

In certain embodiments, the modified CD3ζ polypeptide comprises a deletion of ITAM2 and ITAM3, e.g., the modified CD3ζ polypeptide comprises a native ITAM1 or a ITAM1 variant, and does not comprise an ITAM2 or an ITAM3. In certain embodiments, the modified CD3ζ polypeptide comprises a native ITAM1 having the amino acid sequence set forth in SEQ ID NO: 23, and does not comprise an ITAM2 (native or modified), or an ITAM3 (native or modified) (e.g., a construct designated as “D23”). In certain embodiments, the modified CD3ζ polypeptide comprises an ITAM1 variant having the amino acid sequence set forth in SEQ ID NO: 25, and does not comprise an ITAM2 (native or modified), or an ITAM3 (native or modified).

In certain embodiments, the modified CD3ζ polypeptide comprises a deletion of ITAM1 and ITAM3, e.g., the modified CD3ζ polypeptide comprises a native ITAM2 or a ITAM2 variant, and does not comprise an ITAM1 or an ITAM3. In certain embodiments, the modified CD3ζ polypeptide comprises a native ITAM2 having the amino acid sequence set forth in SEQ ID NO: 27, and does not comprise an ITAM1 (native or modified), or an ITAM3 (native or modified) (e.g., a construct designated as “D13”). In certain embodiments, the modified CD3ζ polypeptide comprises an ITAM2 variant having the amino acid sequence set forth in SEQ ID NO: 29, and does not comprise an ITAM1 (native or modified), or an ITAM3 (native or modified).

In certain embodiments, the modified CD3ζ polypeptide comprises a deletion of ITAM1, e.g., the modified CD3ζ polypeptide comprises a native ITAM2 or an ITAM2 variant, and a native ITAM3 or an ITAM3 variant, and does not comprise an ITAM1 (native or modified).

In certain embodiments, the modified CD3ζ polypeptide comprises a deletion of ITAM2, e.g., the modified CD3ζ polypeptide comprises a native ITAM1 or an ITAM1 variant, and a native ITAM3 or an ITAM3 variant, and does not comprise an ITAM2 (native or modified).

In certain embodiments, the modified CD3ζ polypeptide comprises a deletion of ITAM3, e.g., the modified CD3ζ polypeptide comprises a native ITAM1 or an ITAM1 variant, and a native ITAM2 or an ITAM2 variant, and does not comprise an ITAM3 (native or modified). Basic-rich stretch (BRS) region

In certain non-limiting embodiments, the intracellular signaling domain of at least one of the two or more CARs comprises a modified CD3ζ polypeptide comprising one, two or three BRS regions (i.e., BRS1, BRS2, and BRS3). The BRS region can be a native BRS or a modified BRS (e.g., a BRS variant). In certain embodiments, the modified CD3ζ polypeptide comprises a native BRS1 region comprising or having the amino acid sequence set forth in SEQ ID NO: 35, which is provided below.

[SEQ ID NO: 35]
KRRGR

An exemplary nucleotide sequence encoding the amino acid sequence of SEQ ID NO: 35 is set forth in SEQ ID NO: 36, which is provided below.

[SEQ ID NO: 36]
AAGAGACGTGGCCGG

In certain embodiments, the modified CD3ζ polypeptide comprises a BRS1 variant comprising one or more loss-of-function mutations.

In certain embodiments, the modified CD3ζ polypeptide comprises a native BRS2 comprising or having the amino acid sequence set forth in SEQ ID NO: 37.

[SEQ ID NO: 37]
KPRRK

An exemplary nucleotide sequence encoding the amino acid sequence of SEQ ID NO: 37 is set forth in SEQ ID NO: 38, which is provided below.

[SEQ ID NO: 38]
AAGCCGAGAAGGAAG

In certain embodiments, the modified CD3ζ polypeptide comprises a BRS2 variant comprising one or more loss-of-function mutations.

In certain embodiments, the modified CD3ζ polypeptide comprises a native BRS3 comprising or having the amino acid sequence set forth in SEQ ID NO: 39.

[SEQ ID NO: 39]
KGERRRGK

An exemplary nucleotide sequence encoding the amino acid sequence of SEQ ID NO: 39 is set forth in SEQ ID NO: 40, which is provided below.

[SEQ ID NO: 40]
AAAGGCGAGCGCCGGAGGGGCAAG

In certain embodiments, the modified CD3ζ polypeptide comprises a BRS3 variant comprising one or more loss-of-function mutations.

In certain embodiments, the intracellular signaling domain of at least one of the two or more CARs comprises a modified CD3ζ polypeptide comprising all three BRS regions, i.e., a BRS1 region, a BRS2 region, and a BRS3 region. In certain embodiments, the intracellular signaling domain of at least one of the two or more CARs comprises a modified CD3ζ polypeptide comprising a native BRS1, a native BRS2, and a native BRS3. In certain embodiments, the intracellular signaling domain of at least one of the two or more CARs comprises a modified CD3ζ polypeptide comprising a native BRS1 having the amino acid sequence set forth in SEQ ID NO: 35, a native BRS2 having the amino acid sequence set forth in SEQ ID NO: 37, and a native BRS3 having the amino acid sequence set forth in SEQ ID NO: 39, e.g., the modified CD3ζ polypeptide comprised in construct 1XX.

In certain embodiments, the intracellular signaling domain of at least one of the two or more CARs comprises a modified CD3ζ polypeptide comprising one or two but not all three BRS regions. In certain embodiments, the modified CD3ζ polypeptide comprises a BRS1 region and a BRS2 region, and does not comprise a BRS3 region. In certain embodiments, the modified CD3ζ polypeptide comprises a BRS1 region and a BRS3 region, and does not comprise a BRS2 region. In certain embodiments, the modified CD3ζ polypeptide comprises a BRS2 region and a BRS3 region, and does not comprise a BRS1 region.

In certain embodiments, the modified CD3ζ polypeptide comprises a BRS1 region, and does not comprise a BRS2 region or a BRS3 region. In certain embodiments, the modified CD3ζ polypeptide comprises a native BRS1 having the amino acid sequence set forth in SEQ ID NO: 35, and does not comprise a BRS2 region or a BRS3 region, e.g., the modified CD3ζ polypeptide comprised in construct D23. In certain embodiments, the modified CD3ζ polypeptide comprises a BRS2 region, and does not comprise a BRS1 region or BRS3 region. In certain embodiments, the modified CD3ζ polypeptide comprises a BRS3 region, and does not comprise a BRS1 region or a BRS2 region.

In certain embodiments, the modified CD3ζ polypeptide does not comprise a BRS region (native or modified BRS1, BRS2 or BRS3), e.g., all three BRSs are deleted, e.g., the modified CD3t polypeptide comprised in construct D12.

In certain non-limiting embodiments, at least one of the two or more CARs comprises an extracellular antigen-binding domain, a transmembrane domain, and an intracellular signaling domain comprising a modified CD3ζ polypeptide, wherein the modified CD3ζ polypeptide lacks all or part of ITAMs, wherein the ITAMs are ITAM1, ITAM2, and ITAM3.

In certain embodiments, the modified CD3ζ polypeptide lacks ITAM2 or a portion thereof. In certain embodiments, the modified CD3ζ polypeptide further lacks ITAM3 or a portion thereof. In certain embodiments, the modified CD3ζ polypeptide further lacks ITAM1 or a portion thereof.

In certain embodiments, the modified CD3ζ polypeptide lacks ITAM1 or a portion thereof. In certain embodiments, the modified CD3ζ polypeptide further lacks ITAM3 or a portion thereof.

In certain embodiments, the modified CD3ζ polypeptide lacks ITAM3 or a portion thereof.

In certain embodiments, the modified CD3ζ polypeptide lacks all or part of basic-rich stretch (BRS) regions, wherein the BRS regions are BRS1, BRS2, and BRS3.

In certain embodiments, the modified CD3ζ polypeptide lacks BRS2 or a portion thereof In certain embodiments, the modified CD3ζ polypeptide further lacks BRS3 or a portion thereof. In certain embodiments, the modified CD3ζ polypeptide further lacks BRS1 or a portion thereof.

In certain embodiments, the modified CD3ζ polypeptide lacks BRS1 or a portion thereof In certain embodiments, the modified CD3ζ polypeptide further lacks BRS3 or a portion thereof.

In certain embodiments, the modified CD3ζ polypeptide lacks BRS3 or a portion thereof.

In certain embodiments, the modified CD3ζ polypeptide lacks BRS1 or portion thereof, BRS2 or portion thereof, and BRS3 or a portion thereof.

In certain embodiments, the modified CD3ζ polypeptide lacks ITAM2, ITAM3, BRS2, and BRS3. In certain embodiments, at least one of the two or more CARs comprises the amino acid sequence set forth in SEQ ID NO: 45 or SEQ ID NO: 47. In certain embodiments, at least one of the two or more CARs comprises an extracellular antigen-binding domain, a transmembrane domain, and an intracellular signaling domain comprising a modified CD3ζ polypeptide, wherein the modified CD3ζ polypeptide lacks all or part of BRSs, wherein the BRS regions are BRS1, BRS2, and BRS3. In certain embodiments, at least one of the two or more CARs comprises an extracellular antigen-binding domain, a transmembrane domain, and an intracellular signaling domain comprising a modified CD3ζ polypeptide, wherein the modified CD3ζ polypeptide comprises a BRS variant selected from a BRS1 variant, a BRS2 variant, and a BRS3 variant, wherein the BRS variant comprises one or more loss-of-function mutations.

Co-stimulatory Signaling Region

In certain non-limiting embodiments, the intracellular signaling domain of at least one or each of the two or more CARs CAR further comprises at least a co-stimulatory signaling region. In certain embodiments, the co-stimulatory signaling region comprises at least one co-stimulatory molecule or a portion thereof.

As used herein, “co-stimulatory molecules” refer to cell surface molecules other than antigen receptors or their ligands that are required for an efficient response of lymphocytes to antigen. Co-stimulatory molecules can provide optimal lymphocyte activation. The at least one co-stimulatory signaling region can include a CD28 polypeptide, a 4-1BB polypeptide, an OX40 polypeptide, an ICOS polypeptide, a DAP-10 polypeptide, a CD27 peptide, a CD40/My88 polypeptide, a NKGD2 polypeptide a CD2 polypeptide, a CD7 polypeptide, a LIGHT polypeptide, a NKG2C polypeptide, a B7-H3 polypeptide, a FcεRIγ polypeptide, a TNF receptor polypeptide, an Immunoglobulin-like polypeptide, a cytokine polypeptide, an integrin polypeptide, a signaling lymphocytic activation molecule polypeptide (a SLAM polypeptide), an activating NK cell receptor polypeptide, a BTLA polypeptide, a Toll ligand receptor polypeptide, a CD30 polypeptide, a CDS polypeptide, an ICAM-1 polypeptide, a LFA-1 (CD11a/CD18) polypeptide, a CDS polypeptide, a GITR polypeptide, a BAFFR polypeptide, a HVEM (LIGHTR) polypeptide, a KIRDS2 polypeptide, a SLAMF7 polypeptide, a NKp80 (KLRF1) polypeptide, a NKp44 polypeptide, a NKp30 polypeptide, a NKp46 polypeptide, a CD19 polypeptide, a CD4 polypeptide, a CD8alpha polypeptide, a CD8beta polypeptide, an IL2R beta polypeptide, an IL2R gamma polypeptide, an IL7R alpha polypeptide, an ITGA4 polypeptide, a VLA1 polypeptide, a CD49a polypeptide, an ITGA4 polypeptide, an IA4 polypeptide, a CD49D polypeptide, an ITGA6 polypeptide, a VLA-6 polypeptide, a CD49f polypeptide, an ITGAD polypeptide, a CD11d polypeptide, an ITGAE polypeptide, a CD103 polypeptide, an ITGAL polypeptide, a CD11 a polypeptide, a LFA-1 polypeptide, an ITGAM polypeptide, a CD11b polypeptide, an ITGAX polypeptide, a CD11 c polypeptide, an ITGB1 polypeptide, a CD29 polypeptide, an ITGB2 polypeptide, a CD18 polypeptide, a LFA-1 polypeptide, an ITGB7 polypeptide, a NKG2C polypeptide, a TNFR2 polypeptide, a TRANCE/RANKL polypeptide, a DNAM1 (CD226) polypeptide, a SLAMF4 (CD244, 2B4) polypeptide, a CD84 polypeptide, a CD96 (Tactile) polypeptide, a CEACAM1 polypeptide, a CRTAM polypeptide, a Ly9 (CD229) polypeptide, a CD160 (BY55) polypeptide, a PSGL1 polypeptide, a CD100 (SEMA4D) polypeptide, a CD69 polypeptide, a SLAMF6 (NTB-A, Ly108) polypeptide, a SLAM (SLAMF1, CD150, IPO-3) polypeptide, a BLAME (SLAMF8) polypeptide, a SELPLG (CD162) polypeptide, a LTBR polypeptide, a LAT polypeptide, a GADS polypeptide, a SLP-76 polypeptide, a PAG/Cbp polypeptide, a CD19a polypeptide, and a ligand that specifically binds with CD83, or a combination thereof. A co-stimulatory molecule can bind to a co-stimulatory ligand. Co-stimulatory ligands are proteins expressed on cell surface that upon binding to their receptors produces a co-stimulatory response, i.e., an intracellular response that effects the stimulation provided when an antigen binds to its CAR molecule. Co-stimulatory ligands, include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety. In certain embodiments, the intracellular signaling domain of one of the two or more CARs comprises a co-stimulatory signaling region that comprises a CD28 polypeptide. In certain embodiments, the intracellular signaling domain of one of the two or more CARs comprises a co-stimulatory signaling region that comprises an intracellular domain of CD28 or a portion thereof. In certain embodiments, the intracellular signaling domain of one of the two or more CARs comprises a co-stimulatory signaling region that comprises an intracellular domain of human CD28 or a portion thereof. The CD28 polypeptide can comprise or have an amino acid sequence that is at least about 80%, at least about 85%, at least about 90%, at least about 95%, at least about 96%, at least about 97%, at least about 98%, or at least about 99%, at least about 100% homologous or identical to the sequence having a NCBI Reference No: NP_006130 (SEQ ID NO: 90), or a fragment thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 90 which is at least 20, or at least 30, or at least 40, or at least 50, and up to 220 amino acids in length. Alternatively or additionally, in non-limiting various embodiments, the CD28 polypeptide comprises or has amino acids 1 to 220, 1 to 50, 50 to 100, 100 to 150, 150 to 200, 180 to 219, 180 to 220, or 200 to 220 of SEQ ID NO: 90. In certain embodiments, the intracellular signaling domain of one of the two or more CARs comprises a co-stimulatory signaling region that comprises a CD28 polypeptide comprising or having amino acids 180 to 220 of SEQ ID NO: 90. In certain embodiments, the intracellular signaling domain of one of the two or more CARs comprises a co-stimulatory signaling region that comprises a CD28 polypeptide comprising or having amino acids 180 to 219 of SEQ ID NO: 90.

In certain embodiments, the intracellular signaling domain of one of the two or more CARs comprises a co-stimulatory signaling region that comprises an intracellular domain of mouse CD28 or a portion thereof. In certain embodiments, the CD28 polypeptide comprises or has an amino acid sequence that is at least about 80%, at least about 85%, at least about 90%, at least about 95%, at least about 96%, at least about 97%, at least about 98%, or at least about 99%, at least about 100% homologous or identical to the sequence having a NCBI Reference No: NP_031668.3 (SEQ ID NO: 97), or a fragment thereof, and/or may optionally comprise up to one or up to two or up to three conservative amino acid substitutions. In non-limiting certain embodiments, the CD28 polypeptide comprises or has an amino acid sequence that is a consecutive portion of SEQ ID NO: 97 which is at least about 20, or at least about 30, or at least about 40, or at least about 50, and up to 218 amino acids in length. Alternatively or additionally, in non-limiting various embodiments, the CD28 polypeptide comprises or has amino acids 1 to 218, 1 to 50, 50 to 100, 100 to 150, 150 to 200, 178 to 218, or 200 to 218 of SEQ ID NO: 97. In certain embodiments, the co-stimulatory signaling region of one of the two or more CARs comprises a CD28 polypeptide that comprises or has amino acids 178 to 218 of SEQ ID NO: 97.

SEQ ID NO: 97 is provided below:

[SEQ ID NO: 97]

| | |
|---|---|
| 1 | MTLRLLFLAL NFFSVQVTEN KILVKQSPLL VVDSNEVSLS |
| | CRYSYNLLAK EFRASLYKGV |
| 61 | NSDVEVCVGN GNFTYQPQFR SNAEFNCDGD FDNETVTFRL |
| | WNLHVNHTDI YFCKIEFMYP |
| 121 | PPYLDNERSN GTIIHIKEKH LCHTQSSPKL FWALVVVAGV |
| | LFCYGLLVTV ALCVIWTNSR |
| 181 | RNRLLQSDYM NMTPRRPGLT RKPYQPYAPA RDFAAYRP |

In accordance with the presently disclosed subject matter, a “CD28 nucleic acid molecule” refers to a polynucleotide encoding a CD28 polypeptide. An exemplary nucleotide sequence encoding amino acids 178 to 218 of SEQ ID NO: 97 is set forth in SEQ ID NO: 98, which is provided below.

[SEQ ID NO: 98]
AAT AGTAGAAGGA ACAGACTCCT TCAAAGTGAC TACATGAACA
TGACTCCCCG GAGGCCTGGG CTCACTCGAA AGCCTTACCA
GCCCTACGCC CCTGCCAGAG ACTTTGCAGC GTACCGCCCC

In certain embodiments, the intracellular signaling domain of one of the two or more CARs comprises a intracellular domain of mouse CD28 or a portion thereof. In certain embodiments, the intracellular domain of CD28 or a portion thereof comprises or has an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 99 or a fragment thereof, and/or may optionally comprise up to one or up to two or up to three conservative amino acid substitutions. SEQ ID NO: 99 is provided below:

[SEQ ID NO: 99]
NSRRNRLLQS DYMNMTPRRP GLTRKPYQPY APARDFAAYR P.

An exemplary nucleotide sequence encoding the amino acid sequence of SEQ ID NO: 99 is set forth in SEQ ID NO: 100, which is provided below.

[SEQ ID NO: 100]
AATAGTAGAAGGAACAGACTCCTTCAAAGTGACTACATGAACATGACTCCC
CGGAGGCCTGGGCTCACTCGAAAGCCTTACCAGCCCTACGCCCCTGCCAGA
GACTTTGCAGCGTACCGCCCC

In certain embodiments, the intracellular signaling domain of one of the two or more CARs comprises an intracellular domain of human CD28 or a portion thereof. In certain embodiments, the intracellular domain of human CD28 or a portion thereof comprises or has an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 101 or a fragment thereof, and/or may optionally comprise up to one or up to two or up to three conservative amino acid substitutions. SEQ ID NO: 101 is provided below:

[SEQ ID NO: 101]
RSKRSRLLHS DYMNMTPRRP GPTRKHYQPY APPRDFAAYR S

An exemplary nucleotide sequence encoding the amino acid sequence of SEQ ID NO: 101 is set forth in SEQ ID NO: 102, which is provided below.

[SEQ ID NO: 102]
AGGAGTAAGAGGAGCAGGCTCCTGCACAGTGACTACATGAACATGACTCCC
CGCCGCCCCGGGCCCACCCGCAAGCATTACCAGCCCTATGCCCCACCACGC
GACTTCGCAGCCTATCGCTCC

In certain embodiments, the intracellular signaling domain of one of the two or more CARs comprises a de-immunized intracellular domain of human CD28 or a portion thereof. In certain embodiments, the de-immunized intracellular domain of human CD28 or a portion thereof comprises or has an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 108 or a fragment thereof, and/or may optionally comprise up to one or up to two or up to three conservative amino acid substitutions.

SEQ ID NO: 108 is provided below:

[SEQ ID NO: 108]
RSKRSRLLHS DYMNMTPRRP GPTRKHYQPY APPRDFAAYR K

In certain embodiments, the intracellular signaling domain of one of the two or more CARs comprises a co-stimulatory signaling region that comprises a 4-1BB polypeptide. 4-1BB can act as a tumor necrosis factor (TNF) ligand and have stimulatory activity. In certain embodiments, the 4-1BB polypeptide comprises or has an amino acid sequence that is at least about 80%, at least about 85%, at least about 90%, at least about 95%, at least about 96%, at least about 97%, at least about 98%, or at least about 99%, at least about 100% homologous or identical to the sequence having a NCBI Reference No: NP_001552 (SEQ ID NO: 103) or a fragment thereof, and/or may optionally comprise up to one or up to two or up to three conservative amino acid substitutions. In non-limiting certain embodiments, the 4-1BB polypeptide comprises or has an amino acid sequence that is a consecutive portion of SEQ ID NO: 103 which is at least about 20, or at least about 30, or at least about 40, or at least about 50, and up to 255 amino acids in length. Alternatively or additionally, in non-limiting various embodiments, the 4-1BB polypeptide comprises or has amino acids 1 to 255, 1 to 50, 50 to 100, 100 to 150, 150 to 200, 214 to 255, or 200 to 255 of SEQ ID NO: 103. In certain embodiments, the co-stimulatory signaling region of one of the two or more CARs comprises a 4-1BB polypeptide that comprises or has amino acids 214 to 255 of SEQ ID NO: 103. The amino acid sequence for amino acids 214 to 255 of SEQ ID NO: 103 is SEQ ID NO: 104. SEQ ID NOS: 103 and 104 are provided below.

[SEQ ID NO: 103]

| | |
|---|---|
| 1 | MGNSCYNIVA TLLLVLNFER TRSLQDPCSN CPAGTFCDNN |
| | RNQICSPCPP NSFSSAGGQR |
| 61 | TCDICRQCKG VFRTRKECSS TSNAECDCTP GFHCLGAGCS |
| | MCEQDCKQGQ ELTKKGCKDC |
| 121 | CFGTFNDQKR GICRPWTNCS LDGKSVLVNG TKERDVVCGP |
| | SPADLSPGAS SVTPPAPARE |
| 181 | PGHSPQIISF FLALTSTALL FLLFFLTLRF SVVKRGRKKL |
| | LYIFKQPFMR PVQTTQEEDG |
| 241 | CSCRFPEEEE GGCEL |

[SEQ ID NO: 104]
KRGRKKLLYI FKQPFMRPVQ TTQEEDGCSC RFPEEEEGGC EL

An exemplary nucleotide sequence encoding the amino acid sequence of SEQ ID NO: 104 (or amino acids 214 to 255 of SEQ ID NO: 103) is set forth in SEQ ID NO: 105, which is provided below.

[SEQ ID NO: 105]
AAACGGGGCAGAAAGAAACTCCTGTATATATTCAAACAACCATTTATGAGA
CCAGTACAAACTACTCAAGAGGAAGATGGCTGTAGCTGCCGATTTCCAGAA
GAAGAAGAAGGAGGATGTGAACTG

In certain embodiments, the intracellular signaling domain of one of the two or more CARs comprises a co-stimulatory signaling region that comprises an OX40 polypeptide. In certain embodiments, the OX40 polypeptide comprises or has an amino acid sequence that is at least about 80%, at least about 85%, at least about 90%, at least about 95%, at least about 96%, at least about 97%, at least about 98%, or at least about 99%, at least about 100% homologous or identical to the sequence having a NCBI Reference No: NP_003318 (SEQ ID NO: 106), or a fragment thereof, and/or may optionally comprise up to one or up to two or up to three conservative amino acid substitutions.

SEQ ID NO: 106 is provided below:

[SEQ ID NO: 106]

| | |
|---|---|
| 1 | MCVGARRLGR GPCAALLLLG LGLSTVTGLH CVGDTYPSND |
| | RCCHECRPGN GMVSRCSRSQ |
| 61 | NTVCRPCGPG FYNDVVSSKP CKPCTWCNLR SGSERKQLCT |
| | ATQDTVCRCR AGTQPLDSYK |
| 121 | PGVDCAPCPP GHFSPGDNQA CKPWTNCTLA GKHTLQPASN |
| | SSDAICEDRD PPATQPQETQ |
| 181 | GPPARPITVQ PTEAWPRTSQ GPSTRPVEVP GGRAVAAILG |
| | LGLVLGLLGP LAILLALYLL |
| 241 | RRDQRLPPDA HKPPGGGSFR TPIQEEQADA HSTLAKI |

In accordance with the presently disclosed subject matter, an “OX40 nucleic acid molecule” refers to a polynucleotide encoding an OX40 polypeptide.

In certain embodiments, the intracellular signaling domain of one of the two or more CARs comprises a co-stimulatory signaling region that comprises an ICOS polypeptide. In certain embodiments, the ICOS polypeptide comprises or has an amino acid sequence that is at least about 80%, at least about 85%, at least about 90%, at least about 95%, at least about 96%, at least about 97%, at least about 98%, or at least about 99%, at least about 100% homologous or identical homologous to the sequence having a NCBI Reference No: NP_036224 (SEQ ID NO: 65) or a fragment thereof, and/or may optionally comprise up to one or up to two or up to three conservative amino acid substitutions.

SEQ ID NO: 65 is provided below:

[SEQ ID NO: 65]

| | |
|---|---|
| 1 | MKSGLWYFFL FCLRIKVLTG EINGSANYEM FIFHNGGVQI |
| | LCKYPDIVQQ FKMQLLKGGQ |
| 61 | ILCDLIKTKG SGNTVSIKSL KFCHSQLSNN SVSFFLYNLD |
| | HSHANYYFCN LSIFDPPPFK |
| 121 | VTLIGGYLHI YESQLCCQLK FWLPIGCAAF VVVCILGCIL |
| | ICWLTKKKYS SSVHDPNGEY |
| 181 | MFMRAVNTAK KSRLTDVTL |

In accordance with the presently disclosed subject matter, an “ICOS nucleic acid molecule” refers to a polynucleotide encoding an ICOS polypeptide.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

In certain embodiments, mutation sites and/or junction between domains/motifs/regions of the CAR derived from different proteins are de-immunized. Immunogenicity of junctions between different CAR moieties can be predicted using NetMHC 4.0 Server. For each peptide containing at least one amino acid from next moiety, binding affinity to HLA A, B and C, for all alleles, can be predicted. A score of immunogenicity of each peptide can be assigned for each peptide. Immunogenicity score can be calculated using the formula Immunogenicity score=[(50-binding affinity)*HLA frequency].. n is the number of prediction for each peptide.

Exemplary CAR Constructs

A. 19-28C Construct

In certain embodiments, one of the two or more CARs comprises an extracellular antigen-binding domain that binds to a CD19 polypeptide (e.g., a human CD19 polypeptide), a transmembrane domain and a hinge/spacer region derived from a CD28 polypeptide, an intracellular signaling domain comprising a native CD3ζ polypeptide (e.g., a native human CD3ζ polypeptide) comprising or consisting essentially of or consisting of a native ITAM1, a native ITAM2, a native ITAM3, a native BRS1, a native BRS2, and a native BRS3, and a co-stimulatory signaling region comprising a CD28 polypeptide (e.g., a human CD28 polypeptide). In certain embodiments, the CAR is designated as “19-28ζ”. In certain embodiments, the CAR (e.g., 19-28ζ) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 41, which is provided below. SEQ ID NO: 41 includes a CD8 leader sequence at amino acids 1 to 18, and is able to bind to CD19 (e.g., human CD19).

[SEQ ID NO: 41]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGQNQLYNELNLGRREEYDVL
DKRRGRDPEMGGKPRRKNPQEGLYNELQKDKMAEAYSEIGMKGERRRGKGH
DGLYQGLSTATKDTYDALHMQALPPR

An exemplary nucleotide sequence encoding the amino acid sequence of SEQ ID NO: 41 is set forth in SEQ ID NO: 42, which is provided below.

[SEQ ID NO: 42]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccagaac
cagctctataacgagctcaatctaggacgaagagaggagtacgatgttttg
gacaagagacgtggccgggaccctgagatggggggaaagccgagaaggaag
aaccctcaggaaggcctgtacaatgaactgcagaaagataagatggcggag
gcctacagtgagattgggatgaaaggcgagcgccggaggggcaaggggcac
gatggcctttaccagggtctcagtacagccaccaaggacacctacgacgcc
cttcacatgcaggccctgccccctcgctaa

B. 19-281XX Construct

In certain embodiments, one of the two or more CARs comprises an extracellular antigen-binding domain that binds to a CD19 polypeptide (e.g., a human CD19 polypeptide), a transmembrane domain and a hinge/spacer region derived from a CD28 polypeptide, an intracellular signaling domain comprising a modified CD3ζ polypeptide (e.g., a modified human CD3ζ polypeptide) comprising or consisting essentially of or consisting of a native ITAM1, a native BRS1, a native BRS2, a native BRS3, an ITAM2 variant having two loss-of-function mutations, and an ITAM3 variant having two loss-of-function mutations, and a co-stimulatory signaling region comprising a CD28 polypeptide (e.g., a human CD28 polypeptide). In certain embodiments, the CAR is designated as “19-281XX” or “1XX”. In certain embodiments, the CAR (e.g., 1XX) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 43, which is provided below. SEQ ID NO: 43 includes a CD8 leader sequence at amino acids 1 to 18, and is able to bind to CD19 (e.g., human CD19).

[SEQ ID NO: 43]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGQNQLYNELNLGRREEYDVL
DKRRGRDPEMGGKPRRKNPQEGLFNELQKDKMAEAFSEIGMKGERRRGKGH
DGLFQGLSTATKDTFDALHMQALPPR

An exemplary nucleotide sequence encoding the amino acid sequence of SEQ ID NO: 43 is set forth in SEQ ID NO: 44, which is provided below.

[SEQ ID NO: 44]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccagaac
cagctctataacgagctcaatctaggacgaagagaggagtacgatgttttg
gacaagagacgtggccgggaccctgagatggggggaaagccgagaaggaag
aaccctcaggaaggcctgtTcaatgaactgcagaaagataagatggcggag
gcctTcagtgagattgggatgaaaggcgagcgccggaggggcaaggggcac
gatggcctttTccaggggctcagtacagccaccaaggacacctTcgacgcc
cttcacatgcaggccctgccccctcgctaa

C. 19-28D12 Construct

In certain embodiments, one of the two or more CARs comprises an extracellular antigen-binding domain that binds to a CD19 polypeptide (e.g., a human CD19 polypeptide), a transmembrane domain and a hinge/spacer region derived from a CD28 polypeptide, an intracellular signaling domain comprising a modified CD3ζ polypeptide (e.g., a modified human CD3ζ polypeptide), and a co-stimulatory signaling region comprising a CD28 polypeptide (e.g., a human CD28 polypeptide), wherein the modified CD3ζ polypeptide comprises a native ITAM3 and does not comprise an ITAM1 (native or modified), an ITAM2 (native or modified), a BRS1 (native or modified), a BRS2 (native or modified), or a BRS3 (native or modified). In certain embodiments, the CAR is designated as “19-28D12” or “D12”. In certain embodiments, the CAR (e.g., D12) comprises an amino acid sequence that is at least about 85%, about 90%, about 95%, about 96%, about 97%, about 98%, about 99%, or about 100% homologous to the amino acid sequence set forth in SEQ ID NO: 45, which is provided below. SEQ ID NO: 45 includes a CD8 leader sequence at amino acids 1 to 18, and is able to bind to CD19 (e.g., human CD19).

[SEQ ID NO: 45]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGHDGLYQGLSTATKDTYDAL
HMQALPPR

An exemplary nucleotide sequence encoding the amino acid sequence of SEQ ID NO: 45 is set forth in SEQ ID NO: 46, which is provided below.

[SEQ ID NO: 46]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccacgat
ggcctttaccaggggctcagtacagccaccaaggacacctacgacgccctt
cacatgcaggccctgccccctcgctaa

D. 19-28D23 Construct

In certain embodiments, one of the two or more CARs comprises an extracellular antigen-binding domain that binds to a CD19 polypeptide (e.g., human CD19 polypeptide), a transmembrane domain and a hinge/spacer region derived from a CD28 polypeptide, an intracellular signaling domain comprising a modified CD3ζ polypeptide (e.g., a modified human CD3ζ polypeptide) comprising ITAM1, BRS1 and a deletion of ITAM2, ITAM3, BRS2 and BRS3, and a co-stimulatory signaling region comprising a CD28 polypeptide (e.g., a human CD28 polypeptide), wherein the modified CD3ζ polypeptide comprises a native ITAM1 and a native BRS1, and does not comprise an ITAM2 (native or modified), an ITAM3 (native or modified), a BRS2 (native or modified), or a BRS3 (native or modified). In certain embodiments, the CAR is designated as “19-28D23” or “D23”. In certain embodiments, the CAR (e.g., D23) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 47, which is provided below. SEQ ID NO: 47 includes a CD8 leader sequence at amino acids 1 to 18, and is able to bind to CD19 (e.g., human CD19).

[SEQ ID NO: 47]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGQNQLYNELNLGRREEYDVL
DKRRGRDP

An exemplary nucleotide sequence encoding the amino acid sequence of SEQ ID NO: 47 is set forth in SEQ ID NO: 48, which is provided below.

[SEQ ID NO: 48]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccagaac
cagctctataacgagctcaatctaggacgaagagaggagtacgatgttttg
gacaagagacgtggccgggacccttaa

E. 19-28XX3 Construct

In certain embodiments, one of the two or more CARs comprises an extracellular antigen-binding domain that binds to a CD19 polypeptide (e.g., a human CD19 polypeptide), a transmembrane domain and a hinge/spacer region derived from a CD28 polypeptide, an intracellular signaling domain comprising a modified CD3ζ polypeptide (e.g., a modified human CD3ζ polypeptide) comprising or consisting essentially of or consisting of a native ITAM3, a native BRS1, a native BRS2, a native BRS3, an ITAM1 variant having two loss-of-function mutations, and an ITAM2 variant having two loss-of-function mutations, and a co-stimulatory signaling region comprising a CD28 polypeptide (e.g., a human CD28 polypeptide). In certain embodiments, the CAR is designated as “19-29XX3” or “XX3”. In certain embodiments, the CAR (e.g., XX3) comprises an amino acid sequence that is at least about 85%, about 90%, about 95%, about 96%, about 97%, about 98%, about 99%, or about 100% homologous to the amino acid sequence set forth in SEQ ID NO: 49, which is provided below. SEQ ID NO: 49 includes a CD8 leader sequence at amino acids 1 to 18, and is able to bind to CD19 (e.g., human CD19).

[SEQ ID NO: 49]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGQNQLFNELNLGRREEFDVL
DKRRGRDPEMGGKPRRKNPQEGLFNELQKDKMAEAFSEIGMKGERRRGKGH
DGLYQGLSTATKDTYDALHMQALPPR

An exemplary nucleotide sequence encoding the amino acid sequence of SEQ ID NO: 49 is set forth in SEQ ID NO: 50, which is provided below.

[SEQ ID NO: 50]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccagaac
cagctctTtaacgagctcaatctaggacgaagagaggagtTcgatgttttg
gacaagagacgtggccgggaccctgagatggggggaaagccgagaaggaag
aaccctcaggaaggcctgtTcaatgaactgcagaaagataagatggcggag
gcctTcagtgagattgggatgaaaggcgagcgccggaggggcaaggggcac
gatggcctttaccagggtctcagtacagccaccaaggacacctacgacgcc
cttcacatgcaggccctgccccctcgctaa

F. 19-28X23 Construct

In certain embodiments, one of two or more CARs comprises an extracellular antigen-binding domain that binds to a CD19 polypeptide (e.g., a human CD19 polypeptide), a transmembrane domain and a hinge/spacer region derived from a CD28 polypeptide, an intracellular signaling domain comprising a modified CD3ζ polypeptide (e.g., a modified human CD3ζ polypeptide) comprising or consisting essentially of or consisting of a native ITAM2, a native ITAM3, a native BRS1, a native BRS2, a native BRS3, and an ITAM1 variant having two loss-of-function mutations, and a co-stimulatory signaling region comprising a CD28 polypeptide (e.g., a human CD28 polypeptide). In certain embodiments, the CAR is designated as “19-28X23” or “X23”. In certain embodiments, the CAR (e.g., X23) comprises an amino acid sequence that is at least about 85%, about 90%, about 95%, about 96%, about 97%, about 98%, about 99%, or about 100% homologous to the amino acid sequence set forth in SEQ ID NO: 51, which is provided below. SEQ ID NO: 51 includes a CD8 leader sequence at amino acids 1 to 18, and is able to bind to CD19 (e.g., human CD19).

[SEQ ID NO: 51]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGQNQLFNELNLGRREEFDVL
DKRRGRDPEMGGKPRRKNPQEGLYNELQKDKMAEAYSEIGMKGERRRGKGH
DGLYQGLSTATKDTYDALHMQALPPR

An exemplary nucleotide sequence encoding the amino acid sequence of SEQ ID NO: 51 is set forth in SEQ ID NO: 52, which is provided below.

[SEQ ID NO: 52]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccagaac
cagctctTtaacgagctcaatctaggacgaagagaggagtTcgatgttttg
gacaagagacgtggccgggaccctgagatggggggaaagccgagaaggaag
aaccctcaggaaggcctgtacaatgaactgcagaaagataagatggcggag
gcctacagtgagattgggatgaaaggcgagcgccggaggggcaaggggcac
gatggcctttaccagggtctcagtacagccaccaaggacacctacgacgcc
cttcacatgcaggccctgccccctcgctaa

G. 19-28X2X Construct

In certain embodiments, one of two or more CARs comprises an extracellular antigen-binding domain that binds to a CD19 polypeptide (e.g., a human CD19 polypeptide), a transmembrane domain and a hinge/spacer region derived from a CD28 polypeptide, an intracellular signaling domain comprising a modified CD3ζ polypeptide (e.g., a modified human CD3ζ polypeptide) comprising or consisting essentially of or consisting of a native ITAM2, a native BRS1, a native BRS2, a native BRS3, an ITAM1 variant having two loss-of-function mutations, and an ITAM3 variant having two loss-of-function mutations, and a co-stimulatory signaling region comprising a CD28 polypeptide (e.g., a human CD28 polypeptide). In certain embodiments, the CAR is designated as “19-28X2X” or “X2X”. In certain embodiments, the CAR (e.g., X2X) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 53, which is provided below. SEQ ID NO: 53 includes a CD8 leader sequence at amino acids 1 to 18, and is able to bind to CD19 (e.g., human CD19).

[SEQ ID NO: 53]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGQNQLFNELNLGRREEFDVL
DKRRGRDPEMGGKPRRKNPQEGLYNELQKDKMAEAYSEIGMKGERRRGKGH
DGLFQGLSTATKDTFDALHMQALPPR

An exemplary nucleotide sequence encoding the amino acid sequence of SEQ ID NO: 53 is set forth in SEQ ID NO: 54, which is provided below.

[SEQ ID NO: 54]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccagaac
cagctctTtaacgagctcaatctaggacgaagagaggagtTcgatgttttg
gacaagagacgtggccgggaccctgagatggggggaaagccgagaaggaag
aaccctcaggaaggcctgtacaatgaactgcagaaagataagatggcggag
gcctacagtgagattgggatgaaaggcgagcgccggaggggcaaggggcac
gatggcctttTccaggggctcagtacagccaccaaggacacctTcgacgcc
cttcacatgcaggccctgccccctcgctaa

H. 19-2812X Construct

In certain embodiments, one of the two or more CARs comprises an extracellular antigen-binding domain that binds to a CD19 polypeptide (e.g., a human CD19 polypeptide), a transmembrane domain and a hinge/spacer region derived from a CD28 polypeptide, an intracellular signaling domain comprising a modified CD3ζ polypeptide (e.g., a modified human CD3ζ polypeptide) comprising or consisting essentially of or consisting of a native ITAM1, a native ITAM2, a native BRS1, a native BRS2, a native BRS3, and an ITAM3 variant having two loss-of-function mutations, and a co-stimulatory signaling region comprising a CD28 polypeptide (e.g., a human CD28 polypeptide). In certain embodiments, the CAR is designated as “19-2812X”“12X”. In certain embodiments, the CAR (e.g., 12X) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 55, which is provided below. SEQ ID NO: 55 includes a CD8 leader sequence at amino acids 1 to 18, and is able to bind to CD19 (e.g., human CD19).

[SEQ ID NO: 55]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGQNQLYNELNLGRREEYDVL
DKRRGRDPEMGGKPRRKNPQEGLYNELQKDKMAEAYSEIGMKGERRRGKGH
DGLFQGLSTATKDTFDALHMQALPPR

An exemplary nucleotide sequence encoding the amino acid sequence of SEQ ID NO: 55 is set forth in SEQ ID NO: 56, which is provided below.

[SEQ ID NO: 56]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccagaac
cagctctataacgagctcaatctaggacgaagagaggagtacgatgttttg
gacaagagacgtggccgggaccctgagatggggggaaagccgagaaggaag
aaccctcaggaaggcctgtacaatgaactgcagaaagataagatggcggag
gcctacagtgagattgggatgaaaggcgagcgccggaggggcaaggggcac
gatggcctttTccaggggctcagtacagccaccaaggacacctTcgacgcc
cttcacatgcaggccctgccccctcgctaa

I. 19-28D3 Construct

In certain embodiments, one of the two or more CARs comprises an extracellular antigen-binding domain that binds to a CD19 polypeptide (e.g., human CD19 polypeptide), a transmembrane domain and a hinge/spacer region derived from a CD28 polypeptide, an intracellular signaling domain comprising a modified CD3ζ polypeptide (e.g., a modified human CD3ζ polypeptide) comprising or consisting essentially of or consisting of a native ITAM1, a native ITAM2, a native BRS1, a native BRS2, and a deletion of an ITAM3 and a portion of BRS3, and a co-stimulatory signaling region comprising a CD28 polypeptide (e.g., a human CD28 polypeptide), wherein the modified CD3ζ polypeptide comprises a native ITAM1, a native ITAM2, a native BRS1 and a native BRS2, and does not comprise an ITAM3 (native or modified)or a native BRS3. In certain embodiments, the CAR is designated as “19-28D3” or “D3”. In certain embodiments, the CAR (e.g., D3) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 57, which is provided below. SEQ ID NO: 57 includes a CD8 leader sequence at amino acids 1 to 18, and is able to bind to CD19 (e.g., human CD19).

[SEQ ID NO: 57]
MALPVTALLLPLALLLHAEVKLQQSGAELVRPGSSVKISCKASGYAFSSYW
MNWVKQRPGQGLEWIGQIYPGDGDTNYNGKFKGQATLTADKSSSTAYMQLS
GLTSEDSAVYFCARKTISSVVDFYFDYWGQGTTVTVSSGGGGSGGGGSGGG
GSDIELTQSPKFMSTSVGDRVSVTCKASQNVGTNVAWYQQKPGQSPKPLIY
SATYRNSGVPDRFTGSGSGTDFTLTITNVQSKDLADYFCQQYNRYPYTSGG
GTKLEIKRAAAIEVMYPPPYLDNEKSNGTIIHVKGKHLCPSPLFPGPSKPF
WVLVVVGGVLACYSLLVTVAFIIFWVRSKRSRLLHSDYMNMTPRRPGPTRK
HYQPYAPPRDFAAYRSRVKFSRSADAPAYQQGQNQLYNELNLGRREEYDVL
DKRRGRDPEMGGKPRRKNPQEGLYNELQKDKMAEAYSEIGMKGERRR

An exemplary nucleotide sequence encoding the amino acid sequence of SEQ ID NO: 57 is set forth in SEQ ID NO: 58, which is provided below.

[SEQ ID NO: 58]
atggctctcccagtgactgccctactgcttcccctagcgcttctcctgcat
gcagaggtgaagctgcagcagtctggggctgagctggtgaggcctgggtcc
tcagtgaagatttcctgcaaggcttctggctatgcattcagtagctactgg
atgaactgggtgaagcagaggcctggacagggtcttgagtggattggacag
atttatcctggagatggtgatactaactacaatggaaagttcaagggtcaa
gccacactgactgcagacaaatcctccagcacagcctacatgcagctcagc
ggcctaacatctgaggactctgcggtctatttctgtgcaagaaagaccatt
agttcggtagtagatttctactttgactactggggccaagggaccacggtc
accgtctcctcaggtggaggtggatcaggtggaggtggatctggtggaggt
ggatctgacattgagctcacccagtctccaaaattcatgtccacatcagta
ggagacagggtcagcgtcacctgcaaggccagtcagaatgtgggtactaat
gtagcctggtatcaacagaaaccaggacaatctcctaaaccactgatttac
tcggcaacctaccggaacagtggagtccctgatcgcttcacaggcagtgga
tctgggacagatttcactctcaccatcactaacgtgcagtctaaagacttg
gcagactatttctgtcaacaatataacaggtatccgtacacgtccggaggg
gggaccaagctggagatcaaacgggcggccgcaattgaagttatgtatcct
cctccttacctagacaatgagaagagcaatggaaccattatccatgtgaaa
gggaaacacctttgtccaagtcccctatttcccggaccttctaagcccttt
tgggtgctggtggtggttggtggagtcctggcttgctatagcttgctagta
acagtggcctttattattttctgggtgaggagtaagaggagcaggctcctg
cacagtgactacatgaacatgactccccgccgccccgggcccacccgcaag
cattaccagccctatgccccaccacgcgacttcgcagcctatcgctccaga
gtgaagttcagcaggagcgcagacgcccccgcgtaccagcagggccagaac
cagctctataacgagctcaatctaggacgaagagaggagtacgatgttttg
gacaagagacgtggccgggaccctgagatggggggaaagccgagaaggaag
aaccctcaggaaggcctgtacaatgaactgcagaaagataagatggcggag
gcctacagtgagattgggatgaaaggcgagcgccggaggtaa

J. 19-BBz Construct

In certain embodiments, one of the two or more CARs comprises an extracellular antigen-binding domain that binds to a CD19 polypeptide (e.g., human CD19 polypeptide), a transmembrane domain and a hinge/spacer region derived from a CD8a polypeptide, an intracellular signaling domain comprising a native CD3ζ polypeptide (e.g., a human CD3ζ polypeptide), and a co-stimulatory signaling region comprising a 4-1BB polypeptide (e.g., a human 4-1BB polypeptide). In certain embodiments, the CAR is designated as “19-BBζ”. In certain embodiments, the CAR (e.g., 19-BK) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 118, which is provided below.

[SEQ ID NO: 118]
MALPVTALLLPLALLLHADIELTQSPKFMSTSVGDRVSVTCKASQNVGTNV
AWYQQKPGQSPKPLIYSATYRNSGVPDRFTGSGSGTDFTLTITNVQSKDLA
DYFCQQYNRYPYTSGGGTKLEIKRGGGGSGGGGSGGGGSEVKLQQSGAELV
RPGSSVKISCKASGYAFSSYWMNWVKQRPGQGLEWIGQIYPGDGDTNYNGK
FKGQATLTADKSSSTAYMQLSGLTSEDSAVYFCARKTISSVVDFYFDYWGQ
GTTVTVSSAAAPTTTPAPRPPTPAPTIASQPLSLRPEACRPAAGGAVHTRG
LDFACDIYIWAPLAGTCGVLLLSLVITLYCNKRGRKKLLYIFKQPFMRPVQ
TTQEEDGCSCRFPEEEEGGCELRVKFSRSADAPAYQQGQNQLYNELNLGRR
EEYDVLDKRRGRDPEMGGKPRRKNPQEGLYNELQKDKMAEAYSEIGMKGER
RRGKGHDGLYQGLSTATKDTYDALHMQALPPR

An exemplary nucleotide sequence encoding the amino acid sequence of SEQ ID NO: 118 is set forth in SEQ ID NO: 119, which is provided below.

[SEQ ID NO: 119]
ATGGCTCTCCCAGTGACTGCCCTACTGCTTCCCCTAGCGCTTCTCCTGCAT
GCAGACATTGAGCTCACCCAGTCTCCAAAATTCATGTCCACATCAGTAGGA
GACAGGGTCAGCGTCACCTGCAAGGCCAGTCAGAATGTGGGTACTAATGTA
GCCTGGTATCAACAGAAACCAGGACAATCTCCTAAACCACTGATTTACTCG
GCAACCTACCGGAACAGTGGAGTCCCTGATCGCTTCACAGGCAGTGGATCT
GGGACAGATTTCACTCTCACCATCACTAACGTGCAGTCTAAAGACTTGGCA
GACTATTTCTGTCAACAATATAACAGGTATCCGTACACGTCCGGAGGGGGG
ACCAAGCTGGAGATCAAACGGGGTGGAGGTGGATCAGGTGGAGGTGGATCT
GGTGGAGGTGGATCTGAGGTGAAGCTGCAGCAGTCTGGGGCTGAGCTGGTG
AGGCCTGGGTCCTCAGTGAAGATTTCCTGCAAGGCTTCTGGCTATGCATTC
AGTAGCTACTGGATGAACTGGGTGAAGCAGAGGCCTGGACAGGGTCTTGAG
TGGATTGGACAGATTTATCCTGGAGATGGTGATACTAACTACAATGGAAAG
TTCAAGGGTCAAGCCACACTGACTGCAGACAAATCCTCCAGCACAGCCTAC
ATGCAGCTCAGCGGCCTAACATCTGAGGACTCTGCGGTCTATTTCTGTGCA
AGAAAGACCATTAGTTCGGTAGTAGATTTCTACTTTGACTACTGGGGCCAA
GGGACCACGGTCACCGTCTCCTCAGCggccgcacccaccacgacgccagcg
ccgcgaccaccaacaccggcgcccaccatcgcgtcgcagcccctgtccctg
cgcccagaggcgtgccggccagcggcggggggcgcagtgcacacgaggggg
ctggacttcgcctgtgatatctacatctgggcgcccttggccgggacttgt
ggggtccttctcctgtcactggttatcaccctttactgcaacaaacggggc
agaaagaaactcctgtatatattcaaacaaccatttatgagaccagtacaa
actactcaagaggaagatggctgtagctgccgatttccagaagaagaagaa
ggaggatgtgaactgagagtgaagttcagcaggagcgcagacgcccccgcg
taccagcagggccagaaccagctctataacgagctcaatctaggacgaaga
gaggagtacgatgttttggacaagagacgtggccgggaccctgagatgggg
ggaaagccgagaaggaagaaccctcaggaaggcctgtacaatgaactgcag
aaagataagatggcggaggcctacagtgagattgggatgaaaggcgagcgc
cggaggggcaaggggcacgatggcctttaccagggtctcagtacagccacc
aaggacacctacgacgcccttcacatgcaggccctgCCCCCTCGCTAA

K 22-28z Construct

In certain embodiments, one of the two or more CARs comprises an extracellular antigen-binding domain that binds to a CD22 polypeptide (e.g., human CD22 polypeptide), a transmembrane domain and a hinge/spacer region derived from a CD28 polypeptide, an intracellular signaling domain comprising a native CD3ζ polypeptide (e.g., a human CD3ζ polypeptide), and a co-stimulatory signaling region comprising a CD28 polypeptide (e.g., a human CD28 polypeptide). In certain embodiments, the CAR is designated as “22-28ζ”. In certain embodiments, the CAR (e.g., 22-28ζ) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 120, which is provided below.

[SEQ ID NO: 120]
MELGLSWIFLLAILKGVQCQVQLQQSGPGLVKPSQTLSLTCAISGDSVSSN
SAAWNWIRQSPSRGLEWLGRTYYRSKWYNDYAVSVKSRITINPDTSKNQFS
LQLNSVTPEDTAVYYCAREVTGDLEDAFDIWGQGTMVTVSSGGGGSDIQMT
QSPSSLSASVGDRVTITCRASQTIWSYLNWYQQRPGKAPNLLIYAASSLQS
GVPSRFSGRGSGTDFTLTISSLQAEDFATYYCQQSYSIPQTFGQGTKLEIK
AAAIEVMYPPPYLDNEKSNGTIIHVKGKHLCPSPLFPGPSKPFWVLVVVGG
VLACYSLLVTVAFIIFWVRSKRSRLLHSDYMNMTPRRPGPTRKHYQPYAPP
RDFAAYRSRVKFSRSADAPAYQQGQNQLYNELNLGRREEYDVLDKRRGRDP
EMGGKPRRKNPQEGLYNELQKDKMAEAYSEIGMKGERRRGKGHDGLYQGLS
TATKDTYDALHMQALPPR

An exemplary nucleotide sequence encoding the amino acid sequence of SEQ ID NO: 120 is set forth in SEQ ID NO: 121, which is provided below.

[SEQ ID NO: 121]
aTGGAACTCGGTCTGTCCTGGATTTTTCTGCTTGCGATCTTGAAAGGAGTG
CAGTGCCAAGTACAGCTTCAACAGTCAGGTCCGGGGCTGGTAAAGCCTTCT
CAGACACTCAGCCTGACTTGTGCTATAAGTGGGGATAGTGTTTCATCCAAC
TCAGCGGCGTGGAACTGGATCCGGCAAAGTCCATCTCGGGGCCTGGAGTGG
TTGGGGCGGACCTATTATAGGTCTAAGTGGTACAATGATTACGCCGTCTCA
GTGAAGTCACGGATCACAATCAATCCCGATACGAGTAAGAATCAGTTCTCA
CTTCAGCTTAACAGTGTGACACCTGAAGATACGGCAGTATATTATTGCGCG
AGAGAGGTTACTGGGGACCTCGAAGATGCCTTCGATATCTGGGGTCAAGGC
ACAATGGTTACAGTCAGCTCCGGAGGAGGAGGCAGCGACATACAGATGACA
CAATCTCCGAGTAGCCTTTCCGCATCCGTAGGTGATAGGGTTACCATAACT
TGCCGCGCATCTCAAACGATCTGGTCCTATCTGAACTGGTACCAGCAGAGA
CCAGGAAAAGCTCCTAATCTGCTTATCTACGCCGCAAGCTCACTGCAGTCT
GGGGTTCCGAGTAGATTTTCTGGGCGAGGCAGCGGAACGGATTTTACTCTG
ACCATAAGCTCTCTGCAAGCAGAAGATTTTGCCACGTACTACTGCCAGCAA
TCTTACAGCATCCCACAAACATTTGGACAAGGCACAAAGTTGGAGATCAAA
GCggccgccattgaagttatgtatcctcctccttacctagacaatgagaag
agcaatggaaccattatccatgtgaaagggaaacacctttgtccaagtccc
ctatttcccggaccttctaagcccttttgggtgctggtggtggttggtgga
gtcctggcttgctatagcttgctagtaacagtggcctttattattttctgg
gtgaggagtaagaggagcaggctcctgcacagtgactacatgaacatgact
ccccgccgccccgggcccacccgcaagcattaccagccctatgccccacca
cgcgacttcgcagcctatcgctccagagtgaagttcagcaggagcgcagac
gcccccgcgtaccagcagggccagaaccagctctataacgagctcaatcta
ggacgaagagaggagtacgatgttttggacaagagacgtggccgggaccct
gagatggggggaaagccgagaaggaagaaccctcaggaaggcctgtacaat
gaactgcagaaagataagatggcggaggcctacagtgagattgggatgaaa
ggcgagcgccggaggggcaaggggcacgatggcctttaccagggtctcagt
acagccaccaaggacacctacgacgcccttcacatgcaggccctgccccct
cgctaa

L. 22-BBC Construct

In certain embodiments, one of the two or more CARs comprises an extracellular antigen-binding domain that binds to a CD22 polypeptide (e.g., human CD22 polypeptide), a transmembrane domain and a hinge/spacer region derived from a CD8a polypeptide, an intracellular signaling domain comprising a native CD3ζ polypeptide (e.g., a human CD3ζ polypeptide), and a co-stimulatory signaling region comprising a 4-1BB polypeptide (e.g., a human 4-1BB polypeptide). In certain embodiments, the CAR is designated as “22-BBQ”. In certain embodiments, the CAR (e.g., 22-BK) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 122, which is provided below.

[SEQ ID NO: 122]
MELGLSWIFLLAILKGVQCQVQLQQSGPGLVKPSQTLSLTCAISGDSVSSN
SAAWNWIRQSPSRGLEWLGRTYYRSKWYNDYAVSVKSRITINPDTSKNQFS
LQLNSVTPEDTAVYYCAREVTGDLEDAFDIWGQGTMVTVSSGGGGSDIQMT
QSPSSLSASVGDRVTITCRASQTIWSYLNWYQQRPGKAPNLLIYAASSLQS
GVPSRFSGRGSGTDFTLTISSLQAEDFATYYCQQSYSIPQTFGQGTKLEIK
AAAPTTTPAPRPPTPAPTIASQPLSLRPEACRPAAGGAVHTRGLDFACDIY
IWAPLAGTCGVLLLSLVITLYCNKRGRKKLLYIFKQPFMRPVQTTQEEDGC
SCRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHDG
LYQGLSTATKDTYDALHMQALPPR

An exemplary nucleotide sequence encoding the amino acid sequence of SEQ ID NO: 122 is set forth in SEQ ID NO: 123, which is provided below.

[SEQ ID NO: 123]
ATGGAACTCGGTCTGTCCTGGATTTTTCTGCTTGCGATCTTGAAAGGAGTG
CAGTGCCAAGTACAGCTTCAACAGTCAGGTCCGGGGCTGGTAAAGCCTTCT
CAGACACTCAGCCTGACTTGTGCTATAAGTGGGGATAGTGTTTCATCCAAC
TCAGCGGCGTGGAACTGGATCCGGCAAAGTCCATCTCGGGGCCTGGAGTGG
TTGGGGCGGACCTATTATAGGTCTAAGTGGTACAATGATTACGCCGTCTCA
GTGAAGTCACGGATCACAATCAATCCCGATACGAGTAAGAATCAGTTCTCA
CTTCAGCTTAACAGTGTGACACCTGAAGATACGGCAGTATATTATTGCGCG
AGAGAGGTTACTGGGGACCTCGAAGATGCCTTCGATATCTGGGGTCAAGGC
ACAATGGTTACAGTCAGCTCCGGAGGAGGAGGCAGCGACATACAGATGACA
CAATCTCCGAGTAGCCTTTCCGCATCCGTAGGTGATAGGGTTACCATAACT
TGCCGCGCATCTCAAACGATCTGGTCCTATCTGAACTGGTACCAGCAGAGA
CCAGGAAAAGCTCCTAATCTGCTTATCTACGCCGCAAGCTCACTGCAGTCT
GGGGTTCCGAGTAGATTTTCTGGGCGAGGCAGCGGAACGGATTTTACTCTG
ACCATAAGCTCTCTGCAAGCAGAAGATTTTGCCACGTACTACTGCCAGCAA
TCTTACAGCATCCCACAAACATTTGGACAAGGCACAAAGTTGGAGATCAAA
GCGGCCGCACCCACCACGACGCCAGCGCCGCGACCACCAACACCGGCGCCC
ACCATCGCGTCGCAGCCCCTGTCCCTGCGCCCAGAGGCGTGCCGGCCAGCG
GCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTT
ATCACCCTTTACTGCAACAAACGGGGCAGAAAGAAACTCCTGTATATATTC
AAACAACCATTTATGAGACCAGTACAAACTACTCAAGAGGAAGATGGCTGT
AGCTGCCGATTTCCAGAAGAAGAAGAAGGAGGATGTGAACTGAGAGTGAAG
TTCAGCAGGAGCGCAGACGCCCCCGCGTACCAGCAGGGCCAGAACCAGCTC
TATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCT
CAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGC
CTTTACCAGGGTCTCAGTACAGCCACCAAGGACACCTACGACGCCCTTCAC
ATGCAGGCCCTGTAACCCCCTCGC

M 22-BBC-C Construct

In certain embodiments, one of the two or more CARs comprises an extracellular antigen-binding domain that binds to a CD22 polypeptide (e.g., human CD22 polypeptide), a transmembrane domain and a hinge/spacer region derived from a CD8a polypeptide, an intracellular signaling domain comprising two copies of a native CD3ζ polypeptide (e.g., a human CD3ζ polypeptide), and a co-stimulatory signaling region comprising a 4-1BB polypeptide (e.g., a human 4-1BB polypeptide). The CD3ζ polypeptide has the amino acid sequence set forth in SEQ ID NO: 95. The two copies of the CD3ζ polypeptide are linked by a linker having the amino acid sequence set forth in SEQ ID NO: 66. In certain embodiments, the CAR is designated as “22-BBζ-ζ”. In certain embodiments, the CAR (e.g., 22-BBζ-ζ) comprises an amino acid sequence that is at least about 80%, at least about 85%, at least about 90%, at least about 95%, at least about 96%, at least about 97%, at least about 98%, at least about 99%, or at least about 100% homologous or identical to the amino acid sequence set forth in SEQ ID NO: 147, which is provided below.

[SEQ ID NO: 147]
MELGLSWIFLLAILKGVQCQVQLQQSGPGLVKPSQTLSLTCAISGDSVSSN
SAAWNWIRQSPSRGLEWLGRTYYRSKWYNDYAVSVKSRITINPDTSKNQFS
LQLNSVTPEDTAVYYCAREVTGDLEDAFDIWGQGTMVTVSSGGGGSDIQMT
QSPSSLSASVGDRVTITCRASQTIWSYLNWYQQRPGKAPNLLIYAASSLQS
GVPSRFSGRGSGTDFTLTISSLQAEDFATYYCQQSYSIPQTFGQGTKLEIK
AAAPTTTPAPRPPTPAPTIASQPLSLRPEACRPAAGGAVHTRGLDFACDIY
IWAPLAGTCGVLLLSLVITLYCNKRGRKKLLYIFKQPFMRPVQTTQEEDGC
SCRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHDG
LYQGLSTATKDTYDALHMQALPPRGGGGSGGGGSGGGGSRVKFSRSADAPA
YQQGQNQLYNELNLGRREEYDVLDKRRGRDPEMGGKPRRKNPQEGLYNELQ
KDKMAEAYSEIGMKGERRRGKGHDGLYQGLSTATKDTYDALHMQALPPR

An exemplary nucleotide sequence encoding the amino acid sequence of SEQ ID NO: 147 is set forth in SEQ ID NO: 148, which is provided below.

[SEQ ID NO: 148]
ATGGAACTCGGTCTGTCCTGGATTTTTCTGCTTGCGATCTTGAAAGGAGTG
CAGTGCCAAGTACAGCTTCAACAGTCAGGTCCGGGGCTGGTAAAGCCTTCT
CAGACACTCAGCCTGACTTGTGCTATAAGTGGGGATAGTGTTTCATCCAAC
TCAGCGGCGTGGAACTGGATCCGGCAAAGTCCATCTCGGGGCCTGGAGTGG
TTGGGGCGGACCTATTATAGGTCTAAGTGGTACAATGATTACGCCGTCTCA
GTGAAGTCACGGATCACAATCAATCCCGATACGAGTAAGAATCAGTTCTCA
CTTCAGCTTAACAGTGTGACACCTGAAGATACGGCAGTATATTATTGCGCG
AGAGAGGTTACTGGGGACCTCGAAGATGCCTTCGATATCTGGGGTCAAGGC
ACAATGGTTACAGTCAGCTCCGGAGGAGGAGGCAGCGACATACAGATGACA
CAATCTCCGAGTAGCCTTTCCGCATCCGTAGGTGATAGGGTTACCATAACT
TGCCGCGCATCTCAAACGATCTGGTCCTATCTGAACTGGTACCAGCAGAGA
CCAGGAAAAGCTCCTAATCTGCTTATCTACGCCGCAAGCTCACTGCAGTCT
GGGGTTCCGAGTAGATTTTCTGGGCGAGGCAGCGGAACGGATTTTACTCTG
ACCATAAGCTCTCTGCAAGCAGAAGATTTTGCCACGTACTACTGCCAGCAA
TCTTACAGCATCCCACAAACATTTGGACAAGGCACAAAGTTGGAGATCAAA
GCGGCCGCACCCACCACGACGCCAGCGCCGCGACCACCAACACCGGCGCCC
ACCATCGCGTCGCAGCCCCTGTCCCTGCGCCCAGAGGCGTGCCGGCCAGCG
GCGGGGGGCGCAGTGCACACGAGGGGGCTGGACTTCGCCTGTGATATCTAC
ATCTGGGCGCCCTTGGCCGGGACTTGTGGGGTCCTTCTCCTGTCACTGGTT
ATCACCCTTTACTGCAACAAACGGGGCAGAAAGAAACTCCTGTATATATTC
AAACAACCATTTATGAGACCAGTACAAACTACTCAAGAGGAAGATGGCTGT
AGCTGCCGATTTCCAGAAGAAGAAGAAGGAGGATGTGAACTGagagtgaag
ttcagcaggagcgcagacgcccccgcgtaccagcagggccagaaccagctc
tataacgagctcaatctaggacgaagagaggagtacgatgttttggacaag
agacgtggccgggaccctgagatggggggaaagccgagaaggaagaaccct
caggaaggcctgtacaatgaactgcagaaagataagatggcggaggcctac
agtgagattgggatgaaaggcgagcgccggaggggcaaggggcacgatggc
ctttaccagggtctcagtacagccaccaaggacacctacgacgcccttcac
atgcaggccctgCCCCCTCGCGGTGGGGGTGGTTCCGGGGGAGGCGGCTCA
GGAGGTGGAGGTTCTAGAGTGAAGTTTAGCCGGAGTGCCGATGCTCCAGCC
TATCAGCAAGGACAAAACCAACTTTACAACGAACTTAATCTCGGTAGGCGA
GAGGAATACGATGTGCTTGATAAACGCCGAGGTCGAGATCCCGAAATGGGC
GGGAAACCGCGACGCAAGAATCCTCAAGAAGGACTCTATAATGAGTTGCAG
AAGGACAAGATGGCTGAGGCATATAGTGAGATCGGTATGAAGGGAGAACGG
AGAAGGGGGAAAGGGCATGATGGATTGTACCAAGGACTTAGCACAGCTACT
AAAGATACATATGACGCCCTGCACATGCAAGCATTGCCTCCACGC

In certain embodiments, one of the two or more CARs comprises an intracellular signaling domain comprising a modified CD3ζ polypeptide comprised in 1XX. In certain embodiments, the modified CD3ζ polypeptide comprise or has the amino acid sequence set forth in SEQ ID NO: 135.

In certain embodiments, one of the two or more CARs comprises an intracellular signaling domain comprising a modified CD3ζ polypeptide comprised in D23. In certain embodiments, the modified CD3ζ polypeptide comprise or has the amino acid sequence set forth in SEQ ID NO: 137.

In certain embodiments, one of the two or more CARs comprises an intracellular signaling domain comprising a CD28 polypeptide comprised in 1XX and D23. In certain embodiments, the CD28 polypeptide comprise or has amino acids 180 to 219 of SEQ ID NO: 90. In certain embodiments, the CD28 polypeptide comprise or has the amino acid sequence set forth in SEQ ID NO: 139, which is provided below.

[SEQ ID NO: 139]
RSKRSRLLHSDYMNMTPRRPGPTRKHYQPYAPPRDFAAYRK

An exemplary nucleotide sequence encoding the amino acid sequence of SEQ ID NO: 139 is set forth in SEQ ID NO: 140, which is provided below.

[SEQ ID NO: 140]
aggagtaagaggagcaggctcctgcacagtgactacatgaacatgactccc
cgccgccccgggcccacccgcaagcattaccagccctatgccccaccacgc
gacttcgcagcctatcgcaaa

In certain embodiments, one of the two or more CARs comprises a transmembrane domain and a hinge/spacer region derived from a CD166 polypeptide. In certain embodiments, the hinge/spacer region comprise or has the amino acid sequence set forth SEQ ID NO: 141, which is provided below.

[SEQ ID NO: 141]
NQLERTVNSLNVSAISIPEHDEADEISDENREKVNDQAKLIVGIVVGLLLA
ALVAGVVYWLYMKK

An exemplary nucleotide sequence encoding the amino acid sequence of SEQ ID NO: 141 is set forth in SEQ ID NO: 142, which is provided below.

[SEQ ID NO: 142]
AACCAACTGGAGAGAACAGTAAACTCCTTGAATGTCTCTGCTATAAGTATT
CCAGAACACGATGAGGCAGACGAGATAAGTGATGAAAACAGAGAAAAGGTG
AATGACCAGGCAAAACTAATTGTGGGAATCGTTGTTGGTCTCCTCCTTGCT
GCCCTTGTTGCTGGTGTCGTCTACTGGCTGTACATGAAGAAG

### 7.3. Immunoresponsive Cells

The presently disclosed subject matter provides immunoresponsive cells comprising two or more CARs disclosed herein, wherein the two or more CARs comprise different intracellular signaling domains. In certain embodiments, the CAR is capable of activating the immunoresponsive cell. In certain embodiments, the CAR is expressed from an endogenous locus (e.g., TRAC).

In certain embodiments, compared to cells comprising two or more CARs comprising the same intracellular signaling domains, the presently disclosed immunoresponsive cells have unexpected synergistic effects (e.g., improved therapeutic potency, increased cell accumulation when administered to a subject, decreased cell exhaustion when administered to a subject, and/or higher contingent of memory immune cells when administered to a subject. Immunoresponsive cell exhaustion markers include, but are not limited to, TIM3, LAG3, and PD1. Memory immune cell markers include, but are not limited to, CD62L and CD45RA.

7.3.1. Immunoresponsive Cells Comprising Two or More CARs

In certain embodiments, the immunoresponsive cell comprises two CARs.

In certain embodiments, the immunoresponsive cell comprises: a) a first CAR comprising a first extracellular antigen-binding domain that binds to a first antigen and a first intracellular signaling domain; and b) a second CAR comprising a second extracellular antigen-binding domain that binds to a second antigen and a second intracellular signaling domain, wherein the first intracellular signaling domain is different from the second intracellular signaling domain. In certain embodiments, the first intracellular signaling domain comprises a first co-stimulatory signaling region comprising a first co-stimulatory molecule or a portion thereof, and the second intracellular signaling domain comprises a second co-stimulatory signaling region comprising a second co-stimulatory molecule or a portion thereof, wherein the first co-stimulatory molecule is different from the second co-stimulatory molecule.

In certain embodiments, each of the first and second co-stimulatory molecules is independently selected from the group consisting of a CD28 polypeptide, a 4-1BB polypeptide, an OX40 polypeptide, a DAP-10 polypeptide, a CD27 peptide, a CD40/My88 peptide, a NKGD2 peptide, and combinations thereof In certain embodiments, the first co-stimulatory molecule is a CD28 polypeptide and the second co-stimulatory molecule is selected from the group consisting of a 4-1BB polypeptide, an OX40 polypeptide, an ICOS polypeptide, a DAP-10 polypeptide, a CD27 peptide, a CD40/My88 polypeptide, a NKGD2 polypeptide, a CD2 polypeptide, a CD7 polypeptide, a LIGHT polypeptide, a NKG2C polypeptide, a B7-H3 polypeptide, a FcεRIγ polypeptide, a TNF receptor polypeptide, an Immunoglobulin-like polypeptide, a cytokine polypeptide, an integrin polypeptide, a signaling lymphocytic activation molecule polypeptide (a SLAM polypeptide), an activating NK cell receptor polypeptide, a BTLA polypeptide, a Toll ligand receptor polypeptide, a CD30 polypeptide, a CDS polypeptide, an ICAM-1 polypeptide, a LFA-1 (CDlla/CD18) polypeptide, a CDS polypeptide, a GITR polypeptide, a BAFFR polypeptide, a HVEM (LIGHTR) polypeptide, a KIRDS2 polypeptide, a SLAMF7 polypeptide, a NKp80 (KLRF1) polypeptide, a NKp44 polypeptide, a NKp30 polypeptide, a NKp46 polypeptide, a CD19 polypeptide, a CD4 polypeptide, a CD8alpha polypeptide, a CD8beta polypeptide, an IL2R beta polypeptide, an IL2R gamma polypeptide, an IL7R alpha polypeptide, an ITGA4 polypeptide, a VLA1 polypeptide, a CD49a polypeptide, an ITGA4 polypeptide, an IA4 polypeptide, a CD49D polypeptide, an ITGA6 polypeptide, a VLA-6 polypeptide, a CD49f polypeptide, an ITGAD polypeptide, a CD11d polypeptide, an ITGAE polypeptide, a CD103 polypeptide, an ITGAL polypeptide, a CD11 a polypeptide, a LFA-1 polypeptide, an ITGAM polypeptide, a CD11b polypeptide, an ITGAX polypeptide, a CD11 c polypeptide, an ITGB1 polypeptide, a CD29 polypeptide, an ITGB2 polypeptide, a CD18 polypeptide, a LFA-1 polypeptide, an ITGB7 polypeptide, a NKG2C polypeptide, a TNFR2 polypeptide, a TRANCE/RANKL polypeptide, a DNAM1 (CD226) polypeptide, a SLAMF4 (CD244, 2B4) polypeptide, a CD84 polypeptide, a CD96 (Tactile) polypeptide, a CEACAM1 polypeptide, a CRTAM polypeptide, a Ly9 (CD229) polypeptide, a CD160 (BY55) polypeptide, a PSGL1 polypeptide, a CD100 (SEMA4D) polypeptide, a CD69 polypeptide, a SLAMF6 (NTB-A, Ly108) polypeptide, a SLAM (SLAMF1, CD150, IPO-3) polypeptide, a BLAME (SLAMF8) polypeptide, a SELPLG (CD162) polypeptide, a LTBR polypeptide, a LAT polypeptide, a GADS polypeptide, a SLP-76 polypeptide, a PAG/Cbp polypeptide, a CD19a polypeptide, and a ligand that specifically binds with CD83, and combinations thereof.

In certain embodiments, the first co-stimulatory molecule is a CD28 polypeptide and the second co-stimulatory molecule is a 4-1BB polypeptide.

In certain embodiments, each of the first and second antigens has a high, medium or low density level on the surface of a target cell.

In certain embodiments, an antigen having a low density level has a density of no more than about 5,000 molecules on the surface of a target cell. In certain embodiments, an antigen having a low density level has a density of no more than about 4,000 molecules per cell on the surface of a target cell. In certain embodiments, an antigen having a low density level has a density of no more than about 3,000 molecules per cell on the surface of a target cell. In certain embodiments, an antigen having a low density level has a density of no more than about 2,000 molecules per cell on the surface of a target cell. In certain embodiments, an antigen having a low density level has a density of no more than about 1,000 molecules per cell on the surface of a target cell. In certain embodiments, an antigen having a low density level has a density of between about 1,000 molecules per cell and about 5,000 molecules per cell, between about 1,000 molecules per cell and about 4,000 molecules per cell, between about 1,000 molecules per cell and about 3,000 molecules per cell, between about 2,000 molecules per cell and about 5,000 molecules per cell, between about 2,000 molecules per cell and about 4,000 molecules per cell, between about 2,000 molecules per cell and about 3,000 molecules per cell, or between about 1,000 molecules per cell and about 2,000 molecules per cell on the surface of a target cell. In certain embodiments, an antigen having a low density level has a density of no more than about 50 molecules per cell, no more than about 200 molecules per cell, no more than about 100 molecules per cell, no more than about 50 molecules per cell on the surface of a target cell. In certain embodiments, an antigen having a low density level has a density of between about 50 molecules per cell and about 1000 molecules per cell, between about 100 molecules per cell and about 1000 molecules per cell, between about 500 molecules per cell and about 1000 molecules per cell, between about 50 molecules per cell and about 500 molecules per cell, between about 10 molecules per cell and about 500 molecules per cell, between about 50 molecules per cell and about 800 molecules per cell, between about 100 molecules per cell and about 800 molecules per cell, or between about 200 molecules per cell and about 500 molecules per cell on the surface of a target cell.

In certain embodiments, an antigen having a medium density level has a density of between about 5,000 molecules per cell and about 10,000 molecules per cell. In certain embodiments, an antigen having a medium density level has a density of, between about 5,000 molecules per cell and about 9,000 molecules per cell, between about 5,000 molecules per cell and about 8,000 molecules per cell, between about 5,000 molecules per cell and about 7,000 molecules per cell, between about 6,000 molecules per cell and about 10,000 molecules per cell, between about 6,000 molecules per cell and about 8,000 molecules per cell, or between about 7,000 molecules per cell and about 10,000 molecules per cell on the surface of a target cell.

In certain embodiments, an antigen having a high density level has a density of more than about 10,000 molecules per cell on the surface of a target cell. In certain embodiments, an antigen having a high density level has more than about 20,000 molecules per cell, more than about 30,000 molecules per cell, more than about 40,000 molecules per cell, or more than about 50,000 molecules per cell on the surface of a target cell. In certain embodiments, an antigen having a high density level has a density of between about 10,000 molecules per cell and about 15,000 molecules per cell, between about 10,000 molecules per cell and about 20,000 molecules per cell, between about 10,000 molecules per cell and about 30,000 molecules per cell, between about 10,000 molecules per cell and about 40,000 molecules per cell, between about 10,000 molecules per cell and about 50,000 molecules per cell, between about 20,000 molecules per cell and about 30,000 molecules per cell, between about 20,000 molecules per cell and about 50,000 molecules per cell, or between about 20,000 molecules per cell and about 40000 molecules per cell on the surface of a target cell.

In certain embodiments, the first intracellular signaling domain comprises a native CD3ζ polypeptide. In certain embodiments, the second intracellular signaling domain comprises a native CD3ζ polypeptide.

In certain embodiments, the first intracellular signaling domain comprises a modified CD3ζ polypeptide (e.g., one modified CD3ζ polypeptide disclosed herein). In certain embodiments, the second intracellular signaling domain comprises a modified CD3ζ polypeptide (e.g., one modified CD3ζ polypeptide disclosed herein).

In certain embodiments, non-limiting examples of modified CD3ζ polypeptides include CD3ζ polypeptides comprising one native ITAM, CD3ζ polypeptides comprising two native ITAMs, CD3ζ polypeptides comprising three native ITAMs, CD3ζ polypeptides comprising one ITAM variant disclosed herein, CD3ζ polypeptides comprising two ITAM variants disclosed herein, CD3ζ polypeptides comprising one native BRS region, CD3ζ polypeptides comprising two native BRS regions, CD3ζ polypeptides comprising three native BRS regions, CD3ζ polypeptides that lack all or part of ITAM1, ITAM2, ITAM3 and/or any portion thereof, and any combination thereof.

In certain embodiments, the first intracellular signaling domain is selected from the group consisting of the intracellular signaling domains of 19-28ζ, 1XX, X2X, XX3, X23, 12X, D3, D12, and D23. In certain embodiments, the first intracellular signaling domain is selected from the group consisting of the intracellular signaling domains of 19-28ζ, 1XX, 12X, D3, D12, and D23.

In certain embodiments, the second intracellular signaling domain is selected from the group consisting of the intracellular signaling domains of 22-BBζ, 1XX, X2X, XX3, X23, 12X, D3, D12, D23, and 22-BBζ-ζ. In certain embodiments, the second intracellular signaling domain is selected from the group consisting of the intracellular signaling domains of 22-BBζ, 1XX, 12X, X23, D3, D12, D23, and 22-BBζ-ζ.

In certain embodiments, the first antigen has a medium density level, and the first intracellular signaling domain comprises a modified CD3ζ polypeptide. In certain embodiments, the modified CD3ζ polypeptide comprises or consists essentially of or consists of a native ITAM1, an ITAM2 variant and an ITAM3 variant. In certain embodiments, the native ITAM1 has the amino acid sequence set forth in SEQ ID NO: 23. In certain embodiments, the ITAM2 variant has the amino acid sequence set forth in SEQ ID NO: 29. In certain embodiments, the ITAM3 variant has the amino acid sequence set forth in SEQ ID NO: 33. In certain embodiments, the first intracellular signaling domain is the intracellular signaling domain of 1XX.

In certain embodiments, the first antigen has a high density level, and the first intracellular signaling domain comprises a modified CD3ζ polypeptide. In certain embodiments, the modified CD3ζ polypeptide comprises or consists essentially of or consists of a native ITAM1, an ITAM2 variant and an ITAM3 variant. In certain embodiments, the native ITAM1 has the amino acid sequence set forth in SEQ ID NO: 23. In certain embodiments, the ITAM2 variant has the amino acid sequence set forth in SEQ ID NO: 29. In certain embodiments, the ITAM3 variant has the amino acid sequence set forth in SEQ ID NO: 33. In certain embodiments, the first intracellular signaling domain is the intracellular signaling domain of 1XX.

In certain embodiments, the first antigen has a low density level, and the first intracellular signaling domain comprises a native CD3ζ polypeptide. In certain embodiments, the native CD3ζ polypeptide comprises or has the amino acid sequence set forth in SEQ ID NO: 95. In certain embodiments, the native CD3ζ polypeptide comprises or has amino acids 52 to 164 of SEQ ID NO: 94. In certain embodiments, the first intracellular signaling domain is the intracellular signaling domain of 19-28ζ.

In certain embodiments, the second antigen has a high density level, and the second intracellular signaling domain comprises a modified CD3ζ polypeptide. In certain embodiments, the modified CD3ζ polypeptide comprises or consists essentially of or consists of a native ITAM1, an ITAM2 variant and an ITAM3 variant. In certain embodiments, the native ITAM1 has the amino acid sequence set forth in SEQ ID NO: 23. In certain embodiments, the ITAM2 variant has the amino acid sequence set forth in SEQ ID NO: 29. In certain embodiments, the ITAM3 variant has the amino acid sequence set forth in SEQ ID NO: 33. In certain embodiments, the second intracellular signaling domain is the intracellular signaling domain of 1XX.

In certain embodiments, the second antigen has a high density level, and the second intracellular signaling domain comprises two copies of a CD3ζ polypeptide. In certain embodiments, the CD3ζ polypeptide comprises or has the amino acid sequence set forth in SEQ ID NO: 95. In certain embodiments, the CD3ζ polypeptide comprises or has amino acids 52 to 164 of SEQ ID NO: 94.

In certain embodiments, the second antigen has a medium density level, and the first intracellular signaling domain comprises a native CD3ζ polypeptide. In certain embodiments, the first intracellular signaling domain is the intracellular signaling domain of 22-BBζ.

In certain embodiments, the second antigen has a low density level, and the first intracellular signaling domain comprises a native CD3ζ polypeptide. In certain embodiments, the first intracellular signaling domain is the intracellular signaling domain of 22-BBζ.

Selection of the combination of co-stimulatory signaling regions of the two or more CARs can depend on the densities/surface expression level of the antigens targeted by the CARs.

In certain embodiments, the intracellular signaling domain of a CAR targeting an antigen having a low density level (e.g., the first CAR) comprises a co-stimulatory signaling region that comprises a CD28 polypeptide and a higher number of ITAMs or shorter distance between each ITAM, and said CAR (e.g., the first CAR) can induce a more efficient activation signal against a low-density antigen. In certain embodiments, the intracellular signaling domain of a CAR targeting an antigen having a medium or high density level (e.g., the second CAR) comprises a 4-1BB or other non-CD28 costimulatory domain and a lower number of ITAMs or longer distance between each ITAM, the transmembrane domain of the CAR and/or the co-stimulatory signaling region of the CAR, and said CAR (e.g., the second CAR) can prevent over activation against a medium or high-density antigen.

Non-limiting examples of the combinations of the first and second antigens are provided below:

A. In certain embodiments, the first antigen has a density level of more than about 10,000 molecules per cell on the surface of the target cell, and the second antigen has a density level of more than about 10,000 molecules per cell on the surface of the target cell.

B. In certain embodiments, the first antigen has a density level of between about 5,000 molecules per cell and about 10,000 molecules per cell on the surface of the target cell, and the second antigen has a density level of more than about 10,000 molecules per cell on the surface of the target cell.

C. In certain embodiments, the first antigen has a density level of less than about 5,000 molecules per cell on the surface of the target cell, and the second antigen has a density level of more than about 10,000 molecules per cell on the surface of the target cell.

D. In certain embodiments, the first antigen has a density level of between about 5,000 molecules per cell and about 10,000 molecules per cell on the surface of the target cell, and the second antigen has a density level of between about 5,000 molecules per cell and about 10,000 molecules per cell on the surface of the target cell.

E. In certain embodiments, the first antigen has a density level of less than about 5,000 molecules per cell on the surface of the target cell, and the second antigen has a density level of between about 5,000 molecules per cell and about 10,000 molecules per cell on the surface of the target cell.

F. In certain embodiments, the first antigen has a density level of less than about 5,000 molecules per cell on the surface of the target cell, and the second antigen has a density level of less than about 5,000 molecules per cell on the surface of the target cell.

In any of the above-noted non-limiting example of combinations, the first co-stimulatory molecule is a CD28 polypeptide and the second co-stimulatory molecule is a 4-1BB polypeptide, an OX40 polypeptide, a CD27 polypeptide, or a CD40 polypeptide.

In any one of the above-noted non-limiting examples of combinations, the first signaling domain comprises a native CD3ζ polypeptide or a modified CD3ζ polypeptide (including, but not limited to, the modified CD3ζ polypeptide of the intracellular signaling domains of 1XX CAR, 12X CAR, D23 CAR, D12 CAR, or D3 CAR).

In any one of the above-noted non-limiting examples of combinations, the second signaling domain comprises a native CD3ζ polypeptide or a modified CD3ζ polypeptide (including, but not limited to, the modified CD3ζ polypeptide of the intracellular signaling domains of 1XX CAR, 12X CAR, X23 CAR, D23 CAR, X23 CAR, D12 CAR D3 CAR, CAR).

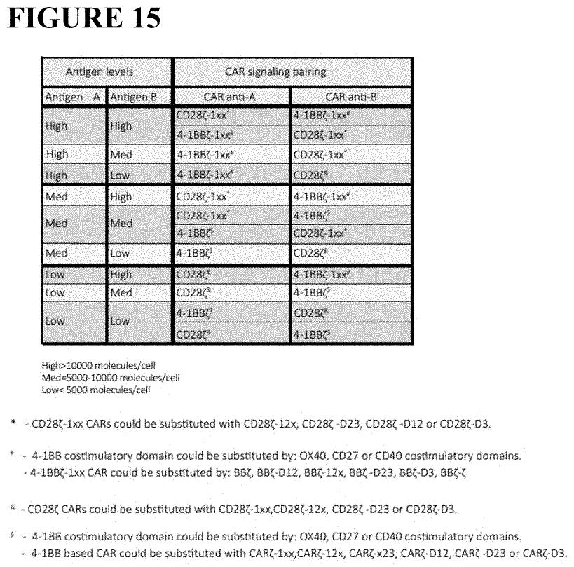

FIG. 15 provides certain embodiments of the two CARs.

In certain embodiments, the first antigen is different from the second antigen. In certain embodiments, each of the first and second antigens is a tumor antigen. In certain embodiments, the first antigen is CD19 and the second antigen is CD22.

In certain embodiments, the immunoresponsive cell comprises three CARs, e.g., the immunoresponsive cell further comprising a third CAR comprising a third extracellular antigen-binding domain that binds to a third antigen and a third intracellular signaling domain. In certain embodiments, the third intracellular signaling domain does not comprise a co-stimulatory signaling region. In certain embodiments, the third intracellular signaling domain comprises a third co-stimulatory signaling region that comprises a third co-stimulatory molecule or a portion thereof In certain embodiments, the third intracellular signaling domain comprises a native CD3ζ polypeptide or a modified CD3ζ polypeptide.

In certain embodiments, the third co-stimulatory molecule is selected from the group consisting of a 4-1BB polypeptide, an OX40 polypeptide, an ICOS polypeptide, a DAP-10 polypeptide, a CD27 polypeptide, a CD40/My88 polypeptide, a NKGD2 polypeptide, a CD2 polypeptide, a CD7 polypeptide, a LIGHT polypeptide, a NKG2C polypeptide, a B7-H3 polypeptide, a FcεRIγ polypeptide, a TNF receptor polypeptide, an Immunoglobulin-like polypeptide, a cytokine polypeptide, an integrin polypeptide, a signaling lymphocytic activation molecule polypeptide (a SLAM polypeptide), an activating NK cell receptor polypeptide, a BTLA polypeptide, a Toll ligand receptor polypeptide, a CD30 polypeptide, a CDS polypeptide, an ICAM-1 polypeptide, a LFA-1 (CD11a/CD18) polypeptide, a CDS polypeptide, a GITR polypeptide, a BAFFR polypeptide, a HVEM (LIGHTR) polypeptide, a KIRDS2 polypeptide, a SLAMF7 polypeptide, a NKp80 (KLRF1) polypeptide, a NKp44 polypeptide, a NKp30 polypeptide, a NKp46 polypeptide, a CD19 polypeptide, a CD4 polypeptide, a CD8alpha polypeptide, a CD8beta polypeptide, an IL2R beta polypeptide, an IL2R gamma polypeptide, an IL7R alpha polypeptide, an ITGA4 polypeptide, a VLA1 polypeptide, a CD49a polypeptide, an ITGA4 polypeptide, an IA4 polypeptide, a CD49D polypeptide, an ITGA6 polypeptide, a VLA-6 polypeptide, a CD49f polypeptide, an ITGAD polypeptide, a CD11d polypeptide, an ITGAE polypeptide, a CD103 polypeptide, an ITGAL polypeptide, a CD11 a polypeptide, a LFA-1 polypeptide, an ITGAM polypeptide, a CD11b polypeptide, an ITGAX polypeptide, a CD11 c polypeptide, an ITGB1 polypeptide, a CD29 polypeptide, an ITGB2 polypeptide, a CD18 polypeptide, a LFA-1 polypeptide, an ITGB7 polypeptide, a NKG2C polypeptide, a TNFR2 polypeptide, a TRANCE/RANKL polypeptide, a DNAM1 (CD226) polypeptide, a SLAMF4 (CD244, 2B4) polypeptide, a CD84 polypeptide, a CD96 (Tactile) polypeptide, a CEACAM1 polypeptide, a CRTAM polypeptide, a Ly9 (CD229) polypeptide, a CD160 (BY55) polypeptide, a PSGL1 polypeptide, a CD100 (SEMA4D) polypeptide, a CD69 polypeptide, a SLAMF6 (NTB-A, Ly108) polypeptide, a SLAM (SLAMF1, CD150, IPO-3) polypeptide, a BLAME (SLAMF8) polypeptide, a SELPLG (CD162) polypeptide, a LTBR polypeptide, a LAT polypeptide, a GADS polypeptide, a SLP-76 polypeptide, a PAG/Cbp polypeptide, a CD19a polypeptide, and a ligand that specifically binds with CD83, and combinations thereof.

In certain embodiments, the third antigen has a low, medium or high density level on the surface of the target cell.

In certain embodiments, the sum of native ITAMs comprised in the three CARs is no more than about five, no more than about four, no more than about three.

The immunoresponsive cells of the presently disclosed subject matter can be cells of the lymphoid lineage. The lymphoid lineage, comprising B, T and natural killer (NK) cells, provides for the production of antibodies, regulation of the cellular immune system, detection of foreign agents in the blood, detection of cells foreign to the host, and the like. Non-limiting examples of immunoresponsive cells of the lymphoid lineage include T cells, Natural Killer (NK) cells, embryonic stem cells, and pluripotent stem cells (e.g., those from which lymphoid cells may be differentiated). T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), Natural killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of a CAR. In certain embodiments, the immunoresponsive cell is a T cell. The T cell can be a CD4⁺ T cell or a CD8⁺ T cell. In certain embodiments, the T cell is a CD4⁺ T cell. In certain embodiments, the T cell is a CD8⁺ T cell.

Natural killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R.A., et al. 2006 Science 314:126-129 (disclosing peripheral donor lymphocytes genetically modified to express a full-length tumor antigen-recognizing T cell receptor complex comprising the α and β heterodimer), in Panelli, M.C., et al. 2000 J Immunol 164:495-504; Panelli, M.C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G.A., et al. 2003 Blood 102:2498-2505 (disclosing selectively in vitro-expanded antigen-specific peripheral blood leukocytes employing artificial antigen-presenting cells (AAPCs) or pulsed dendritic cells). The immunoresponsive cells (e.g., T cells) can be autologous, non-autologous (e.g., allogeneic), or derived in vitro from engineered progenitor or stem cells.

The presently disclosed immunoresponsive cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response. Collectively these immune suppressive factors can induce either marked anergy or apoptosis of adoptively transferred CAR modified T cells upon encounter with targeted tumor cells.

A presently disclosed immunoresponsive cell can further comprise at least one exogenous co-stimulatory ligand, such that the immunoresponsive cell co-expresses or is induced to co-express exogenously the two or more CARs and the at least one exogenous co-stimulatory ligand. The interaction between the two or more CARs and at least one co-stimulatory ligand provides a non-antigen-specific signal important for full activation of an immunoresponsive cell (e.g., T cell). Co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. TNF superfamily members include, without limitation, nerve growth factor (NGF), CD40L (CD40L)/CD154, CD137L/4-1BBL, TNF-α, CD134L/OX40L/CD252, CD27L/CD70, Fas ligand (FasL), CD3OL/CD153, tumor necrosis factor beta (TNFβ)/lymphotoxin-alpha (LTα), lymphotoxin-beta (LTβ), CD257/B cell-activating factor (BAFF)/Blys/THANK/Ta11-1, glucocorticoid-induced TNF Receptor ligand (GITRL), and TNF-related apoptosis-inducing ligand (TRAIL), LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Immunoglobulin superfamily ligands include, without limitation, CD80 and CD86, both ligands for CD28, PD-L1/(B7-H1) that ligands for PD-1.

In certain embodiments, the at least one co-stimulatory ligand is selected from the group consisting of 4-1BBL, CD80, CD86, CD70, OX40L, CD48, TNFRSF14, PD-L1, and combinations thereof. In certain embodiments, the co-stimulatory ligand is 4-1BBL. 4-1BBL can be covalently joined to the N-terminus of the extracellular antigen-binding domain of at least one of the two or more CARs. Alternatively, 4-1BBL can be covalently joined to the C-terminus of the intracellular signaling domain of at least one of the two or more CAR. Cells comprising antigen-binding receptors and one or more co-stimulatory ligand are disclosed in U.S. Patent No. 8,389,282, which is incorporated by reference in its entirety.

The unpurified source of CTLs may be any known in the art, such as the bone marrow, fetal, neonate or adult or other hematopoietic cell source, e.g., fetal liver, peripheral blood or umbilical cord blood. Various techniques can be employed to separate the cells. For instance, negative selection methods can remove non-CTLs initially. mAbs are particularly useful for identifying markers associated with particular cell lineages and/or stages of differentiation for both positive and negative selections.

A large proportion of terminally differentiated cells can be initially removed by a relatively crude separation. For example, magnetic bead separations can be used initially to remove large numbers of irrelevant cells. In certain embodiments, at least about 80%, usually at least 70% of the total hematopoietic cells will be removed prior to cell isolation.

Procedures for separation include, but are not limited to, density gradient centrifugation; resetting; coupling to particles that modify cell density; magnetic separation with antibody-coated magnetic beads; affinity chromatography; cytotoxic agents joined to or used in conjunction with a mAb, including, but not limited to, complement and cytotoxins; and panning with antibody attached to a solid matrix, e.g. plate, chip, elutriation or any other convenient technique.

Techniques for separation and analysis include, but are not limited to, flow cytometry, which can have varying degrees of sophistication, e.g., a plurality of color channels, low angle and obtuse light scattering detecting channels, impedance channels.

The cells can be selected against dead cells, by employing dyes associated with dead cells such as propidium iodide (PI). In certain embodiments, the cells are collected in a medium comprising 2% fetal calf serum (FCS) or 0.2% bovine serum albumin (BSA) or any other suitable, e.g., sterile, isotonic medium.

### 7.4. Nucleic Acid Compositions

The presently disclosed subject matter provides nucleic acid compositions encoding the two or more chimeric antigen receptors (CARs) disclosed herein, e.g., those disclosed in Section 7.2. In certain embodiments, the nucleic acid composition comprises:

a) a first nucleotide sequence encoding a first CAR comprising a first extracellular antigen-binding domain that binds to a first antigen and a first intracellular signaling domain comprising a first co-stimulatory molecule or a portion thereof, wherein the first co-stimulatory molecule is CD28; and

b) a second nucleotide sequence encoding a second CAR comprising a second extracellular antigen-binding domain that binds to a second antigen and a second intracellular signaling domain comprising a second co-stimulatory molecule or a portion thereof, wherein the second co-stimulatory molecule is different from the first co-stimulatory molecule.

The second co-stimulatory molecule can be any one disclosed in Section 7.2.

The nucleic acid composition can be a DNA fragment (e.g., a DNA plasmid) or a RNA fragment.

In certain embodiments, the nucleic acid composition is a vector. The vector can be a viral vector. Non-limiting viral vectors include retroviral vectors, lentiviral vectors, Adeno-associated viruses (AAV) vectors. The vector can also be a non-viral vector. In certain embodiments, the vector is a retroviral vector. In certain embodiments, the non-viral vector is a transposon vector.

The first CAR can be operably linked to a first promoter. The second CAR can be operably linked to a second promoter. The first promoter can be the same as the second promoter. Alternatively, the first promoter is different from the second promoter. The first and the second promoters can be endogenous or exogenous. Non-limiting examples of exogenous promoters include an elongation factor (EF)-1 promoter, a cytomegalovirus immediate-early promoter (CMV) promoter, a simian virus 40 early promoter (SV40) promoter, a phosphoglycerate kinase (PGK) promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

In certain embodiments, the nucleic acid composition is integrated into a T cell. In certain embodiments, the nucleic acid composition is integrated at a locus within the genome of the T cell. Non-limiting examples of the loci include a TRAC locus, a TRBC locus, a TRDC locus, and a TRGC locus. In certain embodiments, the locus is a TRAC locus or a TRBC locus. Methods of targeting a CAR to a site within the genome of T cell are disclosed in W02017180989 and Eyquem et al., Nature. (2017 Mar 2); 543(7643): 113-117, both of which are incorporated by reference in their entireties.

Genetic modification of an immunoresponsive cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide encoding a CAR can be cloned into a retroviral vector and expression can be driven from its endogenous promoter, from the retroviral long terminal repeat, or from a promoter specific for a target cell type of interest. Non-viral vectors may be used as well.

For initial genetic modification of an immunoresponsive cell to include a CAR, a retroviral vector is generally employed for transduction, however any other suitable viral vector or non-viral delivery system can be used. The CAR can be constructed with an auxiliary molecule (e.g., a cytokine) in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF -1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides , e.g., P2A, T2A, E2A and F2A peptides). In certain embodiments, any vector or CAR disclosed herein can comprise a P2A peptide comprising the amino acid sequence of GSGATNFSLLKQAGDVEENPGP (SEQ ID NO: 107). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify an immunoresponsive cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S- 83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of an immunoresponsive cell. For example, a nucleic acid molecule can be introduced into an immunoresponsive cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor la enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

### 7.6. Genome Editing Methods

Any targeted genome editing methods can be used to place two or more CARs at one or more endogenous gene loci of a presently disclosed immunoresponsive cell. In certain embodiments, a CRISPR system is used to deliver two or more CARs to one or more endogenous gene loci of a presently disclosed immunoresponsive cell. In certain embodiments, zinc-finger nucleases are used to deliver two or more CARs to one or more endogenous gene loci of a presently disclosed immunoresponsive cell. In certain embodiments, a TALEN system is used to deliver two or more CARs to one or more endogenous gene loci of a presently disclosed immunoresponsive cell.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Placement of two or more CAR can be made at any endogenous gene locus. In certain embodiments, the endogenous gene locus is a TRAC locus, a TRBC locus or a TRGC locus. In certain embodiments, the endogenous gene locus is a TRAC locus. In certain embodiments, the placement of the CAR disrupts or abolishes the endogenous expression of a TCR.

### 7.7. Polypeptides and Analogs

Also included in the presently disclosed subject matter are a CD19, CD8, CD28, CD3ζ, CD40, 4-1BB, OX40, CD84, CD166, CD8a, CD8b, ICOS, ICAM-1, CD27, MY88, NKGD2 and CTLA-4 polypeptides or fragments thereof that are modified in ways that enhance their anti-neoplastic activity when expressed in an immunoresponsive cell. The presently disclosed subject matter provides methods for optimizing an amino acid sequence or nucleic acid sequence by producing an alteration in the sequence. Such alterations may include certain mutations, deletions, insertions, or post-translational modifications. The presently disclosed subject matter further includes analogs of any naturally-occurring polypeptide disclosed herein (including, but not limited to, CD19, CD8, CD28, CD3ζ, CD40, 4-1BB, OX40, CD27, CD40/My88, NKGD2, CD84, CD166, CD8a, CD8b, ICOS, ICAM-1, and CTLA-4). Analogs can differ from a naturally-occurring polypeptide disclosed herein by amino acid sequence differences, by post-translational modifications, or by both. Analogs can exhibit at least about 85%, about 90%, about 91%, about 92%, about 93%, about 94%, about 95%, about 96%, about 97%, about 98%, about 99% or more homologous to all or part of a naturally-occurring amino, acid sequence of the presently disclosed subject matter. The length of sequence comparison is at least 5, 10, 15 or 20 amino acid residues, e.g., at least 25, 50, or 75 amino acid residues, or more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amina acids, e.g., D-amino acids or non-naturally occurring or synthetic amino acids, e.g., .beta. or .gamma. amino acids.

In addition to full-length polypeptides, the presently disclosed subject matter also provides fragments of any one of the polypeptides or peptide domains disclosed herein. As used herein, the term “a fragment” means at least 5, 10, 13, or 15 amino acids. In certain embodiments, a fragment comprises at least 20 contiguous amino acids, at least 30 contiguous amino acids, or at least 50 contiguous amino acids. In certain embodiments, a fragment comprises at least 60 to 80, 100, 200, 300 or more contiguous amino acids. Fragments can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein analogs have a chemical structure designed to mimic the functional activity of a protein disclosed herein. Such analogs may exceed the physiological activity of the original polypeptide. Methods of analog design are well known in the art, and synthesis of analogs can be carried out according to such methods by modifying the chemical structures such that the resultant analogs increase the anti-neoplastic activity of the original polypeptide when expressed in an immunoresponsive cell. These chemical modifications include, but are not limited to, substituting alternative R groups and varying the degree of saturation at specific carbon atoms of a reference polypeptide. In certain embodiments, the protein analogs are relatively resistant to in vivo degradation, resulting in a more prolonged therapeutic effect upon administration. Assays for measuring functional activity include, but are not limited to, those described in the Examples below.

### 7.8. Administration

Compositions comprising the presently disclosed immunoresponsive cells can be provided systemically or directly to a subject for inducing and/or enhancing an immune response to an antigen and/or treating and/or preventing a neoplasm, pathogen infection, or infectious disease. In certain embodiments, the presently disclosed immunoresponsive cells or compositions comprising thereof are directly injected into an organ of interest (e.g., an organ affected by a neoplasia). Alternatively, the presently disclosed immunoresponsive cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of T cells, NK cells, or CTL cells in vitro or in vivo.

The presently disclosed immunoresponsive cells can be administered in any physiologically acceptable vehicle, normally intravascularly, although they may also be introduced into bone or other convenient site where the cells may find an appropriate site for regeneration and differentiation (e.g., thymus). Usually, at least about 1×10⁵ cells will be administered, eventually reaching about 1×10¹⁰ or more. The presently disclosed immunoresponsive cells can comprise a purified population of cells. Those skilled in the art can readily determine the percentage of the presently disclosed immunoresponsive cells in a population using various well-known methods, such as fluorescence activated cell sorting (FACS). Suitable ranges of purity in populations comprising the presently disclosed immunoresponsive cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed immunoresponsive cells or their progenitors and a pharmaceutically acceptable carrier. Administration can be autologous or heterologous. For example, immunoresponsive cells, or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed immunoresponsive cell), it can be formulated in a unit dosage injectable form (solution, suspension, emulsion).

### 7.9. Formulations

Compositions comprising the presently disclosed immunoresponsive cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified immunoresponsive cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON′S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified immunoresponsive cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10⁴ and about 10¹⁰, between about 10⁵ and about 10⁹, or between about 10⁶ and about 10⁸ of the presently disclosed immunoresponsive cells are administered to a human subject. More effective cells may be administered in even smaller numbers. In certain embodiments, at least about 1×10⁵, about 5×10⁵, about 1×10⁶, about 5×10⁷, about 1×10⁷, about 5×10⁷, about 1×10⁸, about 2×10⁸, about 3×10⁸, about 4×10⁸, or about 5×10⁸ of the presently disclosed immunoresponsive cells are administered to a human subject. In certain embodiments, between about 1×10⁵ and 1×10⁶ of the presently disclosed immunoresponsive cells are administered to a human subject. In certain embodiments, between about 1×10⁵ and 5×10⁵ of the presently disclosed immunoresponsive cells are administered to a human subject. In certain embodiments, between about 1×10⁷ and 5×10⁸ of the presently disclosed immunoresponsive cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 7.10. Methods of Treatment

The presently disclosed subject matter provides methods for inducing and/or increasing an immune response in a subject in need thereof. The presently disclosed immunoresponsive cells and compositions comprising thereof can be used for treating and/or preventing a neoplasm in a subject. The presently disclosed immunoresponsive cells and compositions comprising thereof can be used for prolonging the survival of a subject suffering from a neoplasm. The presently disclosed immunoresponsive cells and compositions comprising thereof can also be used for treating and/or preventing a pathogen infection or other infectious disease in a subject, such as an immunocompromised human subject. The presently disclosed immunoresponsive cells and compositions comprising thereof can also be used for reducing tumor burden in a subject. In certain embodiments, the presently disclosed immunoresponsive cells and compositions comprising thereof reduces the number of tumor cells, reduces tumor size, and/or eradicates the tumor in the subject.

In certain embodiments, the presently disclosed immunoresponsive cells can be used to treat a subject having a relapse of a disease. In certain embodiments, the subject received an immunotherapy prior to said administration of the presently disclosed immunoresponsive cells. In certain embodiments, the subject received immunoresponsive cells (e.g., T cells) comprising a CAR comprising an intracellular signaling domain that comprises a co-stimulatory signaling region comprising a 4-1BB polypeptide (e.g., a 4-1BBζ CAR) prior to said administration of the presently disclosed immunoresponsive cells.

In certain embodiments, the subject received treatment that leads to residual tumor cells. In certain embodiments, the residual tumor cells have a low density of a target molecule on the surface of the tumor cells. In certain embodiments, a target molecule having a low density on the cell surface has below about 10000 molecules per cell, below about 8000 molecules per cell, below about 6000 molecules per cell, below about 4000 molecules per cell, below about 2000 molecules per cell, below about 1000 molecules per cell, below about 500 molecules per cell, below about 200 molecules per cell, or below about 100 molecules per cell, In certain embodiments, a target molecule having a low density on the cell surface has between about 4000 to about 2000 molecules per cell or between about 2000 to about 1000 molecules per cell.

In certain embodiments, the tumor cells have a low density of a tumor specific antigen on the surface of the tumor cells. In certain embodiments, the disease is CD19⁺ ALL. In certain embodiments, the tumor cells have a low density of CD19 on the tumor cells.

Such methods comprise administering the presently disclosed immunoresponsive cells in an amount effective or a composition (e.g., pharmaceutical composition) comprising thereof to achieve the desired effect, alleviation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the immunoresponsive cells administered.

For adoptive immunotherapy using antigen-specific T cells, cell doses in the range of about 10⁶-10¹⁰ (e.g., about 10) are typically infused. Upon administration of the presently disclosed cells into the host and subsequent differentiation, T cells are induced that are specifically directed against the specific antigen. The immunoresponsive cells can be administered by any method known in the art including, but not limited to, intravenous, subcutaneous, intranodal, intratumoral, intrathecal, intrapleural, intraperitoneal and directly to the thymus.

The presently disclosed subject matter provides methods for treating and/or preventing a neoplasm in a subject. The method can comprise administering an effective amount of the presently disclosed immunoresponsive cells or a composition comprising thereof to a subject having a neoplasm.

Non-limiting examples of neoplasms include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolarcarcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed immunoresponsive cells and compositions comprising thereof can be used for treating and/or preventing blood cancers (e.g., leukemias, lymphomas, and myelomas) or ovarian cancer, which are not amenable to conventional therapeutic interventions.

In certain embodiments, the neoplasm or tumor is selected from the group consisting of blood cancer, B cell leukemia, multiple myeloma, Acute Myeloid Leukemia (AML), acute lymphoblastic leukemia (ALL), chronic lymphocytic leukemia, non-Hodgkin's lymphoma, and ovarian cancer. In certain embodiments, the first antigen is CD19. In certain embodiments, the first antigen is BCMA or ADGRE2.

In certain embodiments, the neoplasm or tumor is a solid tumor. In certain embodiments, the first antigen is mesothelin (MSLN) or PSMA.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement includes decreased risk or rate of progression or reduction in pathological consequences of the tumor.

A second group of suitable subjects is known in the art as the “adjuvant group.” These are individuals who have had a history of neoplasia, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasia. Features typical of high-risk subgroups are those in which the tumor has invaded neighboring tissues, or who show involvement of lymph nodes.

Another group have a genetic predisposition to neoplasia but have not yet evidenced clinical signs of neoplasia. For instance, women testing positive for a genetic mutation associated with breast cancer, but still of childbearing age, can wish to receive one or more of the immunoresponsive cells described herein in treatment prophylactically to prevent the occurrence of neoplasia until it is suitable to perform preventive surgery.

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed immunoresponsive cells or a composition comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

Further modification can be introduced to the presently disclosed immunoresponsive cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed immunoresponsive cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of a presently disclosed CAR. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells. The incorporation of a suicide gene into the a presently disclosed CAR gives an added level of safety with the ability to eliminate the majority of CART cells within a very short time period. A presently disclosed immunoresponsive cell (e.g., a T cell) incorporated with a suicide gene can be pre-emptively eliminated at a given timepoint post CAR T cell infusion, or eradicated at the earliest signs of toxicity.

### 7.11. Kits

The presently disclosed subject matter provides kits for inducing and/or enhancing an immune response and/or treating and/or preventing a neoplasm or a pathogen infection in a subject. In certain embodiments, the kit comprises an effective amount of presently disclosed immunoresponsive cells or a pharmaceutical composition comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding a presently disclosed CAR that is directed toward an antigen of interest in expessible form, which may optionally be comprised in one or more vectors.

If desired, the immunoresponsive cells and/or nucleic acid molecules are provided together with instructions for administering the cells or nucleic acid molecules to a subject having or at risk of developing a neoplasia or pathogen or immune disorder. The instructions generally include information about the use of the composition for the treatment and/or prevention of a neoplasm or a pathogen infection. In certain embodiments, the instructions include at least one of the following: description of the therapeutic agent; dosage schedule and administration for treatment or prevention of a neoplasia, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 8. EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

### Introduction

CARs are synthetic receptors for antigen that reprogram T cell specificity, function and persistencel. Patient-derived CAR T cells have demonstrated remarkable efficacy against a range of B cell malignancies^{1,2,3}, and early trial results suggest activity in multiple myeloma⁴. Despite high complete response rates, relapses occur in a large fraction of patients, some of which are antigen-negative and others antigen-low^{1,2,4,5,6,7,8}. Whereas some mechanisms resulting in complete and permanent antigen loss have been identified^{6,9,8,10}, those leading to antigen-low tumour escape remain obscure. In murine leukaemia models, CARs provoked reversible antigen loss through trogocytosis, an active mechanism resulting in transfer of the target antigen to T cells. CAR target trogocytosis not only results in decreased target density but also diminishes CAR T cell activity by promoting fratricide killing and exhaustion. These mechanisms affect both CD28 and 4-1BB-based CARs albeit differentially, depending on antigen density, cooperative killing and combinatorial targeting. Antigen loss can also be irreversible. The co-targetting strategies described herein can rescue from reversible and irreversible antigen escape.

### Materials and Methods

Cell culture: NIH/3T3, NALM6, SUP-B15, Raji, SK-OV-3 and A549 cell lines were obtained from ATCC. KMS-12-BM cell line was obtained from DSMZ. NALM6, SUP-B15 and Raji cell lines were cultured in RPMI-1640 (Invitrogen) supplemented with 10% FBS (HyClone), 10 mM HEPES (Invitrogen), L-glutamine 2 mM (Invitrogen), NEAA 1×(Invitrogen), 0.55 mM β-mercaptoethanol, 1 mM sodium pyruvate (Invitrogen). KMS-BM-12 cells were cultured in RPMI-1640 supplemented with 20% FBS. SK-OV-3 and A549 cells were cultured in DMEM (Invitrogen) supplemented with 10% FBS. NIH-3T3 cells were cultured with DMEM supplemented with 10% FCS (HyClone). For proteomics quantification experiments, NALM6 cells transduced to express CD19-mCherry fusion protein were grown in RPMI 1640 containing stable isotope ¹³C₆ L-lysin (SILAC Protein Quantitation Kit-RPMI 1640, Thermo Scientific) for 7 days before processing to co-culture with CAR T cells. NALM6 cells were transduced with firefly luciferase-GFP to allow in vivo tumour burden imaging as described. SK-OV-3 cells were transduced to express CD19. NIH/3T3 cells expressing human CD19 served as alternative antigen presenting cells in proliferation assays. All cells were routinely tested for mycoplasma contamination using the MycoAlert Mycoplasma Detection Kit (Lonza).

Vector constructs: 19-28ζ-LNGFR, 19-BBζ-LNGFR CARs contain the SJ25C1 or FMC63 CD19-specific scFv^{13,15}. 19del-LNGFR, a CAR construct lacking co-stimulatory and ζ chain signalling domains, contain the SJ25C1 CD19-specific scFv. CD22-28ζ-DsRed, CD22-BBζ-DsRed CARs contain the m971 CD22-specific scFv^{7,20,21}. The sequences of m971scFv are provided in Table 2. BCMA-BBζ-DsRed CAR contain the 11D5-3 BCMA-specific scFv⁴. MSLN-BBζ-DsRed CAR contain the m912 MSLN-scpecific scFv²⁰. CAR cDNAs were cloned in the SFG γ-retroviral vector. CD19 cDNA was cloned in SFG γ-retroviral or pLM-Lentiviral vector backbone under the control of the PGK-100 promoter. The CD19-mCherry fusion was generated by fusing the mCherry sequence at the carboxyterminus of CD19 via a GS linker and cloned into the pLM-Lentiviral vector backbone. The CAR-GFP fusion has been previously described²¹. All constructs were prepared using standard molecular biology techniques. Viral supernatants were prepared as previously described²¹.

T cells activation and transduction: Buffy coats from healthy donors were obtained from the New York Blood Center. PBMCs were isolated by density gradient centrifugation and activated as previously described¹³. Purified PBMCs were cultured in RPMI-1640 media supplemented with 10% FBS. Forty-eight hours after activation, T cells were transduced with retroviral supernatants by centrifugation on Retronectin (Takara)-coated plates. Apheresis product and blood and bone marrow samples pre- and post- CAR T cell infusion were obtained from patients consented and enrolled in phase I 19-28ζ CAR T cell clinical trials approved by the MSKCC Institutional Review Board (IRB). Clinical trials are registered under identifiers NCT01044069 and NCT00466531 at ClinicalTrials.gov website. Patients CAR T cells have been manufactured as previously described^{16,22}.

T cell expansion: 3e5 irradiated NIH/3T3 cells expressing human CD19 were plated on 24-well plates and used to stimulate 1e6 CAR T cells/ml as previously described¹². Cells were maintained in RPMI-1640 media supplemented with 10% FBS without addition of cytokines. For in vitro assays, CAR T cells were bead-sorted four days after T cell transduction and before co-culture with NIH/3T3 cells. CAR T cells were stained with LNGFR-PE (clone C40-1457, BD antibody followed by co-incubation with anti-PE beads (Miltenyi). All in vitro assays were performed 7 days after stimulation on NIH/3T3-CD19+ cells. T cells were enumerated using an automated cell counter (Nexcelom Bioscience).

Mouse systemic tumour model: Male or female, 8-12 week old NOD.Cg-Prkdc^{scid}I12rg^{tmWjl}/SzJ (NSG) mice (Jackson Laboratory), were used, under a protocol approved by the MSKCC Institutional Animal Care and Use Committee, according to all relevant animal use guidelines and ethical regulations. 0.5e6 FFLuc-GFP NALM6 cells were administered intravenously (i.v.) by tail vein injection (Day -4). Four days later, 1e6, 0.4 e6 or 0.2e6, CAR T cells were administered i.v. by tail vein injection (Day 0). In some experiments, a second CAR T cell infusion was administered intravenously as indicated in the text. All in vivo assays were performed with bulk transduced CAR T cells except dual antigen targeting experiments. CAR T cells co-transduced with anti-CD19 CAR and anti-CD22 CAR were bead-sorted- based on anti-CD19-CAR expression. Tumour burden was measured by Bioluminescence imaging used the Xenogen IVIS Imaging System (Xenogen). Living Image software (Xenogen) was used to analyse acquired bioluminescence data.

Flow cytometry: All antibodies were titrated. CAR expression was measured with Alexa-fluor 647-conjugated goat anti-mouse Fab (Jackson ImmunoResearch) or Biotinylated Protein L (Thermo Scientific) followed by BV510-stretpatvidin (BD). Primary cells and cell lines phenotype was determined using the following anti-human antibodies: CD19-PE, -BUV395 (clone SJ25C1, BD), CD19-BV510 (clone SJ25C1, Biolegend), CD22-PE (clone S-HCL-1, BD), CD22-BV421 (Clone HIB22, BD), CD81-BV605 (clone JS-81, BD), BCMA-BV421 (clone 19F2, BD), MSLN-Alexa-fluor 700 (clone 420411, R&D system), CD3-BUV737 (clone UCHT, BD), LNGFR-PE, -PE-cy7, -AF647 (clone C40-1457, BD), PD-1-BV711 (clone EH121, BD), LAG-3-BV650 (clone 11C3C65, Biolegend), TIM-3-BV785 (clone F38-2E2, Biolegend), T-bet-AF647 (clone 4B10, Biolegend), EOMES-PE (clone WD1928, ebioscience), Ki67-PEcy7 (clone B56, BD), CD45-BV711 (Clone HI30, BD), CD5-BB515 (clone UCHT2, BD) and CD10-PE (clone HI10a, BD). Countbright beads (Invitrogen) were used to determine the absolute number of cells according to the manufacturer's protocol. 7-AAD or DAPI was used to exclude dead cells. For fixed cells, eFluor506 fixable viability dye (eBioscience) was used. Fc Receptor Binding Inhibitor Antibody Human (eBioscience) and Fc block Mouse (Miltenyi) were used to block Fc receptors. For intracellular staining, cells were fixed and permeabilized using Intracellular Fixation and Permeabilisation Buffer set (eBioscence) according to the manufacturer's protocol. Phycoerythrin Fluorescence Quantitation Kit (BD) was used according to the manufacturer's protocol to determine the number of CD19 and CD22 molecules/cell. Data were collected using BD LSR-II and BD LSR-Fortessa cytometer. Data were analyzed with FlowJo Software (Treestar). Cell sorting was performed by a BD FACSAria cell sorter.

Flow Cytometric trogocytosis assay: 1e5 CAR T cells were co-cultured with target cells in 96-well plates at a 1:1 ratio. After 1, 2 or 4 hours of co-culture at 37° C., cells were washed with FACS buffer containing PBS, 0.5 mM EDTA and 0.5% BSA. After the last wash, cells were suspended in 50μL of FACS buffer and stained with antibodies. Cells were incubated with staining antibodies for 30 minutes at 4° C. Following staining, cells were washed and then analysed by flow cytometry. 7-AAD or DAPI was used to exclude dead cells. Trogocytosis was measured by the surface loss of antigen on target cells and its acquisition on CAR T cells. For trogocytosis inhibition assays, CAR T cells were pretreated with 1pM of Latrunculin A (Sigma-Aldrich) at 37° C. for 15 minutes before co-incubation with target cells.

Cytotoxicity assays: Bulk cytotoxicity of CAR T cells was determined by standard chromium release (⁵¹Cr) assay or luciferase-based assay. For ⁵¹Cr release assay, T cells expressing CD19 were loaded with ⁵¹Cr (PerkinElmer) for 1 h at 37° C. CD19+T cells were cultured with CAR T cells per well in 96-well plates at different effector:target cell (E:T) ratios for 4 h. Specific 51Cr release was calculated using the formula (⁵¹Cr release—spontaneous release)/(maximum release—spontaneous release)×100. For luciferase-based cytotoxic assay, NALM6 expressing FFluc-GFP were co-cultured in 96-well plates with CAR T cells at different effector:target cell (E:T) ratios for 18h. Target cells alone were used to determine maximal luciferase expression (relative light units; RLUmax). After 18 hours of co-culture, 50 μl luciferase substrate (Bright-Glo, Promega) was added to each well. Emitted light was detected in a luminescence plate reader or Xenogen IVIS Imaging System (Xenogen), and quantified using Living Image software (Xenogen). Lysis was determined as (1−(RLUsample)/(RLU))×100.

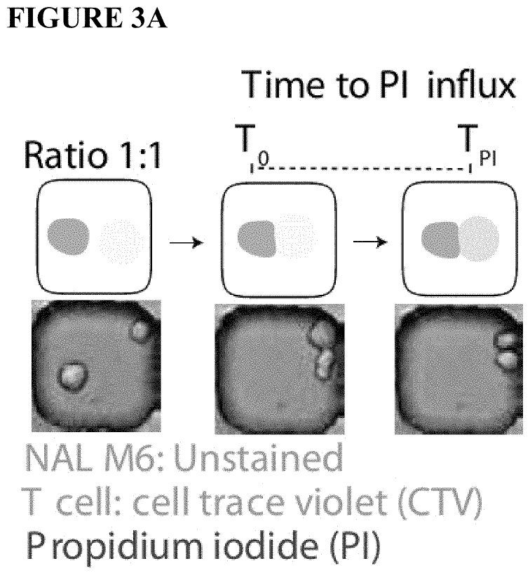

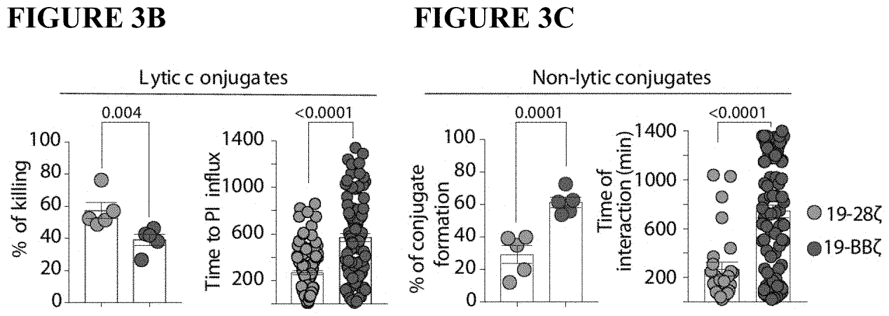

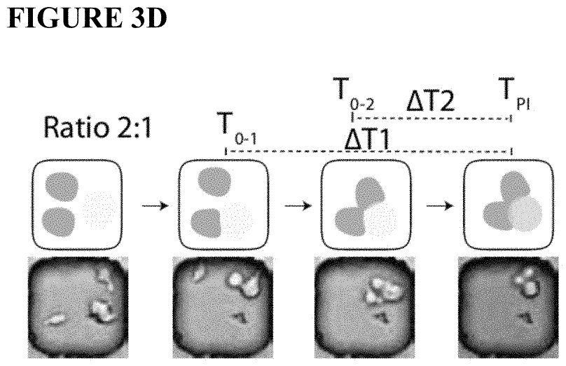

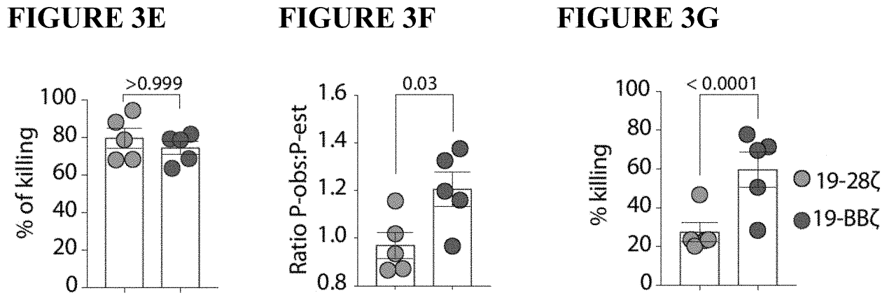

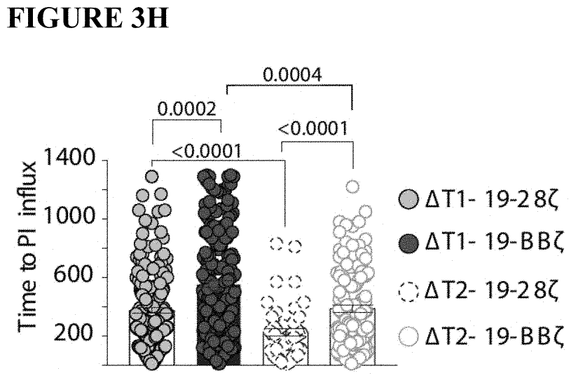

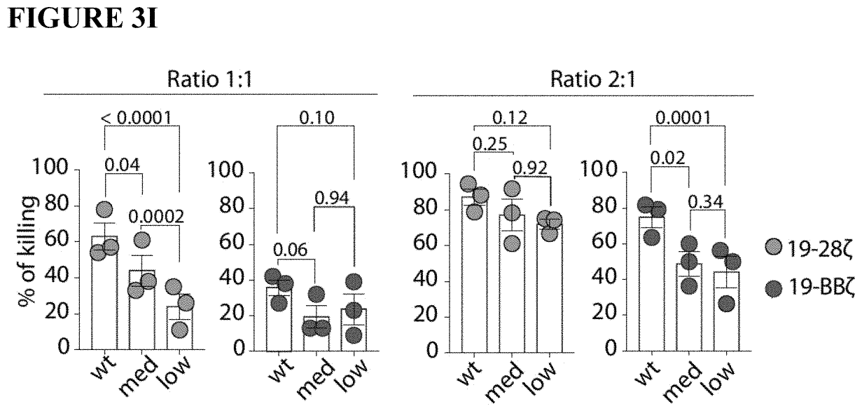

Single-cell cytotoxicity assay: For single cell CTL assays in micro-wells, chamber slides containing a micro-well grid (50×50×50 um/well; Polydimethylsiloxane PDMS) were prepared and submerged in 10% FBS/RPMI-1640 media without phenol red²³. Bulk 1×10⁴ CAR⁺ T cells already loaded with tracer dye (Cell Tracer Violet; CTV; Invitrogen) were seeded with unlabeled NALM6 cells at a ratio of 1:1 in the presence of 1.5 μM of Propidium Iodide (PI; Life Technologies) to enable visualization of cell death. Images were acquired with an Axio0bserver.Z1 microscope (Carl Zeiss) using 20x/0.5 or 40x/0.6 objectives. Appropriate excitation and emission filters were chosen to image CTV and PI stain and bright field. 15 positions (1 position=36 micro-wells) for each chamber were imaged every 10min for 24 hours. Acquired images were processed using a custom macro written on ImageJ software. Each individual well was scanned for the presence of T cells. These wells were then analyzed to identify wells containing a single target cell and one or two effector T cells (as indicated in FIG. 3 legend) and interactions were recorded. In wells where conjugate formation led to target death (lytic conjugates), duration of interaction between E:T was recorded from the formation (start point, T₀) to the interruption of the conjugate (end point, T_{PI}). Abortive T cell contacts (non lytic conjugates) lasting at least 20 min but not leading up to target cell lysis as reflected by PI positivity within the 24-hour of observation. Interruption of the non-lytic conjugate can be either due to conjugate dissociation or T cell death. Contact events lasting for only 1 frame (10min) were not included. All analyses were performed in a blinded fashion. Expected killing frequencies in wells with two T cells (P-est._{2/1}) were calculated based on the percent killing observed in wells with a single target cell and one T cell (P-obs._{1/1}). The frequency of killing under the hypothesis of independence (i.e., simply additive killing) was calculated using the following formula: P-est._{2/1}=P-obs._{1/1}+[P-obs._{1/1}−(P-obs._{1/1}*P-obs._{1/1})]. Cooperative T cell killing is inferred if P-obs._{2/1}>P-est._{2/1}.

CD19 gene targeting: NALM6^{wt} cells were used to generate NALM6^{med} , NALM6^{low}, and NALM6-CD19^{KO}. The gRNA sequence “ctagtggtgaaggtggaagg” was cloned into a PBS-gRNA-MCSV2 vector. NALM6 WT were transfected by electrotransfer of Cas9 mRNA and PBS-gRNA-CD19-MCSV2 vector using an AgilePulse MAX system (Harvard Apparatus). For electroporation, 3e6 cells were mixed with 5 μg of Cas9 mRNA and 5 μg of PBS-gRNA-MCSV2 coding for CD19-targeted gRNA into a 0.2 cm cuvette. Following electroporation cells were seeded into culture medium and incubated at 37° C., 5% CO₂. KO efficiency for CD19 was assessed by flow cytometry and deep sequencing of the KO site. Purification of edited cells was performed by FACS sorting. NALM6^{med} cell line was repeatedly sorted. NALM6^{low} were subcloned using single cell sorting.

Deep sequencing: DNA was extracted using DNeasy Blood&Tissue kit (Qiagen) according to the manufacturer's protocol. To confirm and track editing events in CD19 locus, the region surrounding the site of interest was amplified using the primers (F: gaggctcagagagggtaag (SEQ ID NO: 149) and R: gtgccccggagagtctg (SEQ ID NO: 150) Following end repair and A-tailing, standard Illumina-compatible forked adapters (IDT) were ligated to the amplicon using the Kapa Hyper Library Preparation kit (catalog # KK8504). After library preparation, the amplicons were sequenced on a Hiseq 4000, PE125 to a depth of ˜100,000-1 million reads. Sequencing data was analyzed using the Crispresso pipeline and amplicons were automatically counted after trimming using a custom R program script.

Confocal microscopy: CAR T cells expression a CAR-GFP fusion and NALM6 cells were seeded at 1:1 ratio onto poly-L-lysine coated glass surface chamber slides. Cells were then co-cultured at 37° C. for 1 hour. Cells were fixed by adding of 4% paraformaldehyde into the culture medium (final concentration 1%) and incubating for 15 minutes. After fixation cells were washed twice with PBS. Cells were stained using automated system Leica Bond RX Protocol F. Monoclonal mouse anti-CD19 (clone BT51E, Leica Microsystems), chicken polyclonal anti-GFP (Abcam) and rabbit polyclonal anti-CD3 (Dako, Tyramide Alexa Fluor488 (Life Technologies), with Tyramide Alexa Fluor594 (Life Technologies), and AffiniPure Fab fragment rabbit anti-goat IgG-Alexa Fluor 647 (Jackson Immunoreserach) were used. For nucleus staining, cells were incubated in 5 μg/ml DAPI/PBS solution for 5 min. Slides were mounted using Mowiol® fluorescence mounting media (Mowiol® 4-88 Reagent-Calbiochem, Darmstadt, Germany) prepared in glycerol and Tris-HCl buffer according to the vendor protocol. Cells were kept in the dark at −20° C. For imaging, confocal z-stacks were taken at optimal imaging parameters with a LSM 880 Confocal Microscope with Airyscan with a 63x 1.4 NA Oil Immersion Objective (Carl Zeiss Microimaging). ImageJ software was used to generate the figures.

Transcriptome analysis: Cells were sorted into Trizol LS (Invitrogen) and then submitted to the Integrated Genomics Operation at MSKCC for RNA extraction. After ribogreen quantification and quality control on bioAnalyser, 500ng of total RNA underwent library preparation using the Truseq Stranded Total RNA library preparation chemistry (Illumina), with 6 cycles of PCR. Samples were barcoded and run on a Hiseq 2500 1T in a 50bp/50bp Paired end run, using the TruSeq SBS Kit v3 (Illumina). An average of 51 million paired reads were generated per sample and the percent of mRNA bases was 58% on average. The output FASTQ data files are mapped to the target genome using the rnaStar aligner which maps reads genomically and resolves reads across splice junctions. The 2 pass mapping method outlined was used, in which the reads are mapped twice. The first mapping pass uses a list of known annotated junctions from Ensemble. Novel junctions found in the first pass are then added to the known junctions and a second mapping pass is done (on the second pass the RemoveNoncanoncial flag is used). After mapping, the output SAM files were post processed using the PICARD tools to: add read groups, AddOrReplaceReadGroups which in additional sorts the file and coverts it to the compressed BAM format. the expression count matrix was computed from the mapped reads using HTSeq (www-huber.embl.de/users/anders/HTSeq) and one of several possible gene model databases. The raw count matrix generated by HTSeq are then be processed using the R/Bioconductor package DESeq (www-huber.embl.de/users/anders/DESeq) which is used to both normalize the full dataset and analyze differential expression between sample groups.

Quantitative mass spectrometry: Cells were lysed (8M Urea in 200mM EPPS pH 8.4 lysis buffer containing Roche protease inhibitor cocktail), sonicated (1 min), centrifuged (8000×g for 10 min at 4° C.) then the supernatant transferred to new tubes and protein concentration determined by BCA (Pierce). Lysates were reduced with DTT (37° C., lhr), alkylated with iodoacetamide (RT, 30 min, dark), and quenched with an additional 5mM DTT (RT, 15 min, dark). In-solution Lys-C digestion (E: S 1:100) was performed at 37° C. for 6 hours, then the urea concentration diluted to 1.3 M with buffer and trypsin was added (1:100) and further incubated another 16hr at 37° C. Enzyme activity quenched with TFA and samples frozen (−80C). Based on the BCA assay, 10 ug aliquots were removed from each sample for fractionation.

To fractionate the mixture of peptides, a 5-cutter method based on the StageTip technique of Rappsibler using C18 disk (3M Empore Solid Phase Extraction Disk, #2315) was used. Prior to loading samples, StageTips were conditioned with 504, washes of acetonitrile, 50% acetonitrile in 2.5 mM ammonium bicarbonate, then 2.5mM ammonium bicarbonate. After washing, fractions 1-5 were eluted with 30uL of 8, 15, 22, 30, and 50% acetonitrile in 2.5mM ammonium bicarbonate. Fractions were frozen, lyophilized to dryness, and reconstituted in 54, of 0.1% formic acid for LC-MS/MS analysis.

Fractions were analyzed by Microcapillary LC coupled (Waters NanoAcquity, 100-₁1m i.d.×10 cm C18 column, 1.7 um BEH130, configured with a trap column) to a Q-Exactive Plus mass spectrometer (Thermo Fisher Scientific). Peptides were eluted at 300 nL/min using a 4hr acetonitrile gradient (2-50% acetonitrile in water with 0.1% formic acid). The QE Plus was operated in automatic, data-dependent MS/MS mode with one MS full scan (380-1800 m/z) at 70,000 mass resolution and up to ten MS/MS scans at 17,500 resolution, an isolation window of 1.5 amu and normalized collision energy of 27. AGC was set to 3×10⁶for MS1 and 5×10⁴ and 60ms max IT for MS2.

MS data was processed using MaxQuant version 1.5.5.30 and the Uniprot human protein sequence database (downloaded Aug. 1, 2017). Carbamidomethylation of C was set as a fixed modification and the following variable modifications allowed: oxidation (M), N-terminal protein acetylation, deamidation (N and Q), phosphorylation (S,T,Y). Search parameters specified an MS tolerance of 8ppm, an MS/MS tolerance at 40 ppm and full trypsin digestion, allowing for up to two missed cleavages. Peptides were required to be at least 7 amino acids in length with 1% false discovery rates (FDRs) calculated at peptides level.

Statistics: All experimental data are presented as mean±s.e.m. No statistical methods were used to predetermine sample size. Appropriate statistical tests were used to analyze data, as described in figure legend. Statistical analysis was performed on GraphPad Prism 7 software. Two-sample binomial tests for proportions (one-sided) were used to evaluate: H0: P-obs_{2/1}<=P-est_{2/1} vs. H1: P-obs_{2/1}>P-est_{2/1}. The experiments were considered as a whole and did not account for any batch or donor effect given the limited sample size. Evaluating the percent killing within each experimental donor could lead to inconsistent conclusions across donors and more importantly an inflated type 1 error rate. It was noted that the approach is more conservative by not accounting for the correlation within a donor and would likely result in increased power if it was accounted for. A less stringent type 1 error rate of 10% was used on the aggregate data (significance as p<0.1). No cooperative effect (additive effect) was defined to be the null hypothesis, i.e. H0: P-obs2/1<=P-est_{2/1}, which assumes T killing events are independent in wells containing one target cells and two effector T cells.

### Results

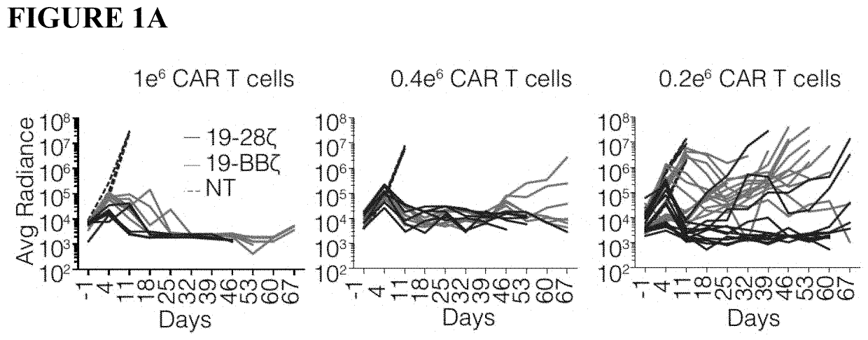

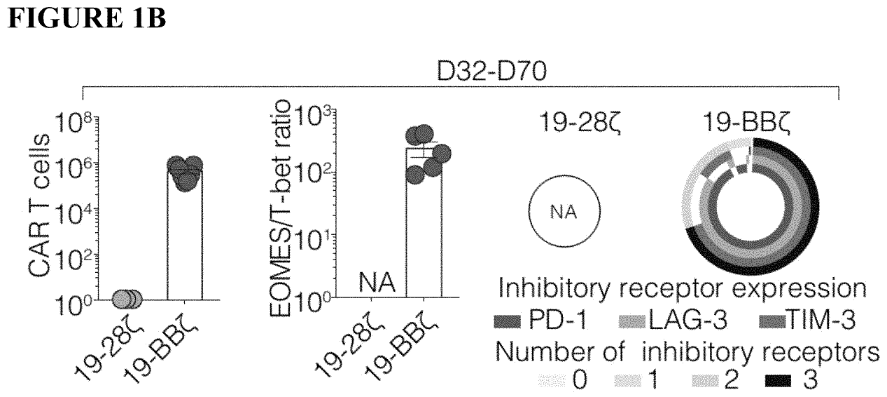

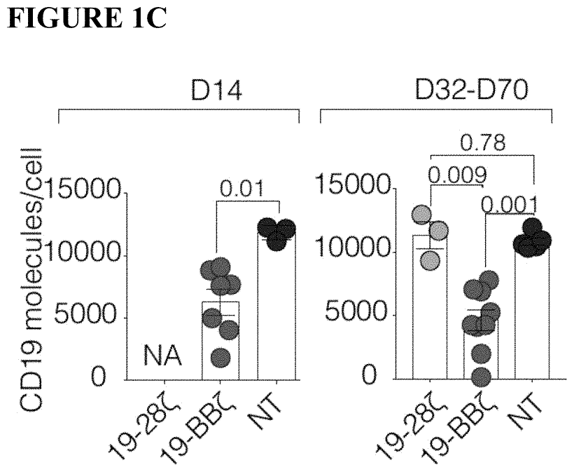

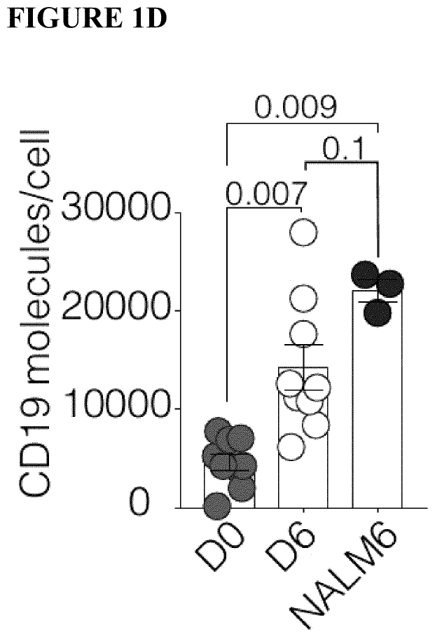

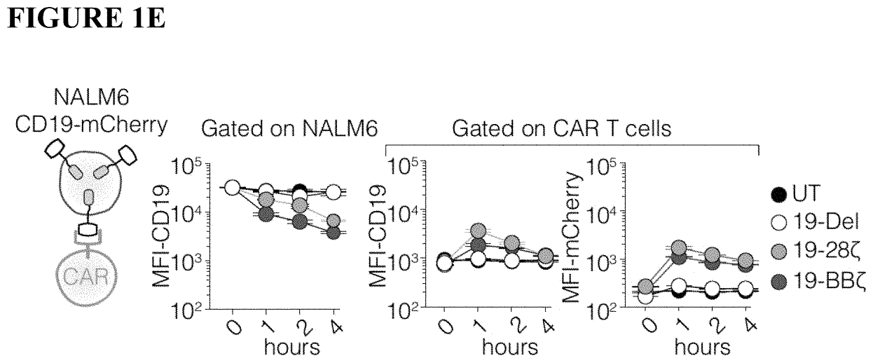

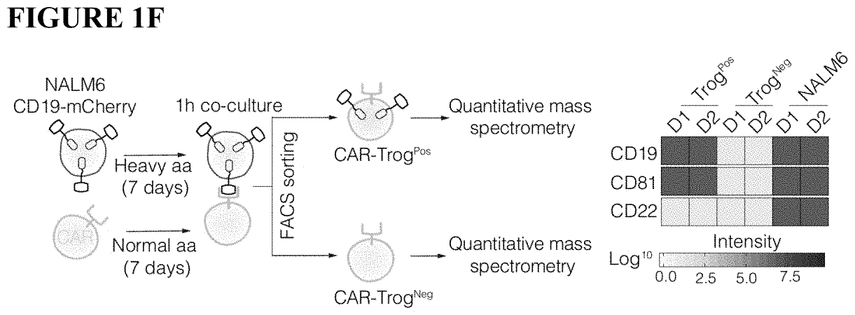

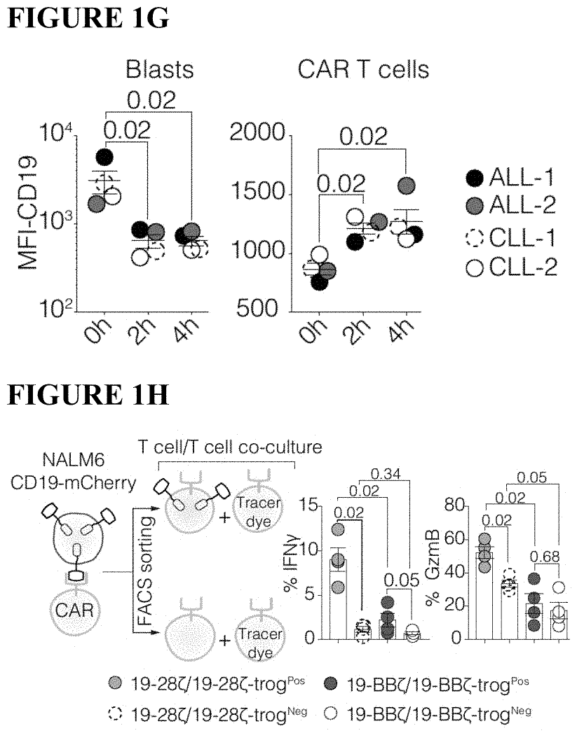

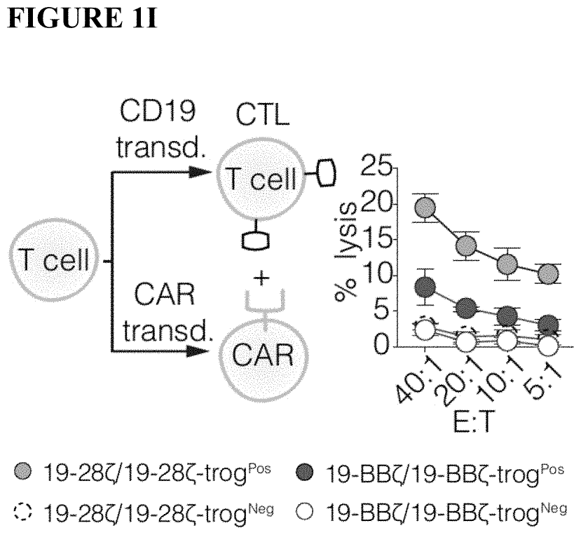

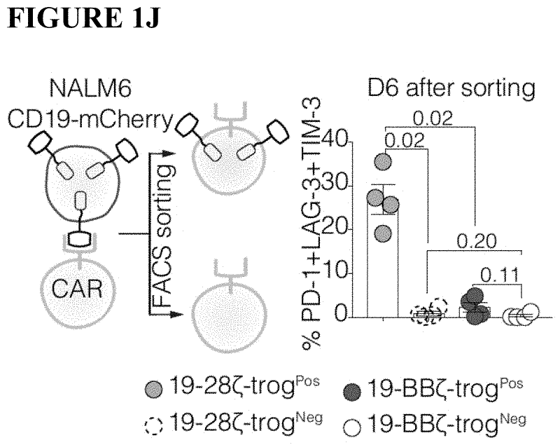

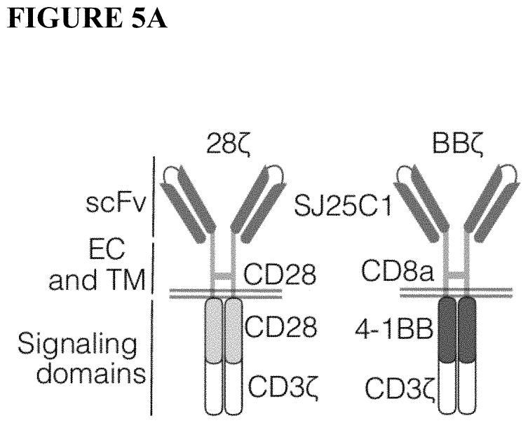

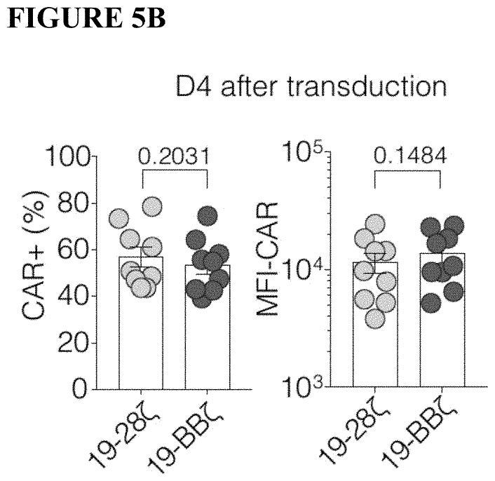

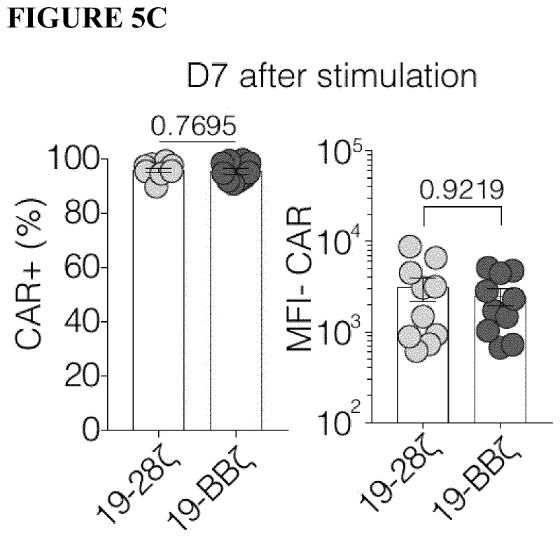

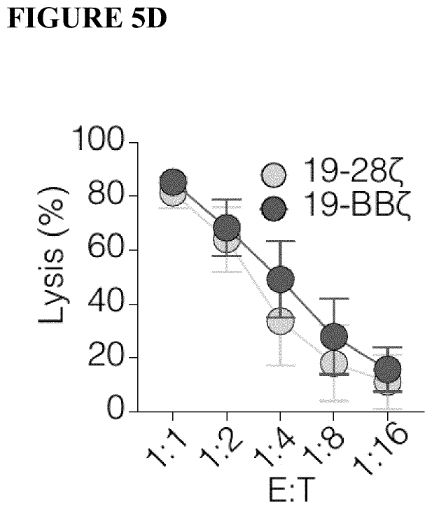

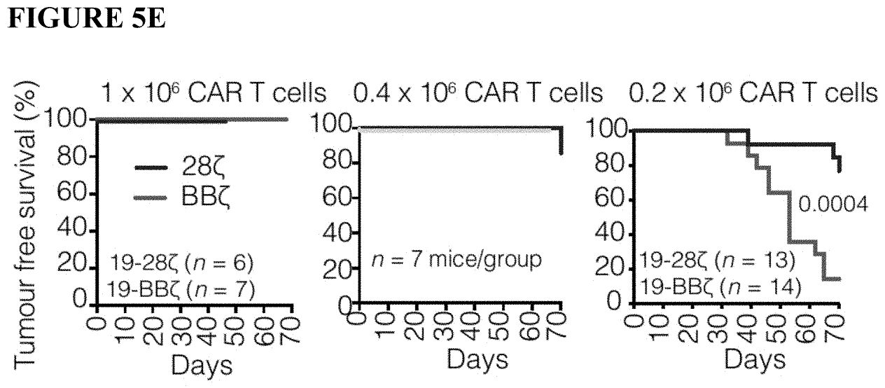

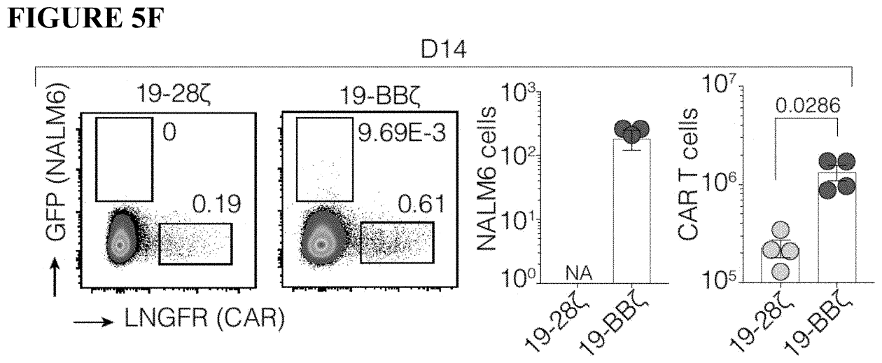

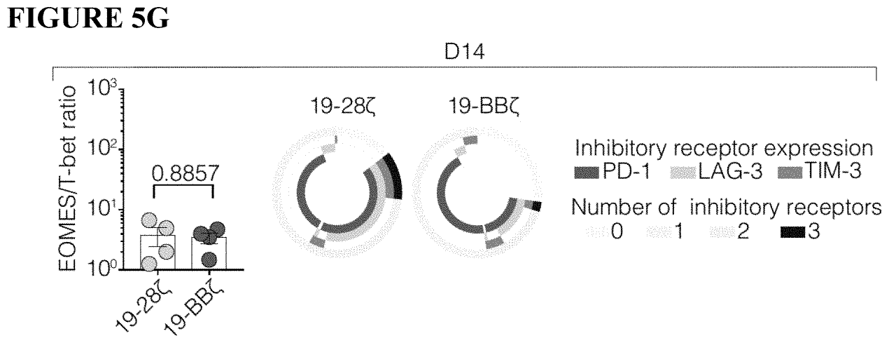

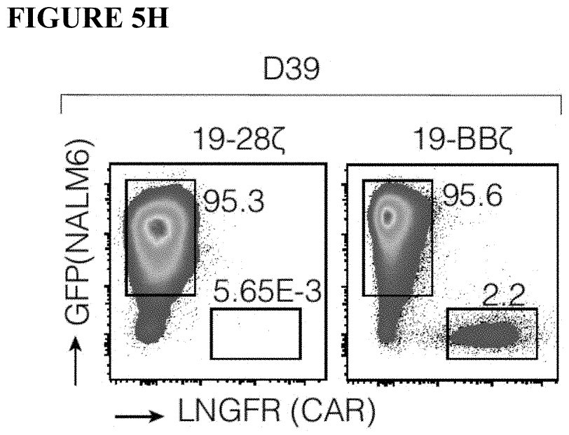

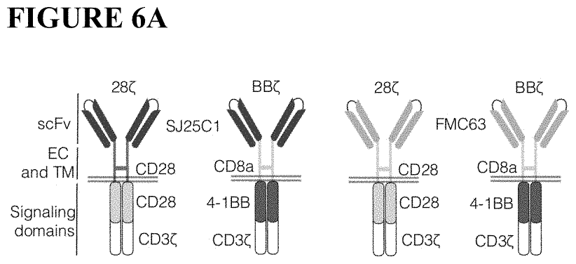

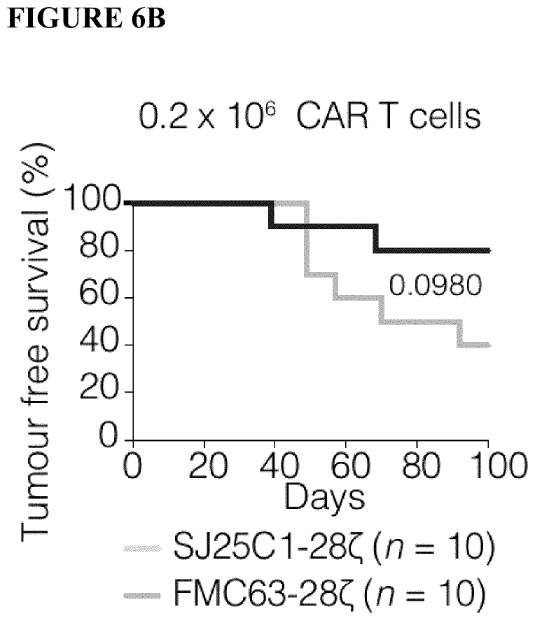

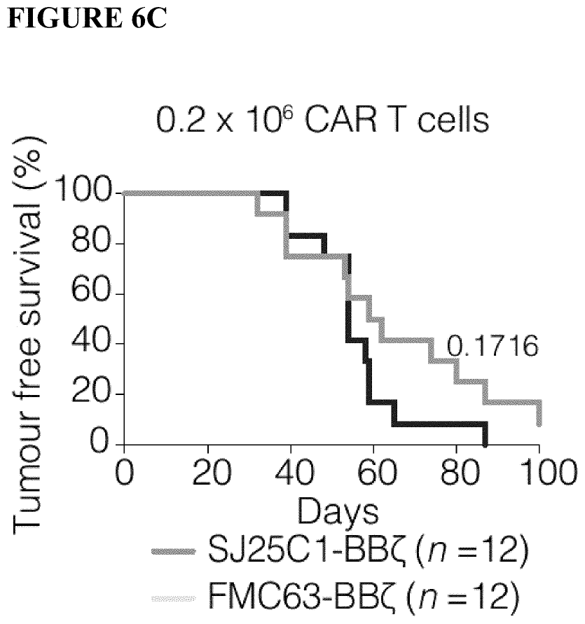

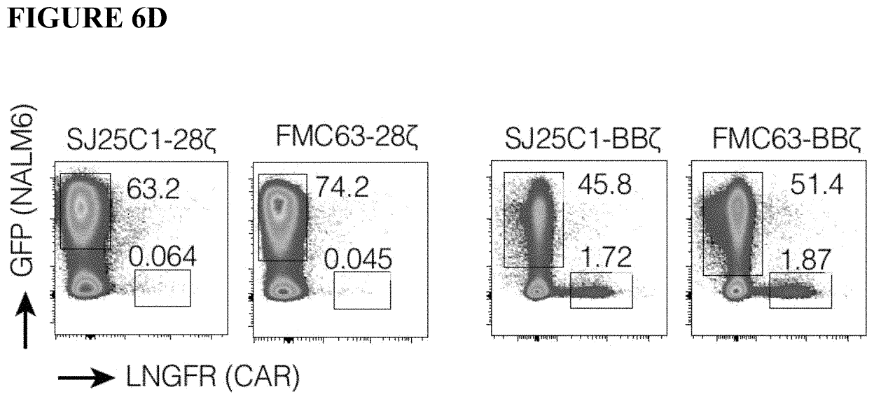

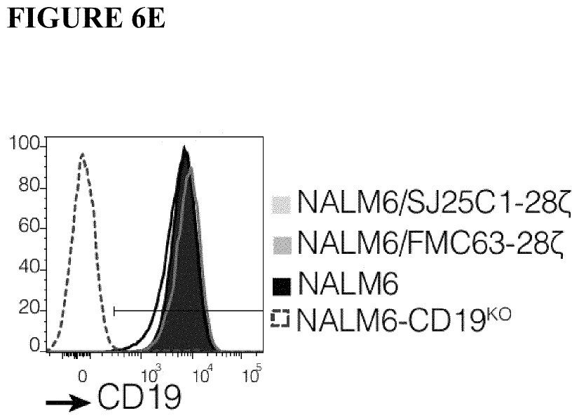

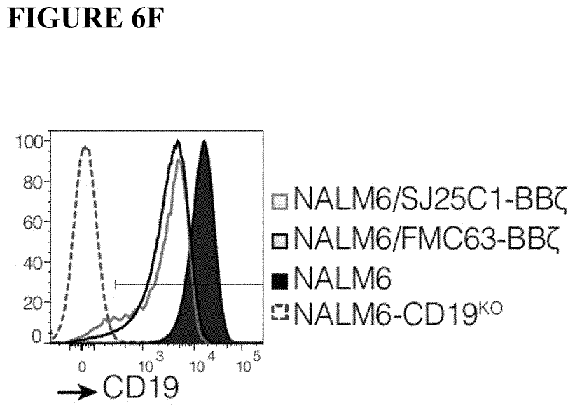

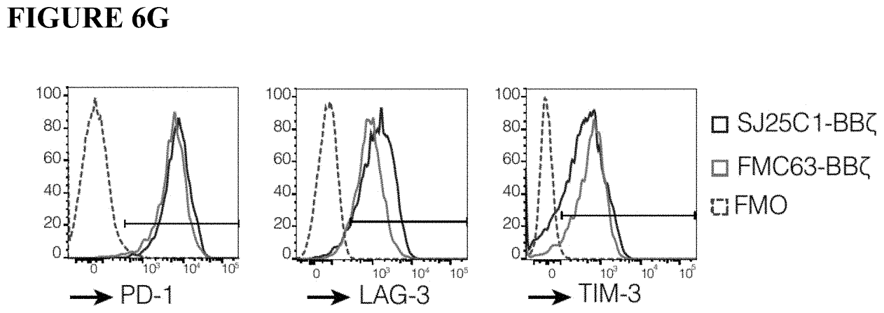

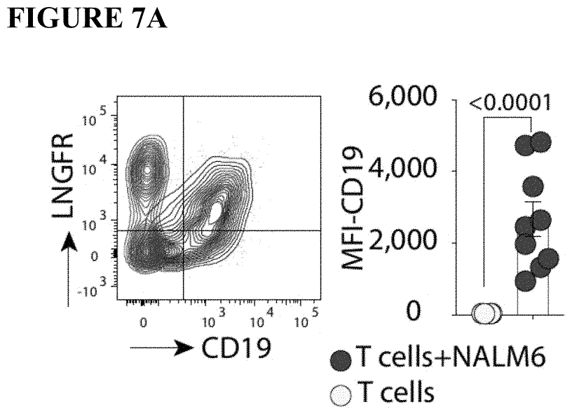

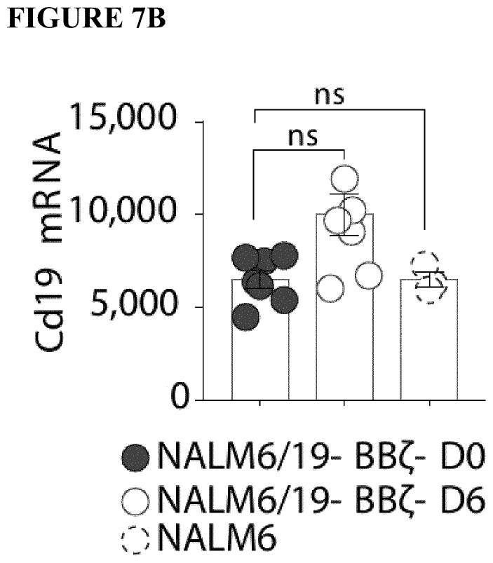

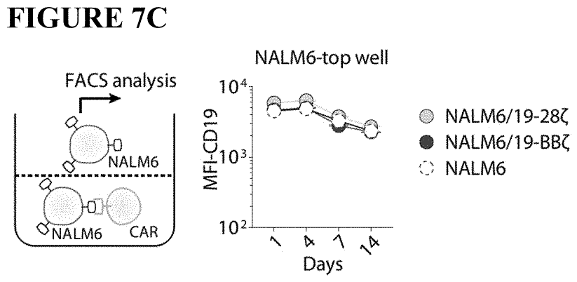

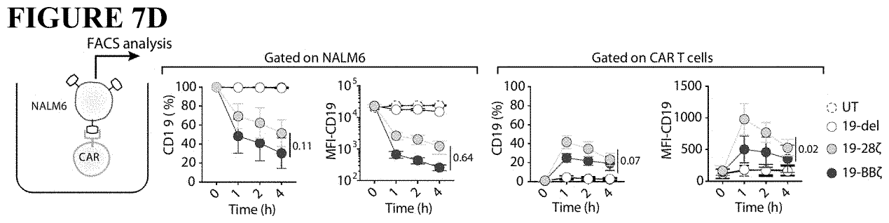

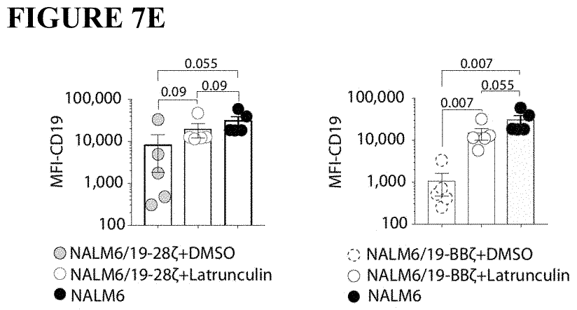

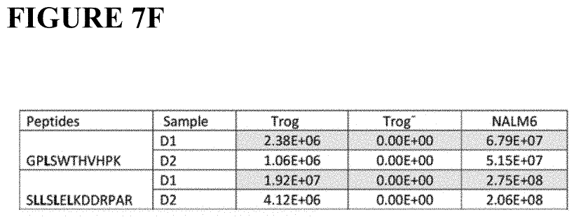

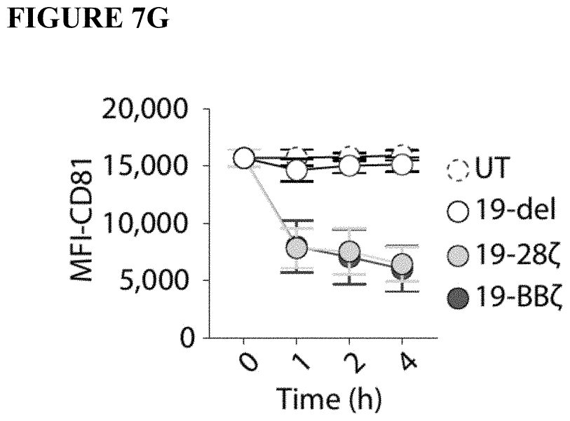

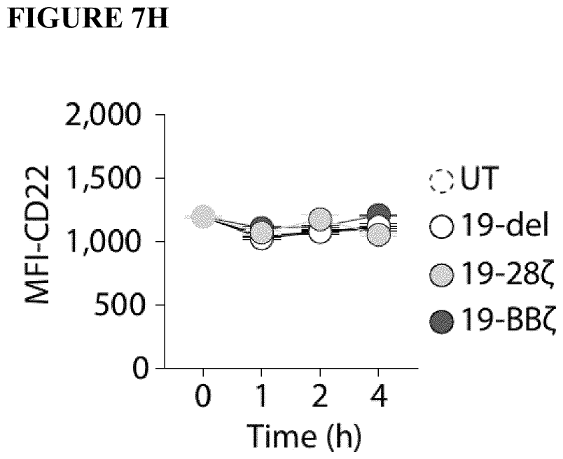

CAR therapy relapse was modelled by infusing limiting doses of CD19 CART cells in the well-established NALM6 leukaemia model¹⁰⁻¹⁶(FIG. 5A). CARs encompassing CD28 or 4-1BB costimulatory domains (referred to as 19-28ζ and 19-BBζ) effectively controlled NALM6^{wt} leukaemia at the dose of 0.4-1×10⁶ CART cells but allowed for frequent leukaemia relapse at the dose of 0.2e⁶ (FIG. 1A, FIG. 5B-5E). Whereas both CART cells showed limited evidence of exhaustion two weeks after infusion (FIG. 5F-5G), 19-BBζ cells were markedly exhausted by the time of relapse, whereas 19-28ζ cells were no longer detected (FIG. 1B, FIG. 5H), consistent with clinical experience¹³⁻¹⁶ and CAR stress test models¹². CD19 expression was reduced in progressing 19-BBζ-treated NALM6^{wt}, averaging 4,500 molecules per cell, down from the starting ˜11,000, which were found in 19-28ζ relapses and in untreated mice (FIG. 1C). Loss of CD19 occurred early on, being already present by day 14 and thus occurring in the presence of abounding CAR T cells (FIGS. 1B-1C and FIG. 5F). The same patterns were found with CD19 CARs utilizing either SJ25C1¹⁶ or FMC63¹³⁻¹⁵ scFv's (FIG. 6). Concurrent with decreased CD19 expression in tumour cells, a large fraction of CAR T cells stained positive for CD19 (FIG. 7A). Strikingly, CD19 expression in the retrieved NALM6^{wt} cells was reversible upon short-term culture (FIG. 1D). Given the little variation in CD19 mRNA levels (FIG. 7B), these findings indicated that a reversible, post-transcriptional loss of CD19 occurred in the presence of CART cells. CD19 expression did not vary when fresh NALM6^{wt} were segregated from CAR T cells in trans-wells, but promptly decreased when T cells were co-cultured (FIG. 7C AND 7D). CD19 was not lost in co-cultures with untransduced T cells or T cells expressing a non-signalling CD19 CAR (FIG. 7D). The transfer of CD19 protein from NALM6^{wt} to T cells thus displayed the hallmarks of CAR-mediated trogocytosis, as further compounded by inhibition by actin polymerization blockers¹⁷(FIG. 7E). Co-culture with CD19^{KO} NALM6 cells expressing a CD19-mCherry fusion molecule resulted in detection of both mCherry and CD19 in T cells, demonstrating whole-protein CD19 membrane extraction (FIG. 1E). Loading of CD19-mCherry NALM6 cells with heavy amino-acids and 19-28ζ cells with light amino-acids and then sorting mCherry-positive (Trog^{Pos}) and -negative (Trog^{Neg}) singlet T cells after brief co-culture unequivocally demonstrated CD19 peptides in the Trog^{Pos} but not the Trog^{Neg} fraction (FIG. 1F, FIG. 7F). CD81, which is complexed with CD19, was also detected in Trog^{Pos} but not Trog^{Neg} T cells (FIG. 1F), while concomitantly lost in the co-cultivated NALM6 (FIG. 7G). In contrast, CD22 remained unchanged in NALM6 cells and was not detected by mass spectrometry in the T cells (FIG. 1F, FIG. 7H-I).

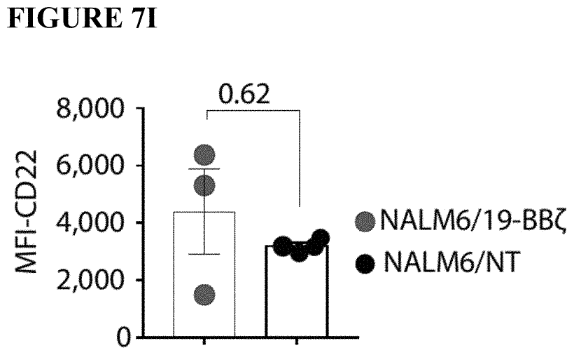

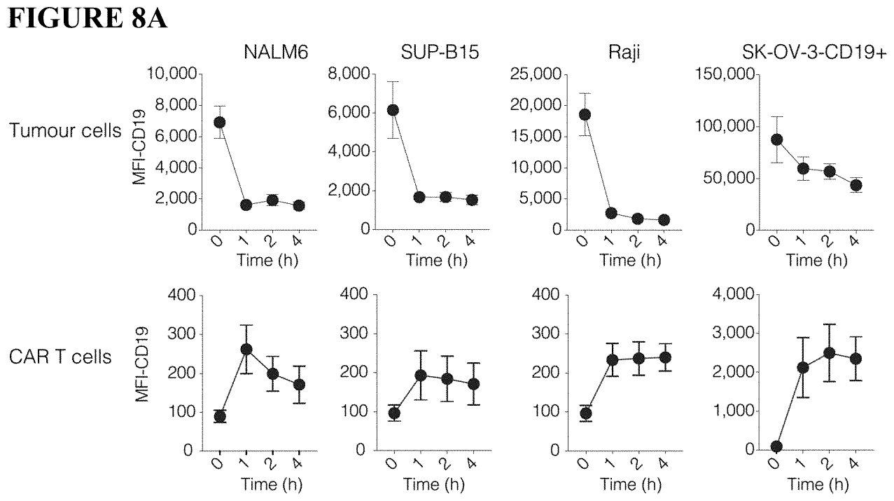

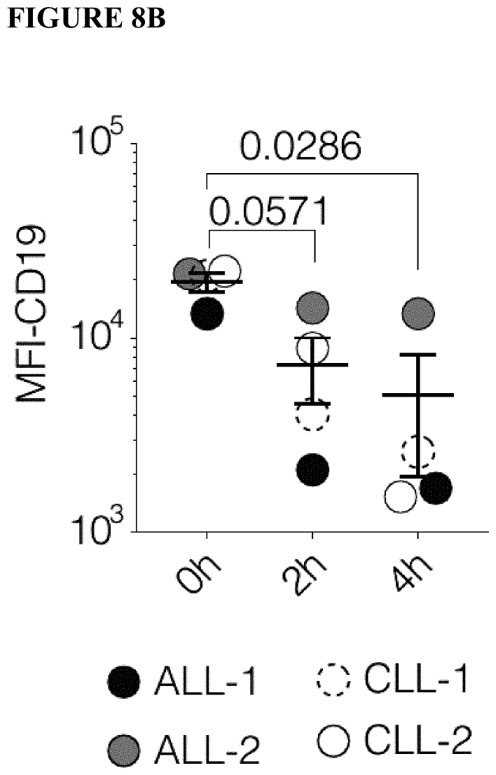

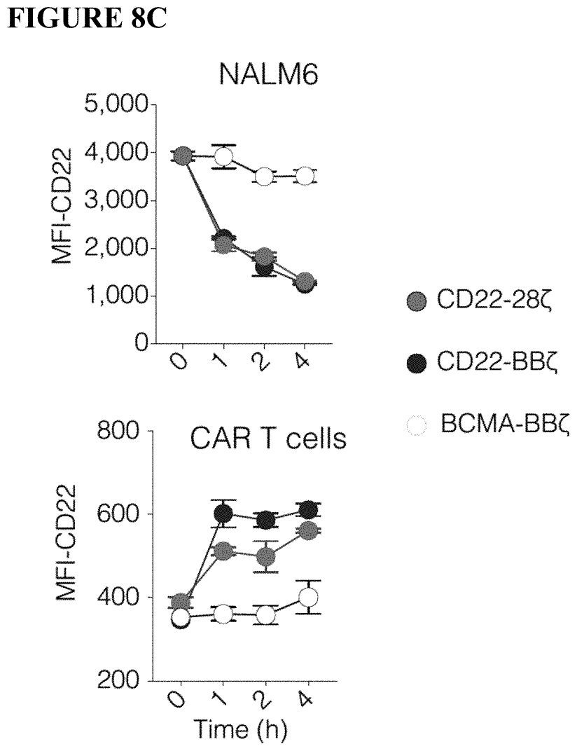

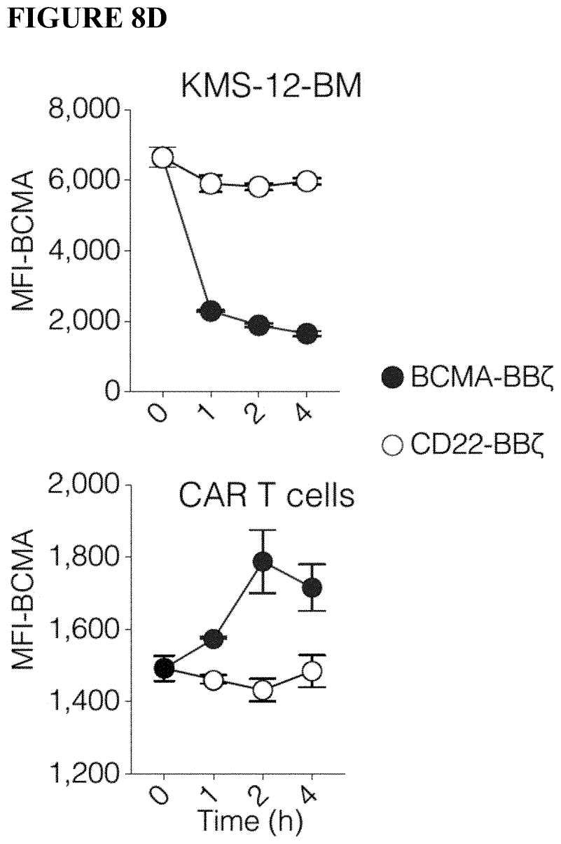

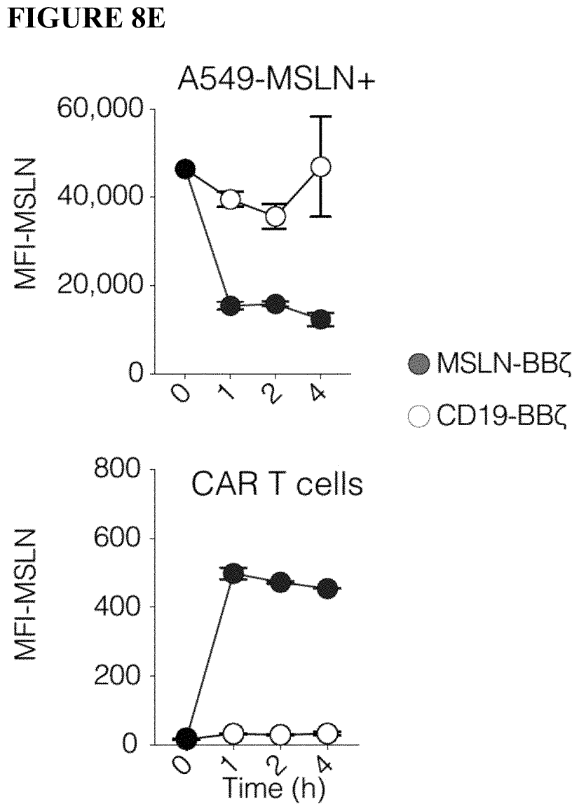

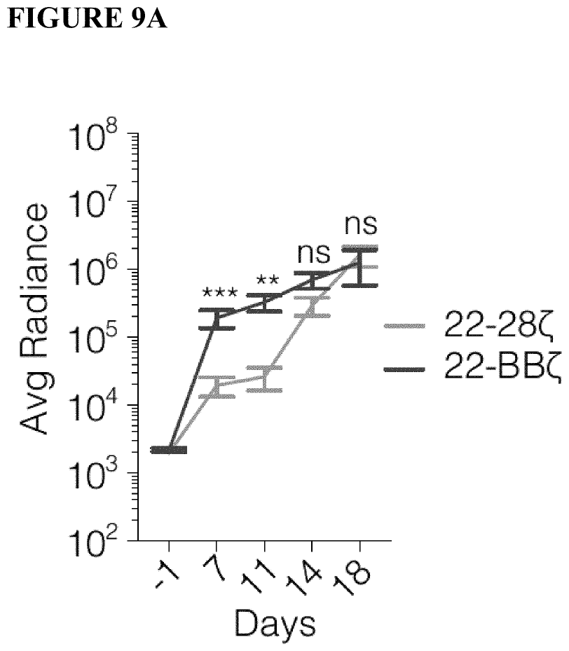

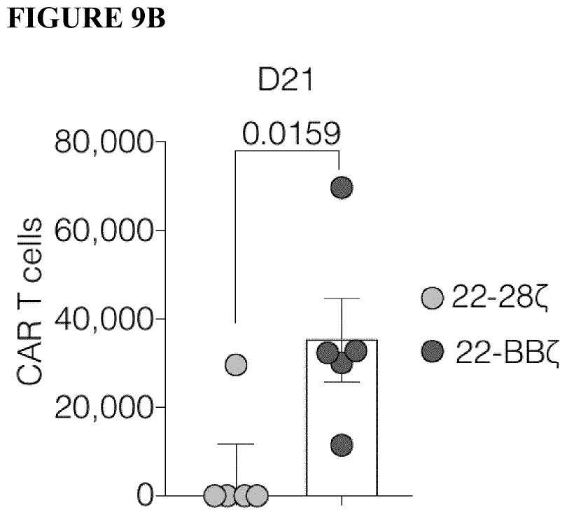

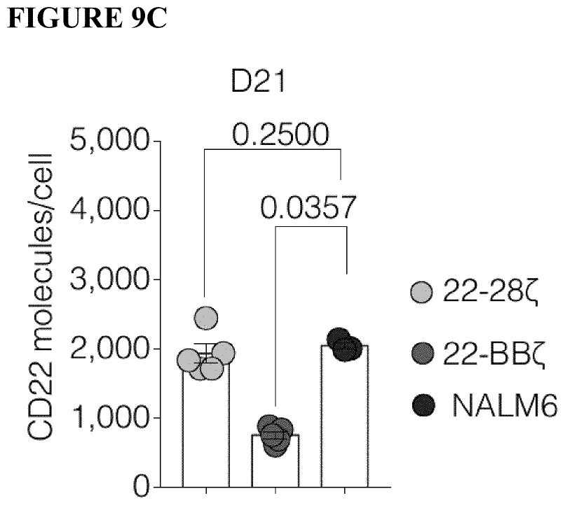

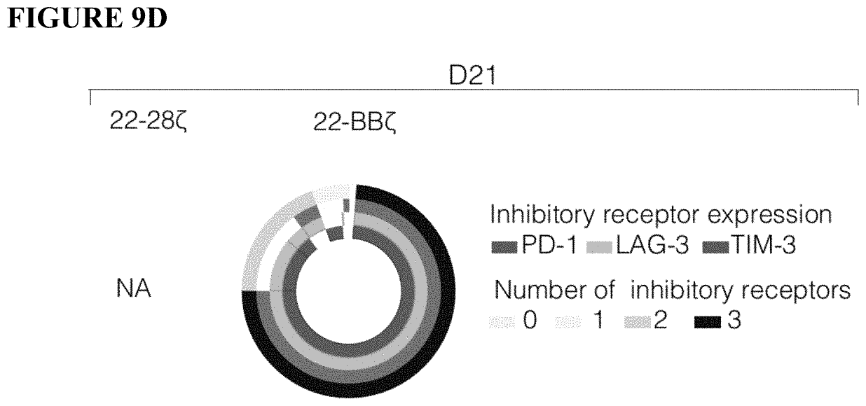

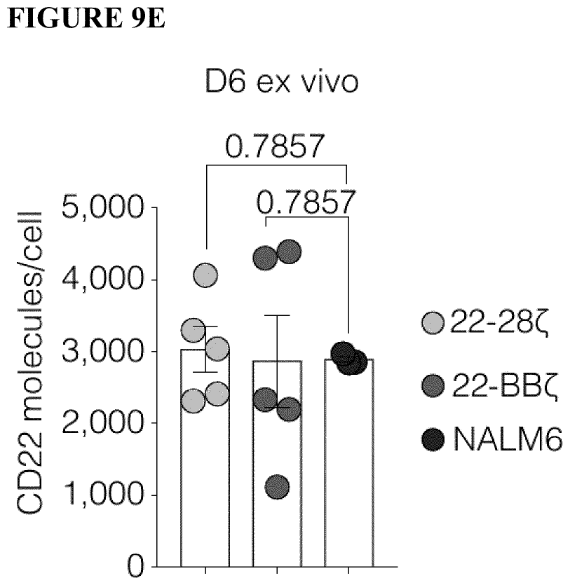

CD19 trogocytosis occurred likewise in co-culture with NALM6^{wt}, SUP-B15, Raji and CD19+ SKOV-3 cells (FIG. 8A) and, importantly, upon co-incubation of primary ALL and CLL patient samples with autologous 19-28ζ cells (FIG. 1g, FIG. 8B). CAR-induced trogocytosis was observed with all other tested CAR targets, including CD22, B-cell maturation antigen (BCMA) and mesothelin (FIG. 8C-E). The same antigen escape profile was achieved upon targeting CD22 in vivo. NALM6^{wt} cells, which express 2000 CD22 molecules per cell at baseline, relapsed with expression of ˜750 CD22 molecules following treatment with 0.2×10⁶ 22-BBζ cells (FIG. 9). Trogocytic target acquisition is thus a general feature of CART cells, likely applying to many if not all antigens.

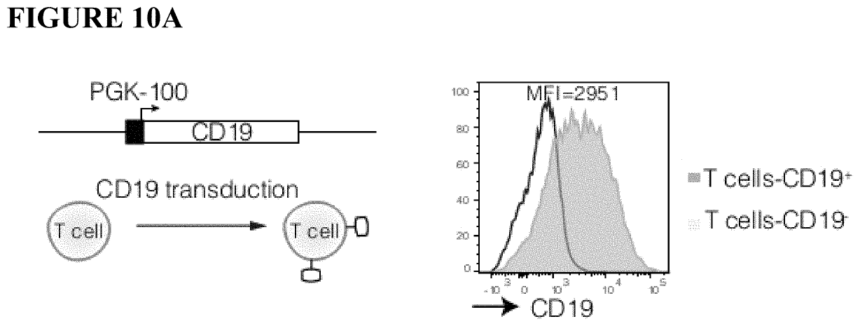

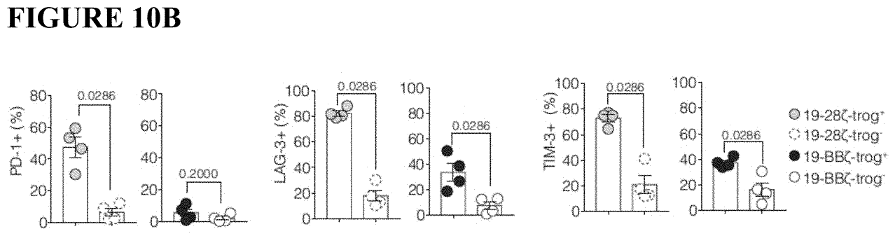

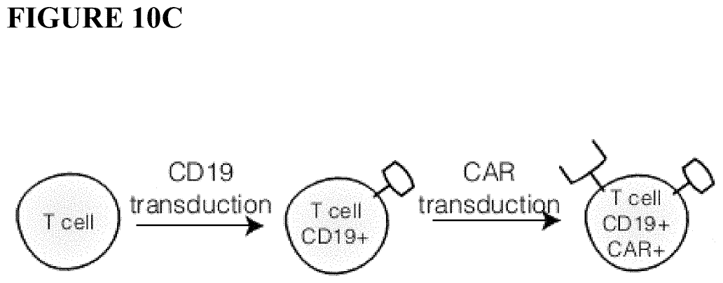

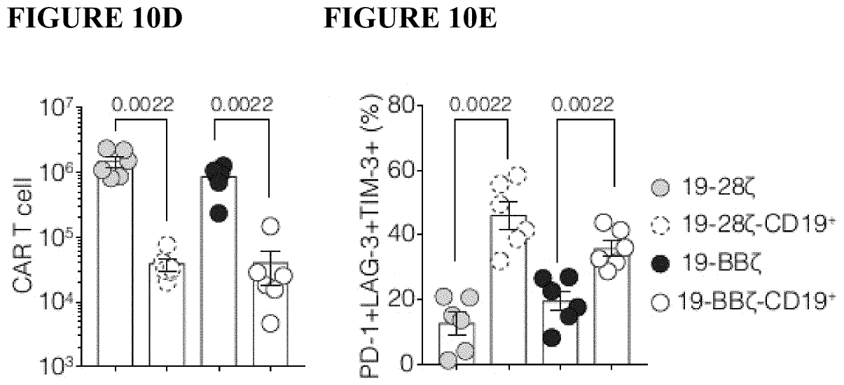

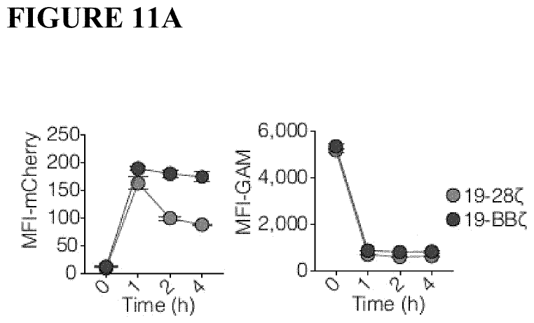

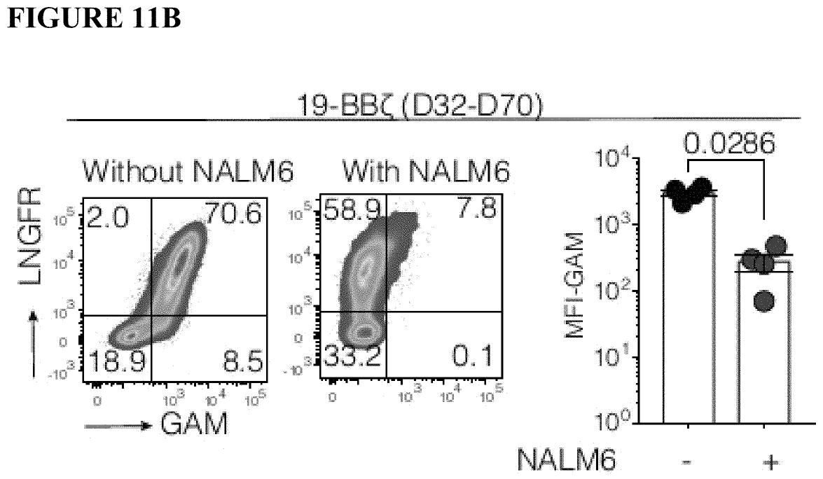

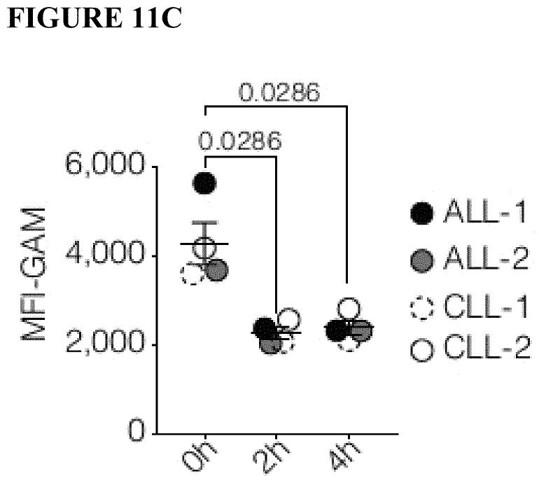

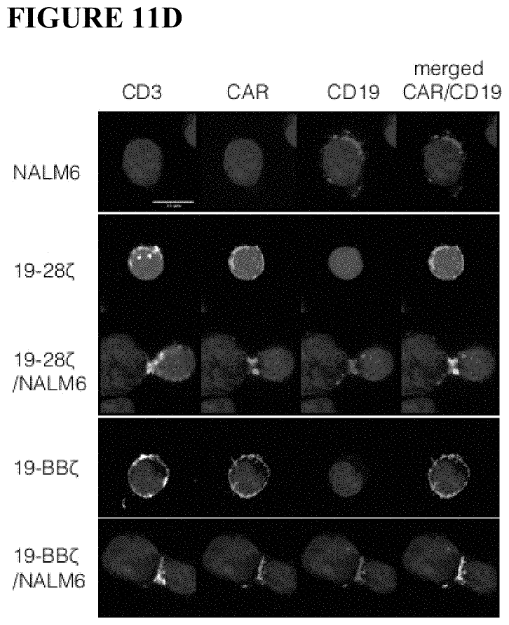

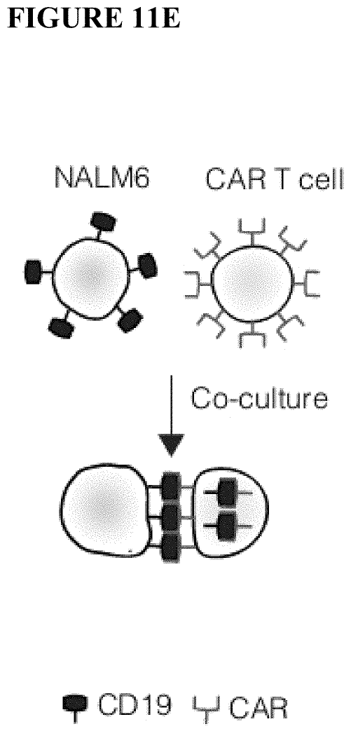

Co-culture of sorted Trog^{Pos} but not Trog^{Neg} CAR T cells with fresh 19-28ζ cells elicited the latter's production of IFNγ and GzmB (FIG. 1H). When stably expressing CD19 at levels approximating those detected after trogocytosis (FIG. 10A), both 19-28ζ and 19-BBζ cells engaged in CD19+ T cell killing, more so the former, consistent with their greater effector function^{12,18} (FIG. 1I). Culture of sorted Trog^{Pos} and Trog^{Neg} T cells for 6 days revealed increasing expression of PD-1, LAG-3 and TIM-3 in the former (FIG. 1J, FIG. 10B). In T cells stably co-expressing CAR and CD19, those that did not succumb to fratricide killing tended to acquire exhaustion markers (FIG. 10C-E). CD19 trogocytosis was associated with diminished cell-surface CAR expression in vitro and in vivo and co-localized intracellular CAR and CD19 (FIG. 11).

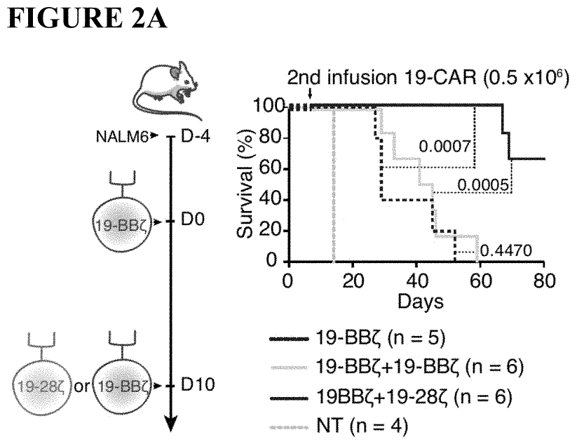

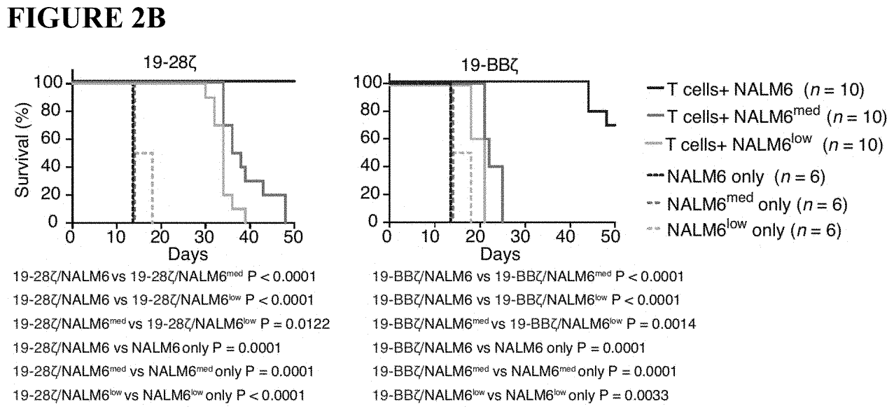

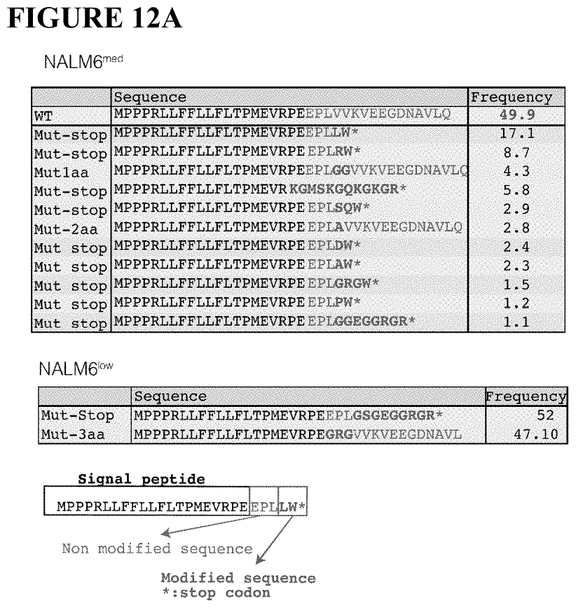

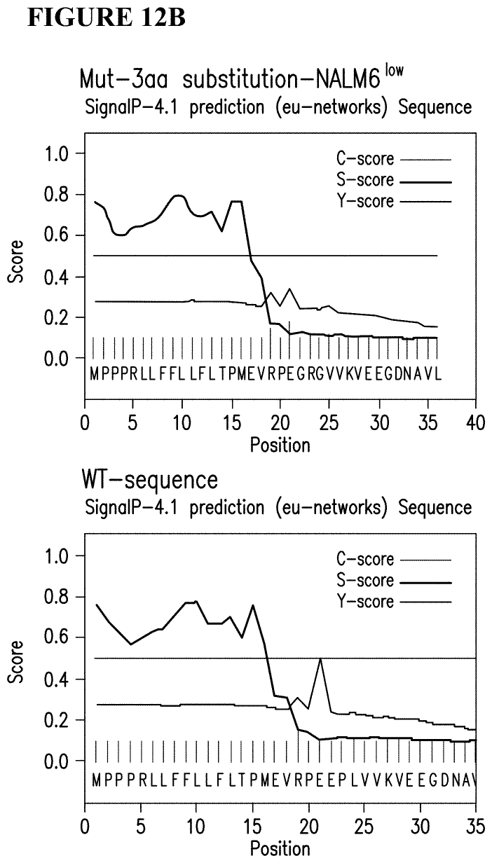

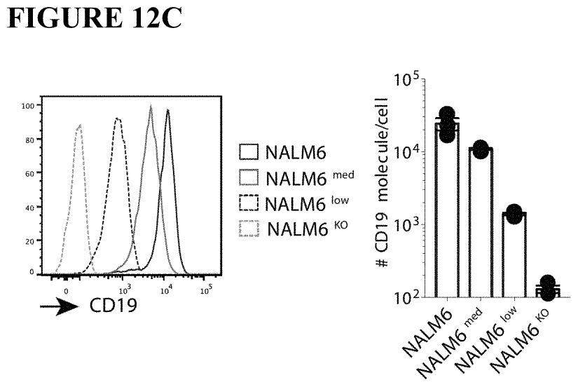

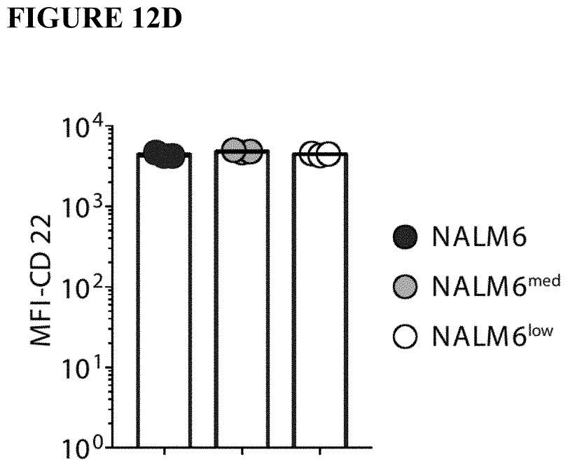

The infusion of “fresh” 19-BBζ T cells 10 days after the initial 19-BBζ treatment failed to rescue the relapse-prone mice (FIG. 2A), suggesting that CD19 density had already decreased to a level eluding 19-BK threshold efficacy. 19-28ζ CART cells however did rescue these relapses (FIG. 2A). Differential antigen sensitivity between the two CARs was confirmed in models utilizing stable, graded CD19 levels (FIG. 2B, FIG. 12). Mice bearing NALM6^{med} or NALM6^{low} tumours showed diminished responses to low-dose CAR therapy, with 19-28ζ cells consistently inducing longer survival than 19-BBζ counterparts (FIG. 2B). These studies thus confirmed that diminution of target antigen density alone can foster CAR T cell resistance. Together with the promotion of T cell exhaustion (FIG. 1J, FIG. 10B-E) and fractricide killing (FIG. 1H-I and FIG. 10D), CAR target extraction is thus poised to promote antigen-low tumour relapse.

Importantly, 19-BBζ cells were able to control NALM6^{wt} when administered at a higher dose (FIG. 1A). It was hypothesized that higher effector:target ratios may overcome clonal sensitivity thresholds. In microwells containing one CAR T cell and one NALM6^{wt} (1:1 E:T, FIG. 3A), 19-28ζ cells were more likely to lyse NALM6^{wt} than 19-BBζ cells (57% vs 39% over 24hrs, FIG. 3B). The time to tumour cell death following the formation of a T cell/NALM6^{wt} conjugate showed that 19-28ζ cells killed NALM6^{wt} after 261±18 min (±SEM) whereas 19-BBζ cells did so in 569±35 min (FIG. 3B). The lesser killing of 19-BBζ cells did not result from poorer antigen recognition as the frequency of non-lytic stable conjugate formation was not lower than found with 19-28ζ cells and the time spent in a non-lytic conjugate higher (FIG. 3C).

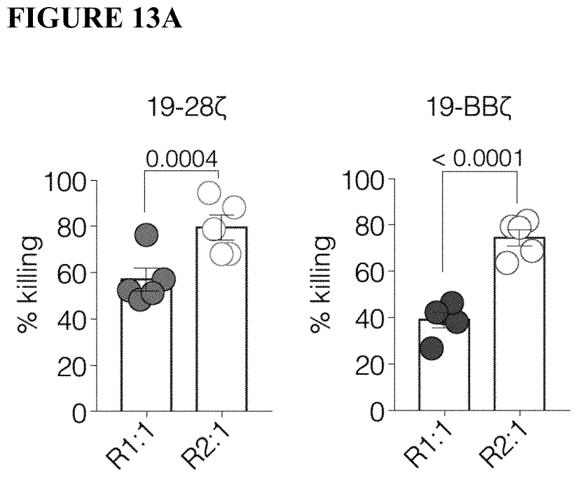

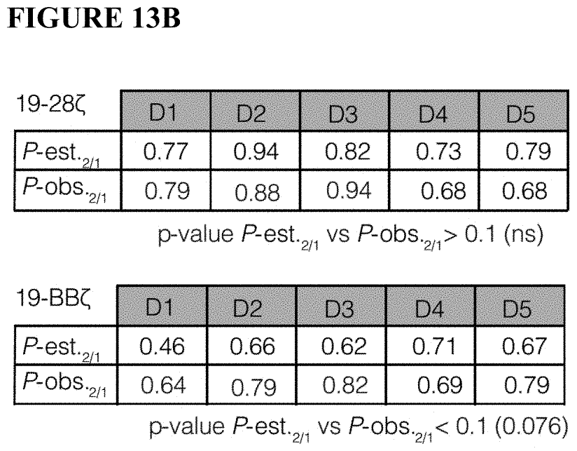

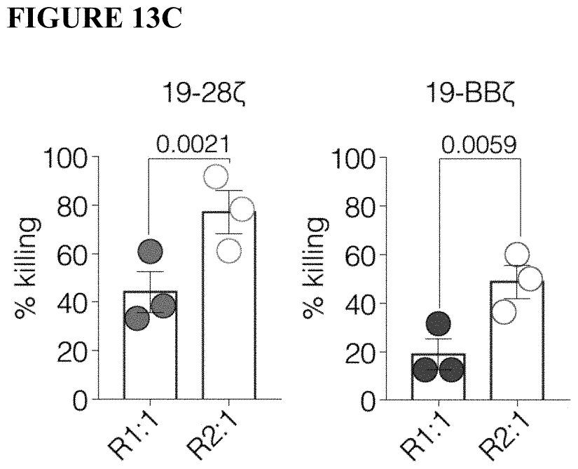

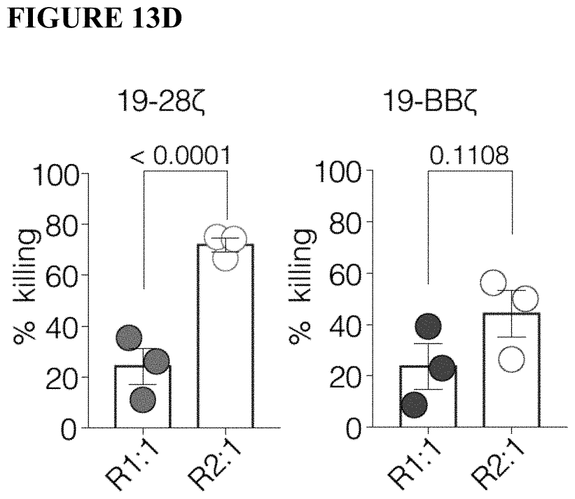

In microwells containing two CAR T cells and one tumour cell (2:1 E:T, FIG. 3D), the frequency of tumour lysis increased to about 75% for both 19-28ζ and 19-BBζ cells, (FIG. 3E). Whereas this increase could be accounted for by additive killing with 19-28ζ cells, the gain seen with 19-BBζ cells exceeded simple additivity (FIG. 3F, FIG. 13A-B). This greater than expected tumor killing accorded with the observed frequency of conjugates comprising 2 T cells bound to the same tumor cell and the shorter time to target cell death measured for the second T cell conjugate (FIG. 3F-H). Microwell studies utilizing the NALM6^{med} and NALM6^{low} target cells confirmed and extended these results, showing that 19-BBζ cells were more sensitive to antigen loss than 19-28ζ cells and now revealing cooperativity between 19-28ζ cells as CD19 density decreased (FIG. 31, FIG. 13C-D). The possibility of cooperative killing, previously noted in chronic infection models¹⁹, underscores the benefit of achieving higher effector:target ratios and the potential for population-based killing to transcend clonal limitations.

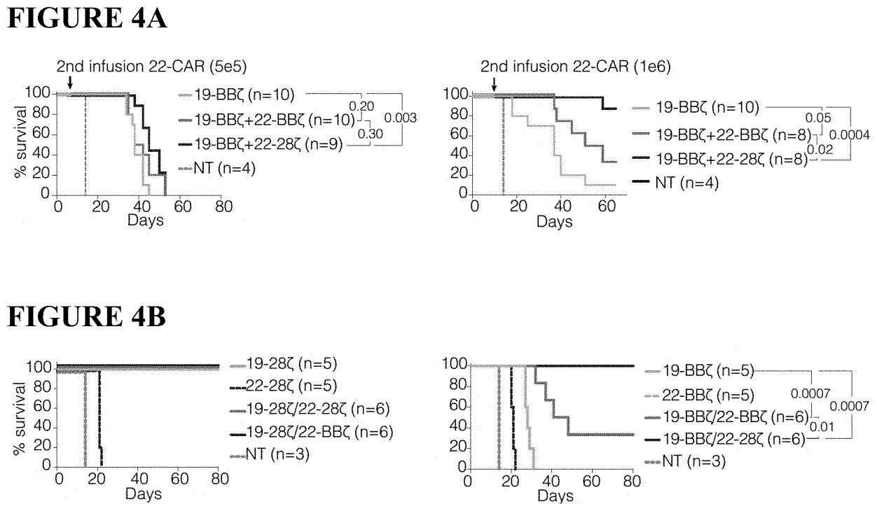

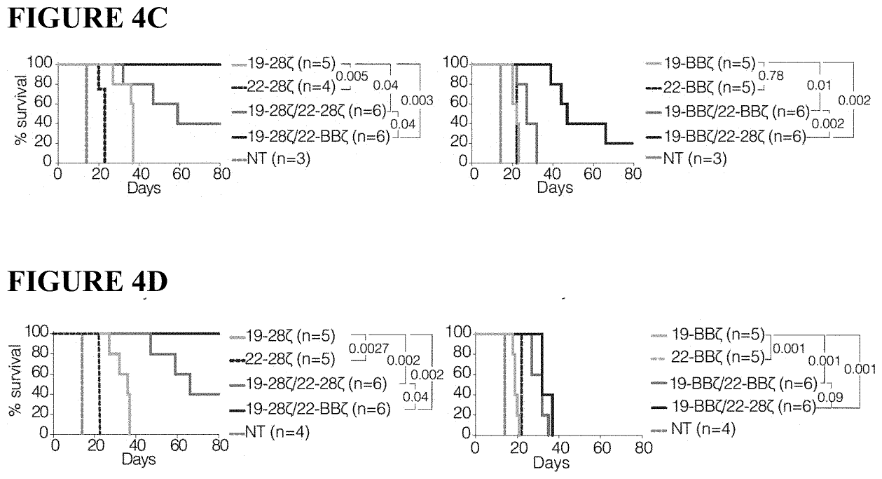

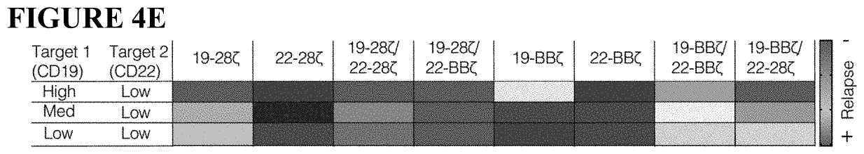

These considerations raised the possibility that combinatorial targeting, which has been proposed as a means to preempt antigen-negative escape^{5,7}, could mitigate antigen-low escape. The inventors thus investigated combinatorial possibilities taking advantage of their calibrated NALM6 lines (FIG. 12D). Treating mice with established NALM6^{wt} with 0.2×10⁶ 19-BBζ cells followed by a second infusion of 0.5×10⁶ 22-28ζ or 22-BBζ cells was ineffective (FIG. 4A), resulting in reduced CD22 expression and poor CAR T cell expansion (FIG. 9B and C). A higher CAR T cell dose (1e⁶) afforded a better survival benefit, more so with 22-28ζ (FIG. 4A). Dual targeting proved more effective in preventing antigen relapse (0.2×10⁶ CD19/CD22 CAR T cells, FIG. 4b) than sequential infusions (FIG. 4A). If CD19 were targeted with a BBζ CAR, targeting CD22 with a 28ζ CAR proved to be much more efficacious (FIG. 4B).

Under lower antigen density conditions, the 19-28ζ/22-BBζ combination afforded the most durable responses (FIG. 4C-D), outperforming BBζ CAR pairings. At the lowest CD19 level, 19-BBζ CAR therapy could no longer be rescued by co-targeting the low-density CD22 target, regardless of which CD22 CAR was used. Thus, combinatorial targeting can reduce escape of tumours with low baseline or post-trogocytic target expression, provided that CAR costimulatory features are adapted to target antigen density (FIG. 4E).

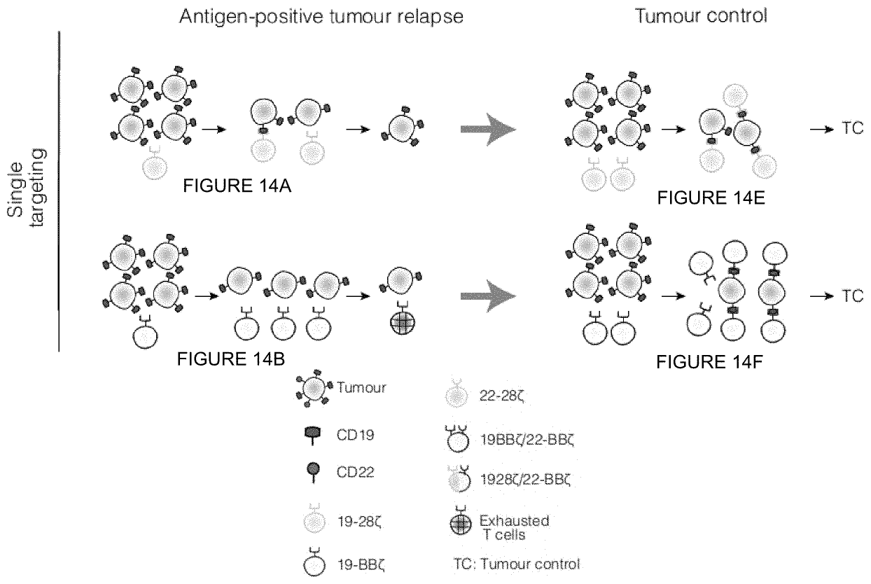

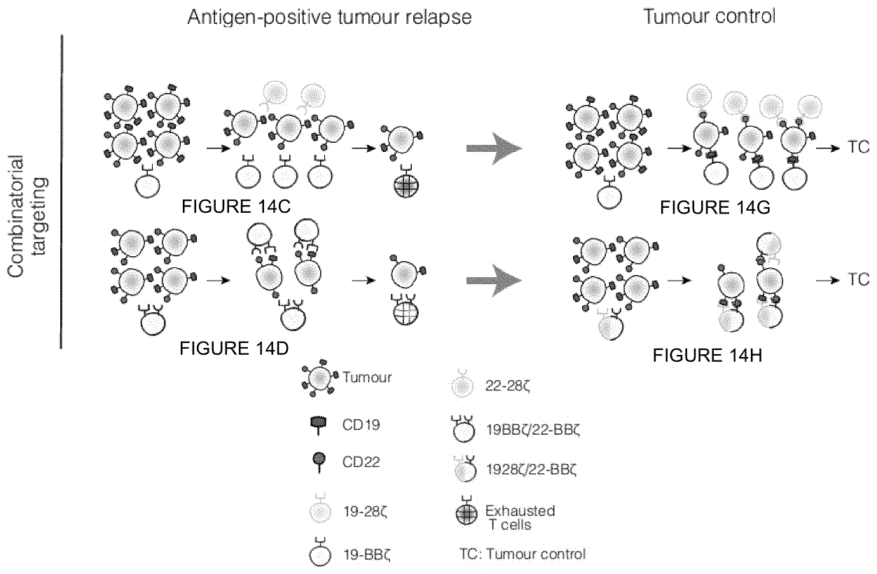

Different antigen expression profiles—original, negative or diminished—are associated with escape from immunotherapy^{4,6,7,9,14}. This study shows that tumour cells that are engaged but not killed by T cells are susceptible to trogocytic reduction of antigen density, with several possible consequences depending on antigen density, effector:target ratio and CAR design (FIG. 14). Understanding these dynamic features provides a framework for rational CAR T cell dosing and combinatorial targeting strategies, including target-adapted CAR costimulatory functions.

### REFERENCES

Sadelain, M., Riviere, I. & Riddell, S. Therapeutic T cell engineering. Nature 545, 423-431, (2017). 2 June, C. H. & Sadelain, M. Chimeric Antigen Receptor Therapy. The New England journal of medicine 379, 64-73, (2018).
3 Brudno, J. N. & Kochenderfer, J. N. Chimeric antigen receptor T-cell therapies for lymphoma. Nature reviews. Clinical oncology 15, 31-46, (2018).
4 Brudno, J. N. et al. T Cells Genetically Modified to Express an Anti-B-Cell Maturation Antigen Chimeric Antigen Receptor Cause Remissions of Poor-Prognosis Relapsed Multiple Myeloma. Journal of clinical oncology 36, 2267-2280, (2018).
5 Majzner, R. G. & Mackall, C. L. Tumor Antigen Escape from CAR T-cell Therapy. Cancer discovery, (2018).
6 Sotillo, E. et al. Convergence of Acquired Mutations and Alternative Splicing of CD19 Enables Resistance to CART-19 Immunotherapy. Cancer discovery 5, 1282-1295, (2015).
7 Fry, T. J. et al. CD22-targeted CART cells induce remission in B-ALL that is naive or resistant to CD19-targeted CAR immunotherapy. Nature medicine 24, 20-28, (2018).
8 Gardner, R. et al. Acquisition of a CD19-negative myeloid phenotype allows immune escape of MLL-rearranged B-ALL from CD19 CAR-T-cell therapy. Blood 127, 2406-2410, (2016).
9 Orlando, E. J. et al. Genetic mechanisms of target antigen loss in CAR19 therapy of acute lymphoblastic leukemia. Nature medicine 24, 1504-1506, (2018).
10 Brentj ens, R. J. et al. Eradication of systemic B-cell tumors by genetically targeted human T lymphocytes co-stimulated by CD80 and interleukin-15. Nature medicine 9, (2003).
11 Barrett, D. M. et al. Noninvasive bioluminescent imaging of primary patient acute lymphoblastic leukemia: a strategy for preclinical modeling. Blood 118, el12-117, (2011).
12 Zhao, Z. et al. Structural Design of Engineered Costimulation Determines Tumor Rejection Kinetics and Persistence of CART Cells. Cancer cell 28, 415-428, (2015).
13 Lee, D. W. et al. T cells expressing CD19 chimeric antigen receptors for acute lymphoblastic leukaemia in children and young adults: a phase 1 dose-escalation trial. Lancet 385, 517-528, (2015).
14 Maude, S. L. et al. Tisagenlecleucel in Children and Young Adults with B-Cell Lymphoblastic Leukemia. The New England journal of medicine 378, 439-448, (2018).
15 Turtle, C. J. et al. CD19 CAR-T cells of defined CD4+:CD8+composition in adult B cell ALL patients. The Journal of clinical investigation 126, 2123-2138, (2016).
16 Park, J. H. et al. Long-Term Follow-up of CD19 CAR Therapy in Acute Lymphoblastic Leukemia. The New England journal of medicine 378, 449-459, (2018).
17 Martinez-Martin, N. et al. T cell receptor internalization from the immunological synapse is mediated by TC21 and RhoG GTPase-dependent phagocytosis. Immunity 35, 208-222, (2011).
18 Priceman, S. J. et al. Co-stimulatory signaling determines tumor antigen sensitivity and persistence of CAR T cells targeting PSCA+metastatic prostate cancer. Oncoimmunology 7, e1380764, (2018).
19 Halle, S. et al. In Vivo Killing Capacity of Cytotoxic T Cells Is Limited and Involves Dynamic Interactions and T Cell Cooperativity. Immunity 44, 233-245, (2016).
20. Xiao X, Ho M, Zhu Z, Pastan I, Dimitrov DS. Identification and characterization of fully human anti-CD22 monoclonal antibodies. MAbs.1(3):297-303(2009)
21. Haso, W. et al. Anti-CD22-chimeric antigen receptors targeting B-cell precursor acute lymphoblastic leukemia. Blood. 121(7):1165-74 (2013).

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. An immunoresponsive cell comprising
a) a first CAR comprising a first extracellular antigen-binding domain that binds to a first antigen and a first intracellular signaling domain comprising a first co-stimulatory molecule or a portion thereof, wherein the first co-stimulatory molecule is CD28; and
b) a second CAR comprising a second extracellular antigen-binding domain that binds to a second antigen and a second intracellular signaling domain comprising a second co-stimulatory molecule or a portion thereof, wherein the second co-stimulatory molecule is different from the first co-stimulatory molecule.

2. The immunoresponsive cell of claim 1, wherein the first antigen has a density level of less than about 5,000 molecules per cell, between about 5,000 molecules per cell and about 10,000 per cell molecules per cell, or more than about 10,000 molecules per cell on the surface of the target cell.

3. The immunoresponsive cell of claim 1, wherein the first intracellular signaling domain comprises a modified or a native CD3ζ polypeptide.

4. The immunoresponsive cell of claim 3, wherein the modified CD3ζ polypeptide comprises one or more ITAM variant comprising one or more loss-of-function mutations, wherein each of the one or more ITAM variant is independently selected from the group consisting of an ITAM1 variant, an ITAM2 variant, and an ITAM3 variant.

5. The immunoresponsive cell of claim 4, wherein the modified CD3ζ polypeptide comprises or consists essentially of or consists of a native ITAM1, an ITAM2 variant, and an ITAM3 variant.

6. The immunoresponsive cell of claim 5, wherein the native ITAM1 consists of the amino acid sequence set forth in SEQ ID NO: 23, the ITAM2 variant consists of the amino acid sequence set forth in SEQ ID NO: 29, and the ITAM3 variant consists of the amino acid sequence set forth in SEQ ID NO: 33, optionally wherein the modified CD3ζ polypeptide consists of the amino acid sequence set forth in SEQ ID NO: 135.

7. The immunoresponsive cell of claim 5, wherein the first antigen has a density of between about 5,000 molecules per cell and about 10,000 per cell molecules per cell, or more than about 10,000 molecules per cell on the surface of the target cell.

8. The immunoresponsive cell of claim 1, wherein the first intracellular signaling domain comprises a native CD3ζ polypeptide.

9. The immunoresponsive cell of claim 8, wherein the first antigen has a density of less than about 5,000 molecules per cell on the surface of the target cell.

10. The immunoresponsive cell of claim 1, wherein the second co-stimulatory molecule is selected from the group consisting of 4-1BB, OX40, DAP-10, CD27, CD4O/My88, NKGD2, and combinations thereof.

11. The immunoresponsive cell of claim 1, wherein the second co-stimulatory molecule is 4-1BB.

12. The immunoresponsive cell of claim 1, wherein the second antigen has a density level of more than about 10,000 molecules per cell, between about 5,000 molecules per cell and about 10,000 per cell molecules per cell, or less than about 5,000 molecules per cell on the surface of the target cell.

13. The immunoresponsive cell of claim 1, wherein the second intracellular signaling domain comprises a native or a modified CD3ζ polypeptide.

14. The immunoresponsive cell of claim 13, wherein the modified CD3ζ polypeptide comprises one or more ITAM variant comprising one or more loss-of-function mutations, wherein each of the one or more ITAM variant is independently selected from the group consisting of an ITAM1 variant, an ITAM2 variant, and an ITAM3 variant.

15. The immunoresponsive cell of claim 14, wherein the modified CD3ζ polypeptide comprises or consists essentially of or consists of a native ITAM1, an ITAM2 variant, and an ITAM3 variant.

16. The immunoresponsive cell of claim 15, wherein the native ITAM1 consists of the amino acid sequence set forth in SEQ ID NO: 23, the ITAM2 variant consists of the amino acid sequence set forth in SEQ ID NO: 29, and the ITAM3 variant consists of the amino acid sequence set forth in SEQ ID NO: 33, optionally wherein the modified CD3ζ polypeptide consists of the amino acid sequence set forth in SEQ ID NO: 135.

17. The immunoresponsive cell of claim 15, wherein the second intracellular signaling domain comprises two copies of a modified or native CD3ζ polypeptide.

18. The immunoresponsive cell of claim 15, wherein the second antigen has a density level of more than about 10,000 molecules per cell on the surface of the target cell.

19. The immunoresponsive cell of claim 1, wherein the second intracellular signaling domain comprises a native CD3ζ polypeptide.

20. The immunoresponsive cell of claim 19, wherein the second antigen has a density level of between about 5,000 molecules per cell and about 10,000 per cell molecules per cell, or less than about 5,000 molecules per cell on the surface of the target cell.

21. The immunoresponsive cell of claim 1, further comprising a third CAR comprising a third extracellular antigen-binding domain that binds to a third antigen and a third intracellular signaling domain.

22. The immunoresponsive cell of claim 21, wherein third intracellular signaling domain does not comprise a co-stimulatory molecule.

23. The immunoresponsive cell of claim 21, wherein third intracellular signaling domain comprises a third co-stimulatory molecule or a portion thereof

24. The immunoresponsive cell of claim 23, wherein the third co-stimulatory molecule is selected from the group consisting of 4-1BB, ICOS, OX40, DAP-10, CD27, CD4O/My88, NKGD2, and combinations thereof.

25. The immunoresponsive cell of claim 21, wherein the third antigen has a density level of more than about 10,000 molecules per cell, or between about 5,000 molecules per cell and about 10,000 molecules per cell, or less than about 5,000 molecules per cell on the surface of the target cell, and wherein the third co-stimulatory molecule is a CD28 polypeptide.

26. The immunoresponsive cell of claim 1, wherein each of the first and second antigens is independently selected from tumor antigens, pathogen antigens, and combinations thereof.

27. The immunoresponsive cell of claim 1, wherein each of the first and second antigens is a tumor antigen.

28. The immunoresponsive cell of claim 26, wherein the tumor antigen is selected from the group consisting of carbonic anhydrase IX (CAIX), carcinoembryonic antigen (CEA), CD8, CD7, CD10, CD19, CD20, CD22, CD30, CD33, CLL1, CD34, CD38, CD41, CD44, CD49f, CD56, CD74, CD133, CD138, CD123, CD44V6, epithelial glycoprotein-2 (EGP-2), epithelial glycoprotein-40 (EGP-40), epithelial cell adhesion molecule (EpCAM), receptor tyrosine-protein kinases Erb-B2, Erb-B3, Erb-B4, folate-binding protein (FBP), fetal acetylcholine receptor (AChR), folate receptor-a, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Ra2), x-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), MAGEA3, p53, MART1, GP100, Proteinase3 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, CD99, CD70, ADGRE2, CCR1, LILRB2, PRAME, CCR4, CDS, CD3, TRBC1, TRBC2, TIM-3, Integrin B7, ICAM-1, and CLEC12A.

29. The immunoresponsive cell of claim 1, wherein the first antigen is CD19 and the second antigen is CD22.

30. A composition comprising an immunoresponsive cell of claim 1.

31. The composition of claim 30, which is a pharmaceutical composition that comprises a pharmaceutically acceptable excipient.

32. A method of reducing tumor burden in a subject, treating a subject having a relapse of a neoplasm, treating and/or preventing a pathogen infection in a subject, and/or treating and/or preventing an infectious disease in a subject, the method comprising administering to the subject an effective amount of the immunoresponsive cells of claim 1.

33. A nucleotide acid composition, comprising:
a) a first nucleotide sequence encoding a first CAR comprising a first extracellular antigen-binding domain that binds to a first antigen and a first intracellular signaling domain comprising a first co-stimulatory molecule or a portion thereof, wherein the first co-stimulatory molecule is CD28; and
b) a second nucleotide sequence encoding a second CAR comprising a second extracellular antigen-binding domain that binds to a second antigen and a second intracellular signaling domain comprising a second co-stimulatory molecule or a portion thereof, wherein the second co-stimulatory molecule is different from the first co-stimulatory molecule.

34. A method for producing an antigen-specific immunoresponsive cell, the method comprising introducing into an immunoresponsive cell a nucleic acid composition of claim 33.

35. A kit comprising an immunoresponsive cell of claim 1.


SEQUENCE LISTING (150 sequences)

SEQ ID NO: 1 - protein, 345 aa, Homo sapiens
MAQHHLWILLLCLQTWPEAAGKDSEIFTVNGILGESVTFPVNIQEPRQVKIIAWTSKTSVAYVTPGDSETAPVVTVTHRNYYERIHALGPNYNLVISDLRMEDAGDYKADINTQADPYTTTKRYNLQIYRRLGKPKITQSLMASVNSTCNVTLTCSVEKEEKNVTYNWSPLGEEGNVLQIFQTPEDQELTYTCTAQNPVSNNSDSISARQLCADIAMGFRTHHTGLLSVLAMFFLLVLILSSVFLFRLFKRRQGRIFPEGSCLNTFTKNPYAASKKTIYTYIMASRNTQPAESRIYDEILQSKVLPSKEEPVNTVYSEVQFADKMGKASTQDSKPPGTSSYEIVI

SEQ ID NO: 2 - DNA, 75 bp, Homo sapiens
ttgctgagcgtgctggctatgttctttctgcttgttctcattctgtcttcagtgtttttgttccgtttgttcaag

SEQ ID NO: 3 - protein, 583 aa, Homo sapiens
MESKGASSCRLLFCLLISATVFRPGLGWYTVNSAYGDTIIIPCRLDVPQNLMFGKWKYEKPDGSPVFIAFRSSTKKSVQYDDVPEYKDRLNLSENYTLSISNARISDEKRFVCMLVTEDNVFEAPTIVKVFKQPSKPEIVSKALFLETEQLKKLGDCISEDSYPDGNITWYRNGKVLHPLEGAVVIIFKKEMDPVTQLYTMTSTLEYKTTKADIQMPFTCSVTYYGPSGQKTIHSEQAVFDIYYPTEQVTIQVLPPKNAIKEGDNITLKCLGNGNPPPEEFLFYLPGQPEGIRSSNTYTLTDVRRNATGDYKCSLIDKKSMIASTAITVHYLDLSLNPSGEVTRQIGDALPVSCTISASRNATVVWMKDNIRLRSSPSFSSLHYQDAGNYVCETALQEVEGLKKRESLTLIVEGKPQIKMTKKTDPSGLSKTIICHVEGFPKPAIQWTITGSGSVINQTEESPYINGRYYSKIIISPEENVTLTCTAENQLERTVNSLNVSAISIPEHDEADEISDENREKVNDQAKLIVGIVVGLLLAALVAGVVYWLYMKKSKTASKHVNKDLGNMEENKKLEENNHKTEA

SEQ ID NO: 4 - DNA, 78 bp, Homo sapiens
ctaattgtgggaatcgttgttggtctcctccttgctgcccttgttgctggtgtcgtctactggctgtacatgaagaag

SEQ ID NO: 5 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 6 - DNA, 75 bp, Homo sapiens
atctacatctgggcgcccttggccgggacttgtggggtccttctcctgtcactggttatcaccctttactgcaac

SEQ ID NO: 7 - protein, 221 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQLRLHPLEKCSRMDY

SEQ ID NO: 8 - DNA, 75 bp, Homo sapiens
atcacccttggcctgctggtggctggcgtcctggttctgctggtttccctgggagtggccatccacctgtgctgc

SEQ ID NO: 9 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 10 - DNA, 75 bp, Homo sapiens
ttctggttacccataggatgtgcagcctttgttgtagtctgcattttgggatgcatacttatttgttggcttaca

SEQ ID NO: 11 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 12 - DNA, 75 bp, Homo sapiens
ttcctcctctggatccttgcagcagttagttcggggttgtttttttatagctttctcctcacagctgtttctttg

SEQ ID NO: 13 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 14 - DNA, 81 bp, Homo sapiens
attgtcatcatcactgtggtagcagccgcagtcataatgggcactgcaggcctcagcacgtacctctataaccgccagcgg

SEQ ID NO: 15 - protein, 39 aa, Artificial Sequence
  Synthetic
IEVMYPPPYLDNEKSNGTIIHVKGKHLCPSPLFPGPSKP

SEQ ID NO: 16 - DNA, 117 bp, Artificial Sequence
  Synthetic
caagagctgacttacacgtgtacagcccagaaccctgtcagcaacaattctgactccatctctgcccggcagctctgtgcagacatcgcaatgggcttccgtactcaccacaccggg

SEQ ID NO: 17 - DNA, 116 bp, Artificial Sequence
  Synthetic
accaactggagagaacagtaaactccttgaatgtctctgctataagtattccagaacacgatgaggcagacgagataagtgatgaaaacagagaaaaggtgaatgaccaggcaaaa

SEQ ID NO: 18 - DNA, 138 bp, Artificial Sequence
  Synthetic
cccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 19 - DNA, 117 bp, Artificial Sequence
  Synthetic
ctgagtgtggttgatttccttcccaccactgcccagcccaccaagaagtccaccctcaagaagagagtgtgccggttacccaggccagagacccagaagggcccactttgtagcccc

SEQ ID NO: 20 - DNA, 117 bp, Artificial Sequence
  Synthetic
tctcatgccaactattacttctgcaacctatcaatttttgatcctcctccttttaaagtaactcttacaggaggatatttgcatatttatgaatcacaactttgttgccagctgaag

SEQ ID NO: 21 - DNA, 117 bp, Artificial Sequence
  Synthetic
gacacgggactctacatctgcaaggtggagctcatgtacccaccgccatactacctgggcataggcaacggaacccagatttatgtaattgatccagaaccgtgcccagattctgac

SEQ ID NO: 22 - DNA, 117 bp, Homo sapiens
ggggaatcagtgactgtcactcgagatcttgagggcacctacctctgtcgggccaggagcactcaaggggaggtcacccgcaaggtgaccgtgaatgtgctctccccccggtatgag

SEQ ID NO: 23 - protein, 22 aa, Artificial Sequence
  Synthetic
QNQLYNELNLGRREEYDVLDKR

SEQ ID NO: 24 - DNA, 66 bp, Artificial Sequence
  Synthetic
cagaaccagctctataacgagctcaatctaggacgaagagaggagtacgatgttttggacaagaga

SEQ ID NO: 25 - protein, 22 aa, Artificial Sequence
  Synthetic
QNQLFNELNLGRREEFDVLDKR

SEQ ID NO: 26 - DNA, 66 bp, Artificial Sequence
  Synthetic
cagaaccagctctttaacgagctcaatctaggacgaagagaggagttcgatgttttggacaagaga

SEQ ID NO: 27 - protein, 23 aa, Artificial Sequence
  Synthetic
QEGLYNELQKDKMAEAYSEIGMK

SEQ ID NO: 28 - DNA, 69 bp, Artificial Sequence
  Synthetic
caggaaggcctgtacaatgaactgcagaaagataagatggcggaggcctacagtgagattgggatgaaa

SEQ ID NO: 29 - protein, 23 aa, Artificial Sequence
  Synthetic
QEGLFNELQKDKMAEAFSEIGMK

SEQ ID NO: 30 - DNA, 69 bp, Artificial Sequence
  Synthetic
caggaaggcctgttcaatgaactgcagaaagataagatggcggaggccttcagtgagattgggatgaaa

SEQ ID NO: 31 - protein, 22 aa, Artificial Sequence
  Synthetic
HDGLYQGLSTATKDTYDALHMQ

SEQ ID NO: 32 - DNA, 66 bp, Artificial Sequence
  Synthetic
cacgatggcctttaccagggtctcagtacagccaccaaggacacctacgacgcccttcacatgcag

SEQ ID NO: 33 - protein, 22 aa, Artificial Sequence
  Synthetic
HDGLFQGLSTATKDTFDALHMQ

SEQ ID NO: 34 - DNA, 66 bp, Artificial Sequence
  Synthetic
cacgatggccttttccaggggctcagtacagccaccaaggacaccttcgacgcccttcacatgcag

SEQ ID NO: 35 - protein, 5 aa, Artificial Sequence
  Synthetic
KRRGR

SEQ ID NO: 36 - DNA, 15 bp, Artificial Sequence
  Synthetic
aagagacgtggccgg

SEQ ID NO: 37 - protein, 5 aa, Artificial Sequence
  Synthetic
KPRRK

SEQ ID NO: 38 - DNA, 15 bp, Artificial Sequence
  Synthetic
aagccgagaaggaag

SEQ ID NO: 39 - protein, 8 aa, Artificial Sequence
  Synthetic
KGERRRGK

SEQ ID NO: 40 - DNA, 24 bp, Artificial Sequence
  Synthetic
aaaggcgagcgccggaggggcaag

SEQ ID NO: 41 - protein, 485 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - DNA, 1458 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 43 - protein, 485 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 44 - DNA, 1458 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgctaa

SEQ ID NO: 45 - protein, 416 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGHDGLYQGLSTATKDTYDALHMQALPPR

SEQ ID NO: 46 - DNA, 1251 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccacgatggcctttaccaggggctcagtacagccaccaaggacacctacgacgcccttcacatgcaggccctgccccctcgctaa

SEQ ID NO: 47 - protein, 416 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDP

SEQ ID NO: 48 - DNA, 1251 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggacccttaa

SEQ ID NO: 49 - protein, 485 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLFNELNLGRREEFDVLDKRRGRDPEMGGKPRRKNPQEGLFNELQKDKMAEAFSEIGMKGERRRGKGHDGLYQGLSTATKDTYDALHMQALPPR

SEQ ID NO: 50 - DNA, 1458 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctttaacgagctcaatctaggacgaagagaggagttcgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 51 - protein, 485 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLFNELNLGRREEFDVLDKRRGRDPEMGGKPRRKNPQEGLYNELQKDKMAEAYSEIGMKGERRRGKGHDGLYQGLSTATKDTYDALHMQALPPR

SEQ ID NO: 52 - DNA, 1458 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctttaacgagctcaatctaggacgaagagaggagttcgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 53 - protein, 485 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLFNELNLGRREEFDVLDKRRGRDPEMGGKPRRKNPQEGLYNELQKDKMAEAYSEIGMKGERRRGKGHDGLFQGLSTATKDTFDALHMQALPPR

SEQ ID NO: 54 - DNA, 1458 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctttaacgagctcaatctaggacgaagagaggagttcgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggccttttccaggggctcagtacagccaccaaggacaccttcgacgcccttcacatgcaggccctgccccctcgctaa

SEQ ID NO: 55 - protein, 485 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLFQGLSTATKDTFDALHMQALPPR

SEQ ID NO: 56 - DNA, 1458 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggccttttccaggggctcagtacagccaccaaggacaccttcgacgcccttcacatgcaggccctgccccctcgctaa

SEQ ID NO: 57 - protein, 455 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRR

SEQ ID NO: 58 - DNA, 1368 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggtaa

SEQ ID NO: 59 - DNA, 336 bp, Artificial Sequence
  Synthetic
agagtgaagtttagccggagtgccgatgctccagcctatcagcaaggacaaaaccaactttacaacgaacttaatctcggtaggcgagaggaatacgatgtgcttgataaacgccgaggtcgagatcccgaaatgggcgggaaaccgcgacgcaagaatcctcaagaaggactctataatgagttgcagaaggacaagatggctgaggcatatagtgagatcggtatgaagggagaacggagaagggggaaagggcatgatggattgtaccaaggacttagcacagctactaaagatacatatgacgccctgcacatgcaagcattgcctccacgc

SEQ ID NO: 60 - protein, 5 aa, Artificial Sequence
  Synthetic
GGGGS

SEQ ID NO: 61 - DNA, 15 bp, Artificial Sequence
  Synthetic
ggtgggggtggttcc

SEQ ID NO: 62 - protein, 10 aa, Artificial Sequence
  Synthetic
GGGGSGGGGS

SEQ ID NO: 63 - DNA, 30 bp, Artificial Sequence
  Synthetic
ggtgggggtggttccgggggaggcggctca

SEQ ID NO: 64 - DNA, 45 bp, Artificial Sequence
  Synthetic
ggtgggggtggttccgggggaggcggctcaggaggtggaggttct

SEQ ID NO: 65 - protein, 199 aa, Homo sapiens; identical to SEQ ID NO: 9

SEQ ID NO: 66 - protein, 15 aa, Artificial Sequence
  Synthetic
GGGGSGGGGSGGGGS

SEQ ID NO: 67 - protein, 20 aa, Homo sapiens
MYRMQLLSCIALSLALVTNS

SEQ ID NO: 68 - protein, 20 aa, Mus musculus
MYSMQLASCVTLTLVLLVNS

SEQ ID NO: 69 - protein, 20 aa, Homo sapiens
METPAQLLFLLLLWLPDTTG

SEQ ID NO: 70 - protein, 20 aa, Mus musculus
METDTLLLWVLLLWVPGSTG

SEQ ID NO: 71 - protein, 21 aa, Homo sapiens
MALPVTALLLPLALLLHAARP

SEQ ID NO: 72 - protein, 18 aa, Homo sapiens
MALPVTALLLPLALLLHA

SEQ ID NO: 73 - protein, 16 aa, Homo sapiens
MKWVTFISLLFSSAYS

SEQ ID NO: 74 - protein, 30 aa, Homo sapiens
MDSKGSSQKGSRLLLLLVVSNLLLCQGVVS

SEQ ID NO: 75 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 76 - protein, 6 aa, Artificial Sequence
  Synthetic
GYAFSS

SEQ ID NO: 77 - protein, 6 aa, Artificial Sequence
  Synthetic
YPGDGD

SEQ ID NO: 78 - protein, 9 aa, Artificial Sequence
  Synthetic
KTISSVVDF

SEQ ID NO: 79 - protein, 11 aa, Artificial Sequence
  Synthetic
KASQNVGTNVA

SEQ ID NO: 80 - protein, 6 aa, Artificial Sequence
  Synthetic
SATYRN

SEQ ID NO: 81 - protein, 9 aa, Artificial Sequence
  Synthetic
QQYNRYPYT

SEQ ID NO: 82 - protein, 122 aa, Artificial Sequence
  Synthetic
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 83 - protein, 108 aa, Artificial Sequence
  Synthetic
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 84 - protein, 263 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 85 - DNA, 789 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 86 - protein, 235 aa, Homo sapiens; identical to SEQ ID NO: 5

SEQ ID NO: 87 - protein, 247 aa, Mus musculus
MASPLTRFLSLNLLLMGESIILGSGEAKPQAPELRIFPKKMDAELGQKVDLVCEVLGSVSQGCSWLFQNSSSKLPQPTFVVYMASSHNKITWDEKLNSSKLFSAVRDTNNKYVLTLNKFSKENEGYYFCSVISNSVMYFSSVVPVLQKVNSTTTKPVLRTPSPVHPTGTSQPQRPEDCRPRGSVKGTGLDFACDIYIWAPLAGICVAPLLSLIITLICYHRSRKRVCKCPRPLVRQEGKPRPSEKIV

SEQ ID NO: 88 - protein, 69 aa, Artificial Sequence
  Synthetic
STTTKPVLRTPSPVHPTGTSQPQRPEDCRPRGSVKGTGLDFACDIYIWAPLAGICVALLLSLIITLICY

SEQ ID NO: 89 - DNA, 207 bp, Artificial Sequence
  Synthetic
tctactactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctac

SEQ ID NO: 90 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 91 - DNA, 81 bp, Artificial Sequence
  Synthetic
ttttgggtgctggtggtggttggtggagtcctggcttgctatagcttgctagtaacagtggcctttattattttctgggtg

SEQ ID NO: 92 - protein, 27 aa, Artificial Sequence
  Synthetic
FWVLVVVGGVLACYSLLVTVAFIIFWV

SEQ ID NO: 93 - DNA, 81 bp, Artificial Sequence; identical to SEQ ID NO: 91
  Synthetic

SEQ ID NO: 94 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 95 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - DNA, 336 bp, Artificial Sequence
  Synthetic
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 97 - protein, 218 aa, Artificial Sequence
  Synthetic
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 98 - DNA, 123 bp, Artificial Sequence
  Synthetic
aatagtagaaggaacagactccttcaaagtgactacatgaacatgactccccggaggcctgggctcactcgaaagccttaccagccctacgcccctgccagagactttgcagcgtaccgcccc

SEQ ID NO: 99 - protein, 41 aa, Artificial Sequence
  Synthetic
NSRRNRLLQSDYMNMTPRRPGLTRKPYQPYAPARDFAAYRP

SEQ ID NO: 100 - DNA, 123 bp, Artificial Sequence; identical to SEQ ID NO: 98
  Synthetic

SEQ ID NO: 101 - protein, 41 aa, Artificial Sequence
  Synthetic
RSKRSRLLHSDYMNMTPRRPGPTRKHYQPYAPPRDFAAYRS

SEQ ID NO: 102 - DNA, 123 bp, Artificial Sequence
  Synthetic
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 103 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 104 - protein, 42 aa, Artificial Sequence
  Synthetic
KRGRKKLLYIFKQPFMRPVQTTQEEDGCSCRFPEEEEGGCEL

SEQ ID NO: 105 - DNA, 126 bp, Artificial Sequence
  Synthetic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 106 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 107 - protein, 22 aa, Artificial Sequence
  Synthetic
GSGATNFSLLKQAGDVEENPGP

SEQ ID NO: 108 - protein, 41 aa, Artificial Sequence
  Synthetic
RSKRSRLLHSDYMNMTPRRPGPTRKHYQPYAPPRDFAAYRK

SEQ ID NO: 109 - protein, 39 aa, Artificial Sequence
  Synthetic
NQLERTVNSLNVPAISIPEHDEADEISDENREKVNDQAK

SEQ ID NO: 110 - protein, 42 aa, Artificial Sequence
  Synthetic
AAANQLERTVNSLNVSAISIPEHDEADEISDENREKVNDQAK

SEQ ID NO: 111 - protein, 48 aa, Artificial Sequence
  Synthetic
PEHDEADEISDENREKVNDQAKLIVGIVVGLLLAALVAGVVYWLYMKK

SEQ ID NO: 112 - protein, 37 aa, Artificial Sequence
  Synthetic
ENREKVNDQAKLIVGIVVGLLLAALVAGVVYWLYMKK

SEQ ID NO: 113 - protein, 65 aa, Artificial Sequence
  Synthetic
NQLERTVNSLNVPAISIPEHDEADEISDENREKVNDQAKLIVGIVVGLLLAALVAGVVYWLYMKK

SEQ ID NO: 114 - protein, 66 aa, Artificial Sequence
  Synthetic
TCTAENQLERTVNSLNVSAISIPEHDEADEISDENREKVNDQAKLIVGIVVGLLLAALVAGVVYWL

SEQ ID NO: 115 - protein, 44 aa, Artificial Sequence
  Synthetic
PEHDEADEISDENREKVNDQAKLIVGIVVGLLLAALVAGVVYWL

SEQ ID NO: 116 - protein, 61 aa, Artificial Sequence
  Synthetic
NQLERTVNSLNVSAISIPEHDEADEISDENREKVNDQAKLIVGIVVGLLLAALVAGVVYWL

SEQ ID NO: 117 - protein, 68 aa, Artificial Sequence
  Synthetic
AAANQLERTVNSLNVSAISIPEHDEADEISDENREKVNDQAKLIVGIVVGLLLAALVAGVVYWLYMKK

SEQ ID NO: 118 - protein, 491 aa, Artificial Sequence
  Synthetic
MALPVTALLLPLALLLHADIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKRGGGGSGGGGSGGGGSEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 119 - DNA, 1476 bp, Artificial Sequence
  Synthetic
atggctctcccagtgactgccctactgcttcccctagcgcttctcctgcatgcagacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacggggtggaggtggatcaggtggaggtggatctggtggaggtggatctgaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcagcggccgcacccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 120 - protein, 477 aa, Artificial Sequence
  Synthetic
MELGLSWIFLLAILKGVQCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - DNA, 1434 bp, Artificial Sequence
  Synthetic
atggaactcggtctgtcctggatttttctgcttgcgatcttgaaaggagtgcagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggaggaggaggcagcgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaagcggccgccattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 122 - protein, 483 aa, Artificial Sequence
  Synthetic
MELGLSWIFLLAILKGVQCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1452 bp, Artificial Sequence
  Synthetic
atggaactcggtctgtcctggatttttctgcttgcgatcttgaaaggagtgcagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggaggaggaggcagcgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaagcggccgcacccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgtaaccccctcgc

SEQ ID NO: 124 - protein, 7 aa, Artificial Sequence
  Synthetic
GVSLPDY

SEQ ID NO: 125 - protein, 5 aa, Artificial Sequence
  Synthetic
WGSET

SEQ ID NO: 126 - protein, 12 aa, Artificial Sequence
  Synthetic
HYYYGGSYAMDY

SEQ ID NO: 127 - protein, 11 aa, Artificial Sequence
  Synthetic
RASQDISKYLN

SEQ ID NO: 128 - protein, 7 aa, Artificial Sequence
  Synthetic
HTSRLHS

SEQ ID NO: 129 - protein, 9 aa, Artificial Sequence
  Synthetic
QQGNTLPYT

SEQ ID NO: 130 - protein, 124 aa, Artificial Sequence
  Synthetic
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 131 - protein, 107 aa, Artificial Sequence
  Synthetic
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 132 - protein, 255 aa, Artificial Sequence
  Synthetic
MELGLSWIFLLAILKGVQCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 133 - DNA, 765 bp, Artificial Sequence
  Synthetic
atggaactcggtctgtcctggatttttctgcttgcgatcttgaaaggagtgcagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggaggaggaggcagcgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaa

SEQ ID NO: 134 - protein, 847 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 135 - protein, 112 aa, Artificial Sequence
  Synthetic
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 136 - DNA, 336 bp, Artificial Sequence
  Synthetic
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgc

SEQ ID NO: 137 - protein, 43 aa, Artificial Sequence
  Synthetic
RVKFSRSADAPAYQQGQNQLYNELNLGRREEYDVLDKRRGRDP

SEQ ID NO: 138 - DNA, 129 bp, Artificial Sequence
  Synthetic
agagtgaagttcagcaggagcgcagacgcccccgcgtaccagcagggccagaaccagctctataacgagctcaatctaggacgaagagaggagtacgatgttttggacaagagacgtggccgggaccct

SEQ ID NO: 139 - protein, 41 aa, Artificial Sequence; identical to SEQ ID NO: 108
  Synthetic

SEQ ID NO: 140 - DNA, 123 bp, Artificial Sequence
  Synthetic
aggagtaagaggagcaggctcctgcacagtgactacatgaacatgactccccgccgccccgggcccacccgcaagcattaccagccctatgccccaccacgcgacttcgcagcctatcgcaaa

SEQ ID NO: 141 - protein, 65 aa, Artificial Sequence
  Synthetic
NQLERTVNSLNVSAISIPEHDEADEISDENREKVNDQAKLIVGIVVGLLLAALVAGVVYWLYMKK

SEQ ID NO: 142 - DNA, 195 bp, Artificial Sequence
  Synthetic
aaccaactggagagaacagtaaactccttgaatgtctctgctataagtattccagaacacgatgaggcagacgagataagtgatgaaaacagagaaaaggtgaatgaccaggcaaaactaattgtgggaatcgttgttggtctcctccttgctgcccttgttgctggtgtcgtctactggctgtacatgaagaag

SEQ ID NO: 143 - protein, 120 aa, Artificial Sequence
  Synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 144 - protein, 107 aa, Artificial Sequence
  Synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 145 - DNA, 360 bp, Artificial Sequence
  Synthetic
gaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 146 - DNA, 320 bp, Artificial Sequence
  Synthetic
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcac

SEQ ID NO: 147 - protein, 610 aa, Artificial Sequence
  Synthetic
MELGLSWIFLLAILKGVQCQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKAAAPTTTPAPRPPTPAPTIASQPLSLRPEACRPAAGGAVHTRGLDFACDIYIWAPLAGTCGVLLLSLVITLYCNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGGGGSGGGGSGGGGSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - DNA, 1830 bp, Artificial Sequence
  Synthetic
atggaactcggtctgtcctggatttttctgcttgcgatcttgaaaggagtgcagtgccaagtacagcttcaacagtcaggtccggggctggtaaagccttctcagacactcagcctgacttgtgctataagtggggatagtgtttcatccaactcagcggcgtggaactggatccggcaaagtccatctcggggcctggagtggttggggcggacctattataggtctaagtggtacaatgattacgccgtctcagtgaagtcacggatcacaatcaatcccgatacgagtaagaatcagttctcacttcagcttaacagtgtgacacctgaagatacggcagtatattattgcgcgagagaggttactggggacctcgaagatgccttcgatatctggggtcaaggcacaatggttacagtcagctccggaggaggaggcagcgacatacagatgacacaatctccgagtagcctttccgcatccgtaggtgatagggttaccataacttgccgcgcatctcaaacgatctggtcctatctgaactggtaccagcagagaccaggaaaagctcctaatctgcttatctacgccgcaagctcactgcagtctggggttccgagtagattttctgggcgaggcagcggaacggattttactctgaccataagctctctgcaagcagaagattttgccacgtactactgccagcaatcttacagcatcccacaaacatttggacaaggcacaaagttggagatcaaagcggccgcacccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtgggggtggttccgggggaggcggctcaggaggtggaggttctagagtgaagtttagccggagtgccgatgctccagcctatcagcaaggacaaaaccaactttacaacgaacttaatctcggtaggcgagaggaatacgatgtgcttgataaacgccgaggtcgagatcccgaaatgggcgggaaaccgcgacgcaagaatcctcaagaaggactctataatgagttgcagaaggacaagatggctgaggcatatagtgagatcggtatgaagggagaacggagaagggggaaagggcatgatggattgtaccaaggacttagcacagctactaaagatacatatgacgccctgcacatgcaagcattgcctccacgc

SEQ ID NO: 149 - DNA, 19 bp, Artificial Sequence
  Synthetic
gaggctcagagagggtaag

SEQ ID NO: 150 - DNA, 17 bp, Artificial Sequence
  Synthetic
gtgccccggagagtctg